(19) United States
(12) Patent Application Publication
(10) Pub. No.: US 2019/0209538 A1
(43) Pub. Date: Jul. 11, 2019

(54) Precision Controlled Load and Release Particles for Post-Operative Pain

(71) Applicant: LIQUIDIA TECHNOLOGIES, INC., Morrisville, NC (US)

(72) Inventors: John Robert Savage, Chapel Hill, NC (US); Jacob J. Sprague, Cary, NC (US); Ashley Galloway, Cary, NC (US); Geoffrey Hird, Chapel Hill, NC (US); Marquita Nicole Lilly, Garner, NC (US); Akihisa Nonoyama, Apex, NC (US); Edward Graham Randles, Durham, NC (US); Benjamin Maynor, Durham, NC (US)

(73) Assignee: LIQUIDIA TECHNOLOGIES, INC., Morrisville, NC (US)

(21) Appl. No.: 16/099,118

(22) PCT Filed: May 5, 2017
(86) PCT No.: PCT/US17/31397

§ 371 Date: Nov. 5, 2018

Related U.S. Application Data
(60) Provisional application No. 62/332,015, filed on May 5, 2016.
(60) Provisional application No. 62/440,088, filed on Dec. 29, 2016.
(60) Provisional application No. 62/443,318, filed on Jan. 6, 2017.
(60) Provisional application No. 62/463,206, filed on Feb. 24, 2017.
(60) Provisional application No. 62/472,885, filed on Mar. 17, 2017.

(51) Int. Cl. A61K 31/445; A61K 47/26; A61K 47/36; A61K 47/10; A61K 31/167; A61K 31/47; A61K 31/381; A61K 9/00; A61K 9/16

(52) U.S. Cl. CPC A61K 31/445; A61K 47/26; A61K 47/36; A61K 47/10; A61K 31/167; A61P 23/00; A61K 31/381; A61K 9/0019; A61K 9/0014; A61K 9/1647; A61K 9/1682; A61K 31/47

(57) ABSTRACT

A composition to induce analgesia includes a plurality of particles, each particle of the plurality having 40-60 wt % amino amide anesthetic or a pharmaceutically acceptable salt, hydrate, or solvate thereof and 60-40 wt % PLGA polymer including 48:52 to 52:48 molar ratio D,L lactide:glycolide and an inherent viscosity of about 0.16 to 0.24 dL/g at 0.1% w/v in chloroform at 25° C. Each particle includes a non-spherical shape less than 100 μιτι in a broadest dimension, and having a volume of about 13,500 cubic micrometers. The amino amide anesthetic is crystalline and includes 50-70% crystalline form I and 30-50% crystalline form II.

### CROSS REFERENCE TO RELATED APPLICATIONS

This application claims priority to and the benefit of U.S. Provisional Patent Application No. 62/332,015, filed May 5, 2016, U.S. Provisional Patent Application No. 62/440,088, filed Dec. 29, 2016, U.S. Provisional Patent Application No. 62/443,318, filed Jan. 6, 2017, U.S. Provisional Patent Application No. 62/463,206, filed Feb. 24, 2017, and U.S. Provisional Patent Application No. 62/472,885, filed Mar. 17, 2017, all of which are incorporated herein by reference in their entireties.

### TECHNICAL FIELD OF THE INVENTION

This invention relates to drug particles of amino amide anesthetics, drug particles of amino amide anesthetics suspended in vehicles, methods of making the drug particles and vehicles, and use of the drug particles and optional vehicles.

### BACKGROUND OF THE FIELD OF THE INVENTION

It is estimated that more than 100 million surgical procedures are performed in the European Union and United States each year. Effective post-surgical pain management is a clinical imperative for every patient undergoing surgery. Infiltration of an amino amide anesthetic such as bupivacaine hydrochloride into the surgical site at closure can provide temporary analgesia. However, the period of analgesia may only last approximately 6 hours. Due to the limited duration of these local anesthetics, patients may be likely to experience early breakthrough pain before they are able to take or tolerate oral analgesics. In this case, use of strong parenteral analgesics, such as opioids, in the immediate post-surgical period may be necessary. There is a desire to limit patient exposure to strong analgesics, such as opioids, while still maintaining patient pain management. An existing treatment for post-surgical pain is EXPAREL (Pacira Pharmaceuticals, Inc., San Diego Calif.), which is a bupivacaine liposome injectable suspension, however, EXPAREL® is limited to a bupivacaine concentration of 13.3 mg/ml, a maximum dose of 266 mg of bupivacaine in 20 ml volume per treatment. Although certain treatment options exist, there remains an unmet need to extend pain control through the use of local anesthetics thereby delaying, decreasing, and/or eliminating the reliance on strong analgesics, such as opioids, in the post-surgical setting.

### SUMMARY OF THE INVENTION

In some embodiments, the present invention provides a composition including a plurality of particles, each particle of the plurality comprising 40-60 wt % amino amide anesthetic or a pharmaceutically acceptable salt, hydrate, or solvate thereof and 60-40 wt % polymer. In some embodiments, the polymer includes PLGA polymer, for example, PLGA comprising 48:52 to 52:48 molar ratio D,L lactide:glycolide and an inherent viscosity of about 0.16 to 0.24 dL/g at 0.1% w/v in chloroform at 25° C. In certain embodiments, each particle comprises a non-spherical shape less than 100 μm in a broadest dimension, and having a volume of about 13,500 cubic micrometers. In further embodiments, the amino amide anesthetic is crystalline and comprises 50-70% crystalline form I and 30-50% crystalline form II. In some embodiments, the amino amide anesthetic is selected from the group consisting of dibucaine, lidocaine, mepivacaine, prilocaine, bupivacaine, levobupivacaine, ropivacaine, articaine, etidocaine, and pharmaceutically acceptable salts, hydrates, and solvates thereof. In some such embodiments, the amino amide anesthetic comprises bupivacaine free base or pharmaceutically acceptable salts, hydrates, and solvates thereof. In further embodiments, each particle comprises a surface area of about 3500 square micrometers.

The composition according to some embodiments further comprises an aqueous vehicle comprising a viscosity modifier, a surfactant, a buffer, and, a tonicity modifier. The vehicle may have a viscosity less than about 50 cps. In some embodiments, the viscosity modifier comprises hyaluronic acid or a pharmaceutically acceptable salt thereof. In some embodiments, the viscosity modifier comprises sodium hyaluronate having an inherent viscosity of 1.6 to 2.2 m³/kg. In some embodiments, the viscosity modifier comprises sodium hyaluronate comprising about 0.5 to about 1.0 wt % of the vehicle. In some embodiments, the surfactant comprises polysorbate 80 or polysorbate 20 comprising from about 0.001 to 1.0 wt % of the vehicle. In some embodiments, the vehicle further comprises a surfactant selected from docusate sodium or sodium deoxycholate and optionally a co-solvent comprising ethanol, benzyl alcohol or glycerin.

A method of inducing extended analgesia, according to some embodiments of the present invention, includes administering to a site in need a composition comprising a plurality of particles, each particle of the plurality comprising 40-60 wt % amino amide anesthetic or a pharmaceutically acceptable salt, hydrate, or solvate thereof and 60-40 wt % polymer. In some embodiments, the polymer is a PLGA polymer, for example, PLGA polymer comprising 48:52 to 52:48 molar ratio D,L lactide:glycolide and an inherent viscosity of about 0.16 to 0.24 dL/g at 0.1% w/v in chloroform at 25° C. In some embodiments, each particle comprises a non-spherical shape less than 100 μm in a broadest dimension. In some embodiments, the particles provide three or more days of analgesia to the site in need. In some embodiments, administering comprises infiltration, injection or topical administration. In some embodiments, each particle of the plurality has a volume of about 13,500 cubic micrometers and a surface area of about 3500 square micrometers. In some embodiments, the amino amide anesthetic is crystalline and comprises 50-70% crystalline form I and 30-50% crystalline form II. In some embodiments, the amino amide anesthetic comprises bupivacaine free base or pharmaceutically acceptable salts, hydrates, and solvates thereof.

The method, in further embodiments, includes, before administering, suspending the particles in a vehicle comprising a viscosity modifier, a surfactant, a buffer, and, a tonicity modifier. The vehicle may have a viscosity less than about 50 cps. In some embodiments, the method includes, before suspending the particle in the vehicle, formulating the vehicle with a viscosity less than about 50 cps. In some embodiments, the viscosity modifier comprises sodium hyaluronate having an inherent viscosity of 1.6 to 2.2 m³/kg and comprises about 0.5 to about 1.0 wt % of the vehicle, and wherein the surfactant comprises polysorbate 80, polysorbate 20, docusate sodium or sodium deoxycholate and the vehicle optionally comprises a co-solvent comprising ethanol, benzyl alcohol or glycerin comprising from about 0.001 to 1.0 wt % of the vehicle.

In yet further embodiments, the present invention provides a formulation for administration to induce analgesia including a plurality of particles suspended in a vehicle comprising about 0.1 to 0.3 wt % viscosity modifier, about 4.0 wt % tonicity modifier, about 0.1 wt % surfactant, about 0.6 wt % buffer, a pH of about 7.7 to 8.3, and viscosity of about 30 to 50 cps. In some embodiments, each particle of the plurality comprises 40-60 wt % amino amide anesthetic or a pharmaceutically acceptable salt, hydrate, or solvate thereof and 60-40 wt % PLGA polymer comprising 48:52 to 52:48 molar ratio D,L lactide:glycolide and an inherent viscosity of about 0.16 to 0.24 dL/g at 0.1% w/v in chloroform at 25° C. In some embodiments, each particle comprises a non-spherical shape less than 100 μm in a broadest dimension and having a volume of about 13,500 cubic micrometers. In some embodiments, each particle comprises a surface area of about 3500 square micrometers. In some embodiments, the amino amide anesthetic is crystalline and comprises 50-70% crystalline form I and 30-50% crystalline form II. The amino amide anesthetic, in some embodiments, comprises bupivacaine free base or a pharmaceutically acceptable salt, hydrate, or solvate thereof.

The present invention, in some embodiments, also provides a method of forming an anesthetic particle. The method, in some embodiments, includes depositing a solution comprising 40-60 wt % amino amide anesthetic and 60-40 wt % PLGA onto a polymer mold comprising cavities having a volume of about 13500 cubic micrometers, positioning the solution into the cavities of the mold; and drying the solution while in the mold cavities to form crystalline amino amide anesthetic PLGA anesthetic particles, wherein the crystalline amino amide anesthetic comprises between 50-70% crystalline form I and 30-50% crystalline form II.

### BRIEF DESCRIPTION OF FIGURES

FIGS. 1A, 1B, and 1C depict rendering of a particle of the invention, a hexagonal prism.

FIG. 1A depicts a three-dimensional rendering of a particle of the invention, a hexagonal prism. The height of the hexagonal prism is approximately 25 μm. The width of the hexagonal prism face, representing the distance between two vertices with an intervening vertex, is approximately 25 μm. FIGS. 1B and 1C depict two-dimensional drawings of the hexagonal prism face and a cross-sectional view respectively. The length of each side of the hexagonal face, a in FIG. 1B, is calculated to be approximately 14.43 μm. FIG. 1C depicts a cross-sectional view of a hexagonal prism.

FIG. 2 depicts the latency for a control, Exparel (bupivacaine liposome injectable suspension) (Pacira Pharmaceuticals, Inc., San Diego, Calif.), bupivacaine particles, and PLGA/bupivacaine particles in an animal study.

FIGS. 3A, 3B, and 3C depicts hind paw withdrawal latencies for PLGA/bupivacaine particles, bupivacaine particles, and Exparel (bupivacaine liposome injectable suspension). FIG. 3A depicts the Mean±SEM left hind paw withdrawal latencies in vehicle (N=12) and PLGA/Bupivacaine Particles-dosed animals (N=11) at 2, 4, 5.5, and 7 hours post-dosing. All animals received vehicle (1.2 mL/kg) or PLGA/Bupivacaine Particles (33.3 mg/mL, 40 mg/kg), by perineural administration (++/+++: p<0.01/0.001 unpaired t-test versus vehicle group). FIG. 3B depicts Mean±SEM left hind paw withdrawal latencies in vehicle (N=12) and Bupivacaine Particles-dosed animals (N=11) at 2, 4, 5.5, and 7 hours post-dosing. All animals received vehicle (1.2 mL/kg) or Bupivacaine Particles (33.3 mg/mL, 40 mg/kg), by perineural administration (++/+++: p<0.01/0.001 unpaired t-test versus vehicle group). FIG. 3C depicts Mean±SEM left hind paw withdrawal latencies in vehicle (N=12) and Exparel (bupivacaine liposome injectable suspension) dosed animals (N=12) at 2, 4, 5.5, and 7 hours post-treatment). All animals received vehicle (1.2 mL/kg) or Exparel (bupivacaine liposome injectable suspension) (13.3 mg/mL, 18.6 mg/kg), by perineural administration (***: p<0.001 Dunnett's post hoc test versus BL). In FIG. 3C at each time interval, as in FIGS. 3A and 3B, the vehicle control is the left bar and the Exparel (bupivacaine liposome injectable suspension) is the right bar.

FIG. 4 depicts the bupivacaine plasma concentration (ng/mL) for bupivacaine particles of the invention and Exparel (bupivacaine liposome injectable suspension) for a pharmacokinetic study.

FIG. 5 depicts the bupivacaine plasma concentration (ng/mL) for PLGA/bupivacaine particles of the invention and Marcaine (bupivacaine hydrochloride solution (0.75%)) for a pharmacokinetic study.

FIG. 6 depicts the bupivacaine plasma concentration (ng/mL) for bupivacaine particles of the invention for a toxicity study.

FIG. 7 depicts the bupivacaine plasma concentration (ng/kg) for PLGA/bupivacaine particles of the invention for a toxicity study.

FIGS. 8A, 8B, and 8C depict the mean bupivacaine plasma concentration (ng/kg) for PLGA/bupivacaine particles and bupivacaine particles of the invention for a PK study. FIG. 8A depicts the mean bupivacaine plasma concentration (ng/mL) for the 2 mg/kg dose. FIG. 8B depicts the mean bupivacaine plasma concentration (ng/mL) for the 4 mg/kg dose. FIG. 8C depicts the mean bupivacaine plasma concentration (ng/mL) for the 6 mg/kg dose.

FIG. 9 depicts a 60×35 mm rectangular area (long axis oriented vertically) used in a fanning technique for delivery of PLGA/bupivacaine particles and bupivacaine particles in a clinical trial.

FIGS. 10A and 10B show plasma concentrations versus time for Cohort 1 dosed with particles of the present invention.

FIGS. 11A and 11B show plasma concentrations versus time for Cohort 2 dosed with particles of the present invention.

FIGS. 12A and 12B show plasma concentrations versus time for Cohort 3 dosed with particles of the present invention at a dose of 300 mg.

FIG. 13A shows patients from Cohort 4 and Cohort 5 dosed at 450 mg for LIQ865A. FIG. 13B shows patients from Cohort 4 dosed at 450 mg with LIQ865B. FIG. 13C shows log-linear collection of subjects shown in FIGS. 13A and 13B.

FIG. 14 shows plasma concentrations versus time for Cohort 5 dosed with particles of the invention at a dose of 600 mg.

FIG. 15 presents a log-linear plot including data for all subjects dosed at 450 mg (in Cohorts 4 and 5) and the subject dosed at 600 mg.

FIG. 16 shows a qualitative pharmacodynamics summary for 150 mg, 225 mg, 300 mg, and 450 mg doses.

FIG. 17 shows mechanical and cold detection thresholds for the individual subjects in Cohorts 1-4.

FIG. 18 shows comparison of plasma concentration (Cₘₐₓ) per patient per particle formulation type A compared to formulation type B for Cohorts 1-5.

### ABBREVIATIONS

API active pharmaceutical ingredient

AUC area under the curve

AVE average

BL baseline

Bup bupivacaine

CDT cold detection threshold

Cₘₐₓ maximum concentration

CMC carboxymethyl cellulose

cps centipoise

DMPC 1,2-Dimyristoyl-sn-glycero-3-phosphorylcholine

DOPC 1,2-Dioleoyl-sn-glycero-3-phosphocholine

DPPG 1,2-Dipal mitoyl-sn-g lycero-3-phosphorylglycerol sodium salt

DSPE 1,2-Distearoyl-sn-glycero-3-phosphoethanolamine

EDTA Ethylenediaminetetraacetic acid

ft/min feet/minute

g gram(s)

GMPG 1,2-ditetradecanoyl-sn-glycero-3-phospho-(1′-rac-glycerol) (sodium salt)

HCl hydrochloric acid

HDPE high-density polyethylene

HEPES (4-(2-hydroxyethyl)-1-piperazineethanesulfonic acid)

HPT heat pain threshold

kGy kilogray

LDPE low-density polyethylene

LIQ865A (865A) PLGA/bupivacaine drug particles

LIQ865B (865B) Bupivacaine drug particles

MDT mechanical detection threshold

mN milli-Newtons

MTD maximum tolerated dose

NA not applicable

NMT not more than

NT not tested

PD pharmacodynamics

PES polyethersulfone

PET polyethylene terephthalate

PGA poly(glycolic acid)

PK pharmacokinetics

pKa acid dissociation constant

PLA poly(lactic acid)

PLGA poly(lactic-co-glycolic acid)

PN perineural

ppm parts per million

PRINT Particle Replication In Non-Wetting Templates

psi pounds per square inch

PTFE polytetrafluoroethylene

PVOH polyvinyl alcohol

QS quantum satis, quantum sufficit

RH relative humidity

s second(s)

SC subcutaneous

SEM standard error of the mean

SN saphenous nerve

SQ subcutaneous

STDEV standard deviation

t_{1/2} half-life

TBD to be determined

Tₘₐₓ time of maximal concentration

TK toxicokinetics

Tris tris(hydroxymethyl)aminomethane

WDT warmth detection threshold

wt % weight percent

w/w weight per weight

XRPD x-ray powder diffraction

### DETAILED DESCRIPTION OF EMBODIMENTS OF THE INVENTION

A composition has been developed to provide analgesia through use of a sustained release composition for direct delivery to a site of interest. Generally delivery is accomplished during or post-surgery such that a reduction in prescription drugs is achieved. Delivery can be either injection, infiltration, deposition via a suspension, aerosolization, foam, paste or the like. The composition comprises a plurality of drug particles containing an amino amide anesthetic or pharmaceutically acceptable salt, hydrate, or solvate thereof and optionally a biocompatible polymer. The biocompatible polymer may be degradable, biodegradable, bioerodible, resorbable, and/or dissolvable. The applicants reference biocompatible polymer throughout in connection with the PLGA/bupivacaine drug particles and it will be understood by one of ordinary skill in the art the polymer is degradable, biodegradable, bioerodible, resorbable, and/or dissolvable. The plurality of particles may be delivered as particles and/or as particles suspended in a vehicle. A syringe, cannula, trocar, or other device containing a lumen may be used to administer via injection or infiltration. An aerosolization pump with or without a propellant may be used to administer aerosolization particle compositions. For topical administration the composition may be applied using any number of methods, including but not limited to, painting, swabbing, dabbing or aerosolization of particles for direct deposition onto a desired tissue or location.

Amino Amide Anesthetics

Amino amide anesthetics include dibucaine, lidocaine, mepivacaine, prilocaine, bupivacaine, levobupivacaine, ropivacaine, articaine, etidocaine, and pharmaceutically acceptable salts, hydrates, and/or solvates thereof. Preferably the amino amide is bupivacaine, levobupivacaine, and/or ropivacaine. More preferably the amino amide is bupivacaine and/or levobupivacaine. Most preferably the amino amide is bupivacaine. Preferably the bupivacaine is bupivacaine free base. Preferably the levobupivacaine is levobupivacaine free base.

Bupivacaine is a racemic mixture of two stereoisomers. Both ropivacaine and levobupivacaine are available as optically pure materials (single enantiomers). The pKa for bupivacaine, ropivacaine, and levobupivacaine are similar (N8.1). However, the clearance rates differ with ropivacaine clearing faster than bupivacaine clearing faster than levobupivacaine (˜0.72 L/min >0.58 L/min>0.32 L/min) [Adams A P, Grounds R M, Cashman Jeremy N. Recent Advances and Intensive Care, Inc. NetLibrary, 2002].

Pharmaceutically acceptable salts, hydrates, and/or solvates may have properties that differ from the free base version of the amino amide anesthetic. Use of the free base version may offer advantages over pharmaceutically acceptable salts, hydrates and/or solvates. For example, bupivacaine free base is less soluble than bupivacaine HCl at room temperature in an aqueous system between pH 7.0 and pH 9.0 [Shah J C and Maniar J J. J. Contr. Rel., 23, 261-270 (1993)]. Use of a less soluble amino amide anesthetic, such as for example bupivacaine free base, may provide additional analgesic benefit in an aqueous system, such as in the body of a mammal, by dissolving at a slower rate and therefore remaining persistent at the site of application and providing extended analgesic effect.

Biocompatible Polymers

The composition of the particle may also contain a biocompatible polymer. The biocompatible polymer may be biodegradable, bioerodable, resorbable, and/or dissolvable. In embodiments, the polymer materials used to form the drug particles described herein are biodegradable. In embodiments, the polymer materials may be any combination of polylactic acid, glycolic acid, and co-polymers thereof that provides sustained-release of the amino amide anesthetic agent over time, reduces conglomeration of particles, enhances stability of the drug substance, combinations thereof and the like.

Suitable polymeric materials or compositions for use in the drug particles include those materials which are compatible, which is biocompatible, with the body of a mammal so as to cause no substantial interference with the functioning or physiology of the body. Such polymeric materials may be biodegradable, bioerodible or both biodegradable and bioerodible.

In particular embodiments, examples of useful polymeric materials include, without limitation, such materials derived from and/or including organic esters and organic ethers, which when degraded result in physiologically acceptable degradation products. Also, polymeric materials derived from and/or including, anhydrides, amides, orthoesters and the like, by themselves or in combination with other monomers, may also find use in the present disclosure. The polymeric materials may be addition or condensation polymers. The polymeric materials may be cross-linked or non-cross-linked. For some embodiments, besides carbon and hydrogen, the polymers may include at least one of oxygen and nitrogen. The oxygen may be present as oxy, e.g. hydroxy or ether, carbonyl, e.g. non-oxo-carbonyl, such as carboxylic acid ester, and the like. The nitrogen may be present as amide, cyano and amino.

In one embodiment, polymers of hydroxyaliphatic carboxylic acids (e.g. polyesters), either homopolymers or copolymers are useful in the particles. Polyesters can include polymers of D-lactic acid, L-lactic acid, racemic lactic acid, glycolic acid, polycaprolactone, co-polymers thereof, and combinations thereof.

Some characteristics of the polymers or polymeric materials for use in embodiments of the present disclosure may include biocompatibility, compatibility with the selected amino amide anesthetic; ease of use of the polymer in making the particle delivery systems described herein, and desired sustained release profile.

In one embodiment, the biodegradable polymer matrix used to manufacture the particles is a synthetic aliphatic polyester, for example, a polymer of lactic acid and/or glycolic acid, and includes poly-(D,L-lactide) (PLA), poly-(D-lactide), poly-(L-lactide), polyglycolic acid (PGA), and/or the copolymer poly-(D, L-lactide-co-glycolide) (PLGA).

PLGA is synthesized through random ring-opening co-polymerization of the cyclic dimers of glycolic acid and lactic acid. Successive monomeric units of glycolic or lactic acid are linked together by ester linkages. The ratio of lactide to glycolide can be varied, altering the biodegradation characteristics of the product. By altering the ratio it is possible to tailor the polymer degradation time. Additional characteristics of the biocompatible polymer including, but not limited to, molecular weight, inherent viscosity, and crystallinity may also be modulated. Importantly, drug release characteristics are affected by the rate of biodegradation, molecular weight, and degree of crystallinity in drug delivery systems. By altering and customizing the biodegradable polymer, the drug delivery profile of the drug particles can be changed. PLA, PGA, and PLGA are cleaved predominantly by non-enzymatic hydrolysis of its ester linkages throughout the polymer matrix, in the presence of water in the surrounding tissues. PLA, PGA, and PLGA polymers are biocompatible, because they undergo backbone hydrolysis in the body to produce the original monomers, lactic acid and/or glycolic acid which are considered natural metabolites. Lactic and glycolic acids are nontoxic and eliminated safely via the Krebs cycle by conversion to carbon dioxide and water. The biocompatibility of PLA, PGA and PLGA polymers has been examined in tissues of animals and humans. The findings indicate that the polymers are well tolerated.

Examples of PLA polymers, which may be utilized in an embodiment of the disclosure, include but are not limited to, the RESOMER® Product line available from Evonik Industries identified as, but are not limited to, R 207 S, R 202 S, R 202 H, R 203 S, R 203 H, R 205 S, R 208, R 206, and R 104. Examples of suitable PLA polymers include both acid and ester terminated polymers with inherent viscosities ranging from approximately 0.15 to approximately 2.2 dL/g when measured at 0.1% w/v in CHCl₃ at 25° C. with an Ubbelhode size 0C glass capillary viscometer.

Examples of PLGA polymers, which may be utilized in an embodiment of the disclosure, include but are not limited to, the RESOMER® Product line from Evonik Industries identified as, but are not limited to, RG 502, RG 502 H, RG 503, RG 503 H, RG 504, RG 504 H, RG 505, RG 506, RG 653 H, RG 752 H, RG 752 S, RG 753 H, RG 753 S, RG 755, RG 755 S, RG 756, RG 756 S, RG 757 S, RG 750 S, RG 858, and RG 858 S. Such PLGA polymers include both acid and ester terminated polymers with inherent viscosities ranging from approximately 0.14 to approximately 1.7 dl/g when measured at 0.1% w/v in CHCl₃ at 25° C. with an Ubbelhode size 0C glass capillary viscometer. Example polymers used in various embodiments of the disclosure may include variation in the mole ratio of D,L-lactide to glycolide from approximately 50:50 to approximately 85:15, including, but not limited to, 50:50, 65:35, 75:25, and 85:15 as well as intervening ratios, for example 55:45 and the like.

The synthesis of various molecular weights of PLGA with various D,L-lactide-glycolide ratios is possible. In one embodiment, PLGA, such as RESOMER® RG502H, having a molar ratio of approximately 48:52 to 52:48 (D,L-lactide:glycolide) and an inherent viscosity of approximately 0.16 to approximately 0.24 dl/g when measured at 0.1% w/v in CHCl₃ at 25° C. with an Ubbelhode size 0C glass capillary viscometer may be used.

A few of the primary polymer characteristics that control amino amide anesthetic agent release rates are the molecular weight distribution, polymer endgroup (i.e., acid or ester), and the ratio of polymers and/or copolymers in the drug particle composition. The present disclosure provides examples of drug particle composition that possess desirable therapeutic agent, for example but not limited to amino amide anesthetics, release characteristics by manipulating one or more of the aforementioned properties.

The biodegradable polymeric materials which are included to form the drug particle's composition are often subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the active agent from the drug particles is the relative average molecular weight of the polymeric composition employed in the drug particles. Different molecular weights of the same or different polymeric compositions may be included to modulate the release profile of the at least one active agent, such as for example an amino amide anesthetic.

Particle Composition

The amino amide anesthetic may be formulated into the particles at a variety of concentrations. The amino amide anesthetic may comprise between 5 to 100 wt % of the particle. In alternative embodiments of the present invention, the amino amide anesthetic comprises between 20 and 99 wt % of the particle. In further alternative embodiments, the amino amide anesthetic comprises between 30 to 60 wt % of the particle. In certain embodiments, the wt % of the anesthetic component of the particle is chosen to comprise between 10, 20, 30, 40, 50, 60, 70, 80, 90, 95, 99, and 100 wt % of the particle. In an embodiment, the amino amide anesthetic comprises bupivacaine. In another embodiment, the amino amide anesthetic comprises levobupivacaine. In a particular embodiment, the amino amide anesthetic comprises ropivacaine. In an embodiment, the amino amide anesthetic comprises bupivacaine comprising between 90, 95, 99, and 100 wt % of the particle. In an embodiment, the amino amide anesthetic comprises bupivacaine comprising between 40, 50, and 60 wt % of the particle. In an embodiment, the amino amide anesthetic comprises bupivacaine comprising between 40 and 60 wt % of the particle. In an embodiment, the amino amide anesthetic comprises bupivacaine comprising between 50 and 60 wt % of the particle. In an embodiment, the amino amide anesthetic comprises levobupivacaine comprising between 90, 95, 99, and 100 wt % of the particle. Preferably the bupivacaine and/or levobupivacaine are the free base.

According to embodiments of the present invention where the particle includes more components than the amino amide anesthetic alone, the balance of the particle composition comprises a biocompatible polymer. The biocompatible polymer may comprise the balance of the particle, and therefore, according to the present invention may range between 99 to 1 wt % of the particle depending on the wt % anesthetic charge in the particle stock solution (described herein). In some embodiments the biocompatible polymer can comprise between 99, 95, 90, 80, 70, 60, 50, 40, 30, 20, 10, 5, 4, 3, 2, and 1 wt % of the particle. In preferred embodiments, the biocompatible polymer is PLA and/or PLGA. In a particular embodiment, the PLGA, comprises a molar ratio of approximately 48:52 to 52:48 (D,L-lactide:glycolide) and an inherent viscosity of approximately 0.16 to approximately 0.24 dl/g when measured at 0.1% w/v in CHCl₃ at 25° C. with an Ubbelhode size 0C glass capillary viscometer. In one embodiment, the PLGA comprises Resomer RG502H or Resomer RG502. In one embodiment, the PLGA comprises Resomer RG502H.

In one embodiment, the amino amide anesthetic comprises bupivacaine free base and the PLGA polymer comprises a molar ratio of approximately 48:52 to 52:48 (D,L-lactide:glycolide) and an inherent viscosity of approximately 0.16 to approximately 0.24 dl/g when measured at 0.1% w/v in CHCl₃ at 25° C. with an Ubbelhode size 0C glass capillary viscometer. In one embodiment, the amino amide anesthetic comprises bupivacaine free base and the PLGA polymer comprises Resomer RG502H. In one embodiment, the amino amide anesthetic comprises bupivacaine free base at about 50 to 60 wt % and the PLGA polymer comprises a molar ratio of approximately 48:52 to 52:48 (D,L-lactide:glycolide) and an inherent viscosity of approximately 0.16 to approximately 0.24 dl/g (when measured at 0.1% w/v in CHCl₃ at 25° C. with an Ubbelhode size 0C glass capillary viscometer) and is present in the particle at about 40 to 50 wt %. In a particular embodiment, particle are fabricated from a particle stock solution comprising, in acetone, bupivacaine free base at about 60 wt % and the PLGA polymer comprises a molar ratio of approximately 48:52 to 52:48 (D,L-lactide:glycolide) and an inherent viscosity of approximately 0.16 to approximately 0.24 dl/g (when measured at 0.1% w/v in CHCl₃ at 25° C. with an Ubbelhode size 0C glass capillary viscometer) at about 40 wt %. The particle are fabricated according to methods and materials contained herein.

In some embodiments, the drug particles of the present invention includes bupivacaine at about 40%-60 wt %, 42%-58 wt %, 44%-56 wt %, 46%-54 wt %, or 48%-52 wt %. In some embodiments, the drug particles of the present invention includes poly(lactic-co-glycolic) acid (PLGA) at about 40%-60 wt %, 42%-58 wt %, 44%-56 wt %, 46%-54 wt %, or 48%-52 wt %.

In some embodiments the active agent is in an amorphous state intermixed with the polymer matrix of the particle. In alternative embodiments, the active agent is in a crystallized form mixed with the polymer matrix of the particle. In some embodiments, the polymer matrix material degrades and the degradation assists release of the active agent. In some embodiments, the particle is about 100 percent active agent in crystalline form.

In some embodiments, the active agent (amino amide anesthetic) comprises more than one crystalline form or polymorph. For example, studies have identified two forms of crystalline bupivacaine free base: Form I, reported to be the thermodynamically stable form and Form II, a metastable form. It is thought that these two forms are monotropically related. Conversion from metastable Form II to thermodynamically stable Form I is not reversible. The crystalline bupivacaine free base may be present in Form I, Form II, and or Form I and Form II in a drug particle. The crystalline bupivacaine free base may be in one form, such as Form I, prior to dissolution in a solvent to form a particle stock solution. The crystalline bupivacaine free base may comprise the same form once drug particles are manufactured from the particle stock solution. For example, crystalline bupivacaine free base may be Form I prior to dissolution in a solvent to form a particle stock solution. The crystalline bupivacaine free base may comprise a different form once drug particles are manufactured from the particle stock solution and the active (bupivacaine free base) recrystallizes. For example, crystalline bupivacaine free base may comprise Form I prior to dissolution in a particle stock solution and comprise Form II once drug particles are manufactured. The active agent may comprise more than one form once drug particles are manufactured from the particle stock solution and the active recrystallizes. For example, crystalline bupivacaine free base may comprise Form I prior to dissolution in a particle stock solution and comprise Form I and Form II once drug particles are manufactured.

The form and/or form ratio of the active agent in the drug particles may change over time. The form and/or form ratio of the active agent in the drug particles may change over time at different rates under different storage conditions. For example, bupivacaine free base may comprise less than 40% Form I after being manufactured into drug particles. In some embodiments, the drug particles contain about 50% to about 70% Form I. In some embodiments, the drug particles contain about 55% to about 70% Form I. In some embodiments, the drug particles contain about 64±5% Form I. During storage, the bupivacaine free base may convert from metastable Form II to thermodynamically stable Form I. If stored at different conditions, such as −20° C., 2-8° C., or 25° C./60% relative humidity, the conversion of bupivacaine free base from metastable Form II to thermodynamically stable Form I may be accelerated at higher temperatures. In some embodiments, drug particles fabricated according to methods and materials disclosed and incorporated herein that include PLGA in the drug particle composition may exhibit a consistent crystalline form ratio over time.

Vehicle

Particles may be delivered as manufactured, i.e. dry, or may be delivered following suspension in a vehicle. Desired characteristics for a vehicle include, but are not limited to, biocompatibility, ability to disperse the particles, in use suspension stability, ability to be expressed through a device containing a lumen (e.g. syringe), maintain pH in the physiologic range, and maintain osmolarity. A suitable viscosity range for the vehicle is about 20 to 2,000 cps. In some embodiments, the viscosity is about 30 to 1,000 cps. In some embodiments, the viscosity is about 30 to 500 cps. In some embodiments, the viscosity is about 250 to 450 cps. In some embodiments, the viscosity is about 325 to 375 cps. In some embodiments, the viscosity is about 20 cps to 200 cps. In some embodiments, the viscosity is about 20 cps to 100 cps. In some embodiments, the viscosity is about 20 cps to 50 cps. In some embodiments, the viscosity is about 30 cps to 50 cps. In some embodiments, the viscosity is about 40 cps.

Vehicles used to deliver materials to patients most often are aqueous. Typical vehicles may contain one or more physiologically acceptable components in a buffer such as a saline, phosphate, Tris, borate, succinate, histidine, citrate or maleate buffer. A viscosity modifier may be added to improve in use suspension stability. Various buffering agents may be added to maintain pH in a physiologically acceptable range. Tonicity modifiers may be added to maintain osmolarity in a physiologically acceptable range. Surfactants or wetting agents may be added to reduce surface tension between the particles and vehicle to ease and improve dispersion of the particles into the vehicle.

Examples of viscosity modifiers include various polymeric materials including, but not limited to, poloxamers, carboxymethyl cellulose (CMC), hyaluronic acid-based polymers, and hyaluronate salts. A viscosity modifier may be added to increase the viscosity of the vehicle. The viscosity may be altered by incorporating more or less of a viscosity modifier of a given molecular weight. The viscosity may be altered by incorporating a given weight percent and incorporating a given viscosity modifier with a higher or lower molecular weight. More than one viscosity modifier may be used in some embodiments. In embodiments, the viscosity modifier comprises from about 0.1 to 5.0 wt % of the vehicle. In embodiments, the viscosity modifier comprises from about 0.25 to about 2.5 wt % of the vehicle. In embodiments, the viscosity modifier comprises from about 0.5 to about 1.25 wt % of the vehicle. In embodiments, the viscosity modifier comprises from about 0.1 to 0.5 wt % of the vehicle. In embodiments, the viscosity modifier comprises from about 0.1 to 0.3 wt % of the vehicle. In embodiments, the viscosity modifier comprises about 0.25 wt % of the vehicle. In embodiments, the viscosity modifier comprises sodium hyaluronate. In embodiments, the viscosity modifier comprises sodium hyaluronate having an inherent viscosity of about 1.6-2.2 m³/kg. In embodiments, the viscosity modifier comprises about 0.5 to 1.25 wt % sodium hyaluronate having an inherent viscosity of about 1.6-2.2 m³/kg. In embodiments, the viscosity modifier comprises about 0.1 to 0.3 wt % sodium hyaluronate having an inherent viscosity of about 1.6-2.2 m³/kg.

Examples of buffers include, but are not limited to, saline, phosphate, Tris, borate, succinate, histidine, citrate, acetate, tartrate, glutamate, glycine, bicarbonate, sulfate, nitrate, HEPES, or maleate buffers. Buffers are incorporated in the vehicle to maintain physiologically acceptable pH. The buffer should also maintain physiologically acceptable pH after the addition of any other materials, particularly when the particles are dispersed and/or suspended in the vehicle. Tris has a pKa of approximately 8 at 25° C., so Tris buffer has an effective pH range between 7.5 and 9.0. Tris is available in both acid, Tris HCl, and base, Tris base. In embodiments, the buffer comprises Tris base. In embodiments, the buffer comprises Tris HCl. In embodiments, the buffer comprises Tris base and Tris HCl. In embodiments, the buffer comprises Tris base and the pH adjustment is made using HCl. In embodiments, the buffer comprises Tris base and the pH adjustment is made using Tris HCl. In embodiments, the buffer comprises Tris HCl and the pH adjustment is made using NaOH. In embodiments, the buffer comprises Tris HCl and the pH adjustment is made using Tris base. Approximately 0.4 to 0.8 wt % of Tris may be used to prepare the vehicle in certain embodiments of the present invention. In alternative embodiments of the present invention, approximately 0.5 to 0.7 wt % Tris may be used to prepare the vehicle. In further embodiments, approximately 0.61 wt % of Tris may be used to prepare the vehicle. In embodiments, the pH of the vehicle may be between about pH 6.0 to and 9.0. In embodiments, the pH of the vehicle may be between about pH 7.0 and 8.5. In embodiments, the pH of the vehicle may be between about pH 7.5 and 8.5. In embodiments, the pH of the vehicle may be between about 7.7 and 8.3. In embodiments, the pH of the vehicle may be about 8.0. In embodiments, the buffer comprises Tris base and Tris HCl and the pH is between about 7.7 and 8.3.

Surfactants or wetting agents may be added to reduce surface tension between the drug particles and vehicle to ease and/or improve dispersion of the particles into the vehicle. Surfactants may be cationic, anionic, zwitterionic, or non-ionic. Exemplary anionic surfactants include docusate (dioctyl sodium sulfosuccinate). Exemplary non-ionic surfactants and wetting agents include polysorbates (polyoxyethylene glycol sorbitan alkyl esters), sodium deoxycholate, and poloxamers (block copolymers of polyethylene glycol and polypropylene glycol). Examples of polysorbates include polysorbate 20 and polysorbate 80. In embodiments, the surfactant or wetting agent may comprise from about 0.001 to 1.0 wt % of the vehicle. In embodiments, the surfactant or wetting agent may comprise from about 0.01 to 1.0 wt % of the vehicle. In embodiments, the surfactant or wetting agent may comprise from about 0.05 to 0.5 wt % of the vehicle. In embodiments, the surfactant or wetting agent may comprise from about 0.05 to 0.25 wt % of the vehicle. In embodiments, the surfactant or wetting agent may comprise from about 0.05 to 0.15 wt % of the vehicle. In embodiments, the surfactant or wetting agent may comprise about 0.1 wt % of the vehicle. In embodiments, the surfactant or wetting agent comprises polysorbate 80. In embodiments, the surfactant or wetting agent comprises about 0.05 to 0.15 wt % polysorbate 80.

Various substances may be added to modify the osmolarity of the vehicle. In embodiments, the osmolarity is between about 200 and 400 mOs/kg. In embodiments, the osmolarity is between about 250 and 350 mOs/kg. Tonicity modifiers may be added to the vehicle to adjust the osmolarity of the vehicle. Depending on the tonicity modifier chosen, from about 0.2 to 5.0 wt % may be used. Tonicity modifiers should be biocompatible. Tonicity modifiers may be ionic or non-ionic substances. Examples of tonicity modifiers include, but are not limited to, sugars, sugar alcohols, and salts. Examples of sugars include, but are not limited to, lactose, dextrose, sucrose, glucose, and trehalose. Examples of sugar alcohols include, but are not limited to, mannitol, sorbitol, and glycerin. Examples of salts include, but are not limited to, sodium chloride, potassium chloride, sodium sulfate, and potassium phosphate. In embodiments, the tonicity modifier is a salt. In embodiments, the tonicity modifier is sodium chloride. In embodiments, the tonicity modifier comprises about 0.4 to 0.6 wt % sodium chloride. In embodiments, the tonicity modifier is a sugar. In embodiments, the tonicity modifier is a sugar and is selected from the group consisting of lactose, dextrose, sucrose, glucose, and trehalose. In embodiments, the tonicity modifier is a sugar alcohol. In embodiments, the tonicity modifier is a sugar alcohol and is selected from the group consisting of mannitol, sorbitol, glycerol, and glycerin. In embodiments, the tonicity modifier is a sugar alcohol and is mannitol or sorbitol. In embodiments, the tonicity modifier is mannitol, sorbitol, dextrose, PVP or sodium chloride. In embodiments, the tonicity modifier is mannitol. In embodiments, the tonicity modifier comprises about 4 wt % mannitol.

The vehicle and associated packaging are preferably sterilized prior to use in a patient. Various sterilization methods are available including, but not limited to, dry heat sterilization, autoclaving, e-beam, gamma, ethylene oxide, vaporized hydrogen peroxide, and supercritical carbon dioxide. The vehicle may be manufactured using aseptic processes such as filtration and packaged into sterile containers in a clean room. The vehicle may be sterilized in bulk and dispensed into sterile single dose containers aseptically. Suitable single dose containers include, but are not limited to, vials, blisters, ampoules, bottles, bags, and syringes. The vehicle may be dispensed into single dose containers and the vehicle and packaging are then terminally sterilized. The sterilization method used will depend upon the vehicle components as well as the packaging selected.

In embodiments, the vehicle comprises about 0.7 to 1.3 wt % viscosity modifier, about 0.6 wt % tonicity modifier, about 0.1 wt % surfactant, and about 0.6 wt % buffer. In embodiments, the vehicle comprises about 0.7 to 1.3 wt % viscosity modifier, about 0.6 wt % tonicity modifier, about 0.1 wt % surfactant, about 0.6 wt % buffer, and the pH is about 7.7 to 8.3. In embodiments, the vehicle comprises about 0.7 to 1.3 wt % viscosity modifier, about 0.6 wt % tonicity modifier, about 0.1 wt % surfactant, about 0.6 wt % buffer, the pH is about 7.7 to 8.3, and the viscosity is about 50 to 500 cps. In embodiments, the vehicle comprises about 0.7 to 1.3 wt % viscosity modifier, about 0.6 wt % tonicity modifier, about 0.1 wt % surfactant, about 0.6 wt % buffer, the pH is about 7.7 to 8.3, the viscosity is about 50 to 500 cps, and the vehicle is sterilized using an autoclave.

In embodiments, the vehicle comprises about 0.7 to 1.3 wt % sodium hyaluronate, about 0.6 wt % sodium chloride, about 0.1 wt % polysorbate 80, and about 0.6 wt % Tris. In embodiments, the vehicle comprises about 0.7 to 1.3 wt % sodium hyaluronate, about 0.6 wt % sodium chloride, about 0.1 wt % polysorbate 80, about 0.6 wt % Tris, and the pH is about 7.7 to 8.3. In embodiments, the vehicle comprises about 0.7 to 1.3 wt % sodium hyaluronate, about 0.6 wt % sodium chloride, about 0.1 wt % polysorbate 80, about 0.6 wt % Tris, the pH is about 7.7 to 8.3, and the viscosity is about 50 to 500 cps. In embodiments, the vehicle comprises about 0.7 to 1.3 wt % sodium hyaluronate, about 0.6 wt % sodium chloride, about 0.1 wt % polysorbate 80, about 0.6 wt % Tris, the pH is about 7.7 to 8.3, the viscosity is about 50 to 500 cps, and the vehicle is sterilized using an autoclave.

In embodiments, the vehicle comprises about 0.1 to 0.3 wt % viscosity modifier, about 4.0 wt % tonicity modifier, about 0.1 wt % surfactant, and about 0.6 wt % buffer. In embodiments, the vehicle comprises about 0.1 to 0.3 wt % viscosity modifier, about 4.0 wt % tonicity modifier, about 0.1 wt % surfactant, about 0.6 wt % buffer, and the pH is about 7.7 to 8.3. In embodiments, the vehicle comprises about 0.1 to 0.3 wt % viscosity modifier, about 4.0 wt % tonicity modifier, about 0.1 wt % surfactant, about 0.6 wt % buffer, the pH is about 7.7 to 8.3, and the viscosity is about 30 to 50 cps. In embodiments, the vehicle comprises about 0.1 to 0.3 wt % viscosity modifier, about 4.0 wt % tonicity modifier, about 0.1 wt % surfactant, about 0.6 wt % buffer, the pH is about 7.7 to 8.3, the viscosity is about 30 to 50 cps, and the vehicle is sterilized using sterile filtration.

In embodiments, the vehicle comprises about 0.1 to 0.3 wt % sodium hyaluronate, about 4.0 wt % mannitol, about 0.1 wt % polysorbate 80, and about 0.6 wt % Tris. In embodiments, the vehicle comprises about 0.1 to 0.3 wt % sodium hyaluronate, about 4.0 wt % mannitol, about 0.1 wt % polysorbate 80, about 0.6 wt % Tris, and the pH is about 7.7 to 8.3. In embodiments, the vehicle comprises about 0.1 to 0.3 wt % sodium hyaluronate, about 4.0 wt % mannitol, about 0.1 wt % polysorbate 80, about 0.6 wt % Tris, the pH is about 7.7 to 8.3, and the viscosity is about 30 to 50 cps. In embodiments, the vehicle comprises about 0.1 to 0.3 wt % sodium hyaluronate, about 4.0 wt % mannitol, about 0.1 wt % polysorbate 80, about 0.6 wt % Tris, the pH is about 7.7 to 8.3, the viscosity is about 30 to 50 cps, and the vehicle is sterilized using sterile filtration.

In alternative embodiments of the present invention, the vehicle comprises additional surfactant components. In some embodiments docusate sodium is added as a surfactant to the vehicle of the present invention. In some embodiments, sodium deoxycholate is added as a surfactant to the vehicle. In some embodiments docusate sodium and sodium deoxycholate are both added to the vehicle as a surfactant system. In some embodiments, the surfactant system includes less than about 0.015 wt % docusate sodium and less than about 0.1 wt % sodium deoxycholate. According to some embodiments including docusate sodium, an alcohol co-solvent may also be included in the vehicle. Co-solvents used with docusate sodium are selected from ethanol, benzyl alcohol, glycerin, and other appropriate alcohols. In a particular embodiment, the co-solvent is ethanol. In some embodiments utilizing ethanol as the co-solvent it may be included to less than about 5% relative to the overall vehicle. In embodiments utilizing ethanol as the co-solvent it may be included to less than about 2% relative to the overall vehicle. In embodiments utilizing ethanol as the co-solvent it may be included to less than about 1% relative to the overall vehicle. In a particular embodiment, the co-solvent is ethanol and included between about 0.1% to 0.5% relative to the overall vehicle. Importantly, the tonicity of the vehicle is adjusted to maintain an isotonic vehicle solution and the buffer is adjusted to maintain the pH appropriate for injection.

PRINT Technology

Various methods may be used to produce the drug particles. Methods include, but are not limited to, solvent casting, phase separation, interfacial methods, molding, compression molding, injection molding, extrusion, co-extrusion, heat extrusion, die cutting, heat compression, and combinations thereof. In certain embodiments, the drug particles are molded, preferably using polymeric molds.

In particular embodiments, the particles of the present disclosure are fabricated using PRINT® Technology (Liquidia Technologies, Inc., Morrisville, N.C.) particle fabrication. In particular, the particles are made by molding the materials intended to make up the particles in mold cavities.

The molds can be polymer-based molds and the mold cavities can be formed into any desired shape and dimension. Uniquely, as the particles are formed in the cavities of the mold, the particles are highly uniform with respect to shape, size, and composition. Due to the consistency among the physical and compositional makeup of the particles of the present compositions, the compositions of the present disclosure provide highly uniform release rates and dosing ranges. The methods and materials for fabricating the particles of the present disclosure are further described and disclosed in issued patents and co-pending patent applications, each of which are incorporated herein by reference in their entirety: U.S. Pat. Nos. 8,518,316; 8,444,907; 8,420,124; 8,268,446; 8,263,129; 8,158,728; 8,128,393; 7,976,759; U.S. Pat. Application Publications Nos. 2013-0249138, 2013-0241107, 2013-0228950, 2013-0202729, 2013-0011618, 2013-0256354, 2012-0189728, 2010-0003291, 2009-0165320, 2008-0131692; and pending U.S. application Ser. No. 13/852,683 filed Mar. 28, 2013 and Ser. No. 13/950,447 filed Jul. 25, 2013. In addition, the following provisional applications, each of which, are incorporated herein by reference in their entirety: 62/332,015 filed May 5, 2016; 62/440,088 filed Dec. 29, 2016; 62/442,318 filed Jan. 6, 2017; 62/463,206 filed Feb. 24, 2017; and 62/472,885 filed Mar. 17, 2017.

The mold fabricated for making the drug particles of the present invention are thin film roll-to-roll molds described in the referenced and incorporated patent art. The thin film molds include a PET backing layer having a tie-layer affixing the polymeric mold layer thereto, also as generally described in the referenced and incorporated patent art. In some embodiments, the tie-layer includes maleic anhydride.

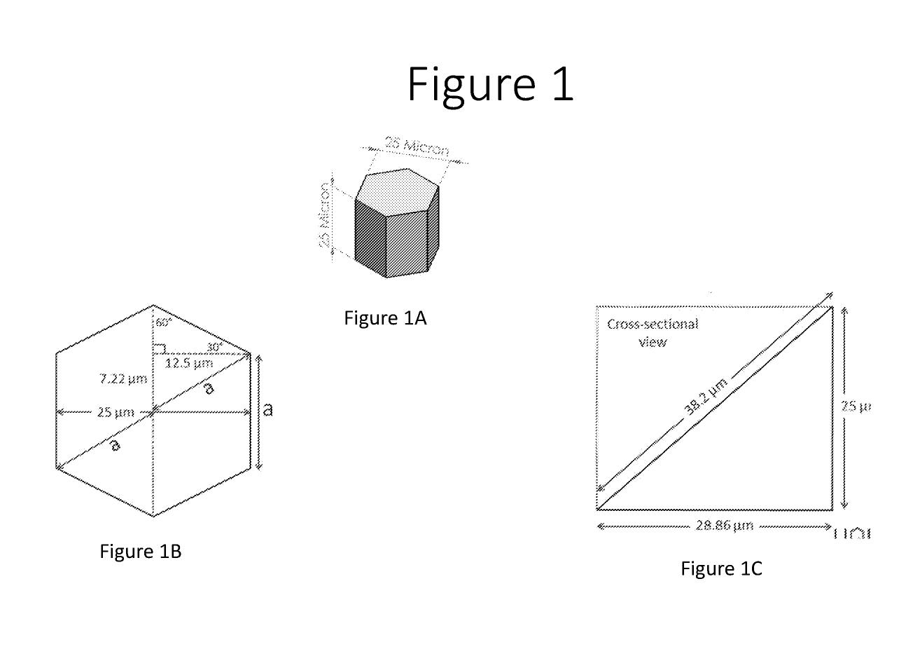

The mold cavities can be formed into various shapes and sizes. For example, the cavities may be shaped as a prism, rectangular prism, triangular prism, hexagonal prism, pyramid, square pyramid, triangular pyramid, cube, cone, cylinder, torus, or rod. The cavities within a mold may have the same shape or may have different shapes. Particles are formed within the mold cavities and the shape of the particle mimics the shape of the mold cavity. In certain aspects of the disclosure, the shapes of the particles are a prism, rectangular prism, or hexagonal prism. Prisms may be right prisms and/or regular right prisms. In a particular embodiment, the particles are right hexagonal prisms. In embodiments, the particles, in cross-section, are defined by a substantially rectangular shape, as shown in FIG. 1C.

The mold cavities can be dimensioned from nanometer to micrometer dimensions and larger. For certain embodiments of the disclosure, mold cavities are dimensioned in the nanometer and micrometer range. For example, cavities may have a dimension of between approximately 50 nanometers and approximately 100 μm. In some aspects, the mold cavity dimension may be between approximately 10 μm and approximately 50 μm. In other aspects, the mold cavity dimension may be between approximately 20 μm and approximately 30 μm. The dimension may be a largest dimension or a smallest dimension.

In some embodiments, the particles of the invention can be engineered with a specific shape and/or a specific aspect ratio. Aspect ratio refers to the ratio of the longest axis to the shortest axis of a particle. In some embodiments, particle shapes with a small surface to volume ratio are preferred. Benefits of a small surface to volume ratio include reduction in dissolution rate and improved manufacturing yield.

Once fabricated, the particles may remain on an array for storage, may remain in the mold for storage, or may be harvested immediately for storage and/or utilization. Particles may be fabricated using sterile processes, or may be sterilized after fabrication.

In one embodiment, a right hexagonal prism is fabricated with dimensions of 25 μm high×25 μm wide, wherein the width represents the distance between two vertices with an intervening vertex. FIGS. 1A, 1B and 1C depict such a hexagonal prism. The length of each side of the hexagonal face, lowercase “a” in FIG. 1B is calculated to be approximately 14.43 μm. The surface area is approximately 3,000 to 3,500 μm² and the volume is approximately 13,000 to 14,000 μm³. In some embodiments, the surface area is approximately 3,250 μm². In some embodiments, the volume is approximately 13,500 μm³. The surface area to volume ratio is calculated to be approximately 0.24. A cross-sectional view of a hexagonal prism is shown in FIG. 1C.

In some embodiments, the surface area to volume ratio is about 0.1 to 0.5. In some embodiments, the surface area to volume ratio is about 0.15 to 0.35. In some embodiments, the surface area to volume ratio is about 0.2 to 0.3.

Manufacturing Process

The process to manufacture particles generally comprises particle stock solution preparation, particle fabrication, collection, sieving, packaging, and sterilization.

The particle stock solution is prepared by dissolving the amino amide anesthetic or, if the composition also includes a polymer, the amino amide anesthetic and polymer or polymers in a suitable solvent to create a homogeneous solution. For example, acetone, methylene chloride, alcohols, acetonitrile, tetrahydrofuran, chloroform, and ethyl acetate may be used as solvents. Solvent selection will depend upon the amino amide anesthetic and polymer or polymers, if applicable, selected. Prior to use, the particle stock solution can be filtered or aseptically filtered. In embodiments, the particle stock solution comprises a 35 wt % by solids homogeneous solution of 40-50 wt % PLGA and 50-60 wt % bupivacaine free base in acetone. In embodiments having no polymer component, the particle stock solution comprises a 40 wt % by solids homogeneous solution of 100 wt % bupivacaine free base in methylene chloride. In embodiments, the particle stock solution is filtered through a 0.2 μm filter.

To fabricate particles, the particle stock solution may be dispensed directly into a mold. Alternatively, the particle stock solution may be applied to a film, dried, and transferred into a mold using heat and/or pressure. After molding, particles may be maintained in the mold, may be removed from the mold and maintained on a film, or be maintained on the film and in the mold. In embodiments, the particle stock solution is applied to a PET film pre-coated with a PVOH harvest layer and dried. After drying, the dried film on the PET film is mated to a polymer mold having cavities of shape and size of the desired particles and run through a nip point in a roll-to-roll laminator which transfers the dried particle stock solution film into the mold cavities. In particular embodiments, the dried film is transferred into 25 μm hexagon mold cavities to fabricate a plurality of 25 um particles of the present invention.

After fabrication, the particles may be stored for a time period prior to harvesting in an annealing process while still in the mold cavities. Storage may be under ambient conditions or at an elevated temperature. For example, storage may be at 25° C. and 25% relative humidity. Storage may be at 40° C. and 25% relative humidity. Storage may be at ambient conditions such as 20-25° C. and 5-60% relative humidity. Storage may be for hours, days, weeks, or months. Storage may be for at least 1, 2, 3, 4, 5, 6, 7, 8, 9, 10, 11, 12, 13, 14, 15, 16, 17, 18, 19, 20, 21, 22, 23, or 24 hours. Storage may be for at least 1, 2, 3, 4, 5, 6, 7, 8, 9, 10, or more days. Storage may be for 1, 2, 3, 4, 5, 6, 7, 8, 9, 10, 11, or 12 months or more. In embodiments, storage is for at least about 10 days. In embodiments, storage is for about 10-14 days. In embodiments, particles are not stored prior to harvesting. In embodiments, particles are stored for at least about 10 days at 40° C. and 25% relative humidity. In embodiments, particles are stored for at least about 10 days at 25° C. and 25% relative humidity. In embodiments, particles are stored for at least about 10 days under ambient conditions (i.e. 20-25° C. and 5-60% relative humidity).

After storage or after fabrication without intervening storage, the particles are harvested. During harvesting, the particles are removed from the mold, the film, and/or the mold and the film. In some embodiments, the harvesting includes a process selected from the group including mechanical harvesting or dissolution harvesting.

In dissolution harvesting processes, a liquid is used to collect the particles. The particles are harvested from a dissolvable substrate, sheet, or film. The dissolvable substrate, sheet, or film may have been used in the fabrication process or the dissolvable substrate, sheet, or film may have been applied to the particles after the fabrication process. The dissolvable substrate can include, but are not limited to pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, combinations thereof, and the like. For example, if particles are on a polyvinyl alcohol film, the polyvinyl alcohol film may be dissolved using water and the particles collected using filtration.

In mechanical harvesting processes, the particles are harvested using a mechanical force such as scraping, brushing and the like. For example, particles may be removed from a film by scraping with a blade.

After harvesting, particles may be bulk packaged or may be packaged into single dose containers. If bulk packaged, the particles may be stored at −20° C. in Tyvek bags with a foil overwrap and dessicant. Particles may also be dispensed into single dose containers and stored. Suitable containers include those made of type 1 tubing class. Suitable single dose containers include, but are not limited to, vials, blisters, ampoules, bottles, bags, and syringes. Once packaged into single dose containers, preferably the particles and associated packaging are sterilized prior to use in a patient. The particles may be manufactured using sterile processes such as ascetic filtration and fabrication and packaging in a clean room. The particles may be sterilized in bulk and dispensed into sterile single dose containers aseptically. The particles may be dispensed into single dose containers, joined with a vehicle, packaged into a kit, and are then terminally sterilized. The sterilization method used will depend upon the particle components as well as the packaging selected. Various sterilization methods are available including, but not limited to, dry heat sterilization, autoclaving, e-beam, gamma, ethylene oxide, vaporized hydrogen peroxide, and supercritical carbon dioxide.

Use of Materials

The drug particles and/or particles dispersed in vehicle may be used to induce analgesia in a patient. The compositions may be delivered as drug particles and/or as drug particles suspended in a vehicle. Delivery may, for example, be topical, direct application to a site of need, injection, parenteral or via infiltration. A syringe, cannula, trocar, or other device containing a lumen may be used to deliver via injection or infiltration. For topical delivery the drug particles may be applied using any number of methods, including but not limited to, painting, swabbing, dabbing. The drug particles may be used before, during, or after a surgical procedure. For example, the drug particles may be used for single-dose infiltration into a surgical site to produce post-operative analgesia. In certain embodiments, the drug particles may be used with or without resuspension in the vehicle and directly applied to a site of need. Examples of direct application can include direct application to a surgical site or packing the drug particle powder into a site of need, such as, for example, post dental extraction or other dental procedures, biopsy, injury or treatment site. The drug particles may be administered during surgery at any time. Preferably, the drug particles are administered at or near the conclusion of the surgical procedure, at or near the time of the closure of any surgical incisions.

The dose may be adjusted by using more or less drug particles of a given composition or by increasing or decreasing the amino amide content in the drug particles. For example, to deliver 200 mg of bupivacaine, 10 mL of a 20 mg/mL suspension containing particles containing 100 wt % bupivacaine or 20 mL of a 20 mg/mL suspension containing drug particles containing about 50 wt % bupivacaine may be used.

According to an embodiment of the present invention, because the drug particle are fabricated in mold cavities and, therefore, take on the controlled shape and size of the mold cavity, the particles are particularly suitable for aerosolization delivery to a site in need of action. For example, the particles are fabricated of a shape and size that provide applicable aerodynamic properties such as, for example, low particle-particle interaction forces, preferred aerodynamic size and shape and the like. The particles are loaded into an aerosolization delivery device, such as a device for disturbing resting particles into an air suspension and pumping the particle through a nozzle to a delivery site. The particle can, in the alternative, be loaded into a metered dose aerosolization device that is designed to provide a controlled delivery volume of aerosol and/or particles in response to a triggering event. Accordingly, the particles are aerosolized and deposited in an even distribution manner on the surface of a site for delivery. According to such embodiments, a method of using the aerosolization delivery includes dispensing the drug particles from the aerosolization device onto or into a site in need thereof. In another embodiment, the drug particles can be included in a foaming deposition that provides foam to a site in need thereof that contains the particles.

Kits

Particles and/or vehicles may be packaged into kits for ease of use. Packaging serves several purposes: to contain each kit component and to keep it separated from other components, to protect the kit components, to present the kit components to the end user, to inform the end user, to identify the individual kit components and/or system, and for convenient presentation to the end user.

For example, a kit may contain drug particles and vehicle packaged in separate vials. A kit may contain one particle vial and one vehicle vial. A kit may contain more than one particle vial and one vehicle vial. A kit may contain more than one particle vial and more than one vehicle vial. The particle and vehicle vial(s) may be packaged prior to sterilization and terminally sterilized using known sterilization methods. The particles may be manufactured and packaged using sterile methods. The vehicle may be manufactured and packaged using sterile methods. The particles and vehicle may then be kitted together and terminally sterilized.

The kit may contain ancillary items. For example, the kit may contain ancillary items to aid in suspending the drug particles in the vehicle. Some ancillary items to aid in suspending the drug particles include, but are not limited to, vials, stir bars, shakers, mixers, sonicators, adaptors, connectors, and vortexers. The kit may contain ancillary items to aid in application of the drug particles or the drug particles suspended in vehicle to the site of interest. Examples include, but are not limited to, syringes, needles, cannulas, trocars, devices containing a lumen, brushes, swabs, daubers, rollers, and sponges. If delivery by aerosolization is desired, the kit may contain devices to disturb the resting particles into an air suspension. Such devices include, but are not limited to, pumps, devices with nozzles, metered dose devices, and dry powder devices. Drug particles may also be applied contained in foam or paste. Such kits may contain foams and/or foaming devices to combine with the drug particles.

Kit components may be sterilized individually and/or manufactured using sterile processes prior to kitting and terminally sterilized once the kit is assembled. Some kit components may be sterilized individually and/or manufactured using sterile processes prior to kitting. Some kit components may not be sterile prior to kitting. Kit components previously sterilized and/or manufactured using sterile processes may be combined with non-sterile kit components and the combined components are then terminally sterilized.

Kit components may be sterilized once in their single dose containers, or may be sterilized in its final packaging. Final packaging may include components such as pouches, overwrap, shrink wrap, desiccants, trays, cartons, and boxes. Additional materials included may include gel packs, temperature monitoring devices, crush-resistant packaging, labels, instructions, and shipping containers.

Once individual kits are assembled, they may be combined with other kits into other packaging, such as boxes or crates, for storage and/or sale.

Suspension of Particles in Vehicle

Drug particles may be suspended in vehicle for application by the end user. Drug particles may be suspended or combined with the vehicle prior or just prior to use by the end user. Drug particles may be combined with the vehicle using any number of processes. Drug particles may be added to the vehicle in the vehicle's container or the vehicle may be added to the drug particles in the drug particles' container. Alternatively, the drug particles and vehicle may be combined using an additional container. Drug particles may be mixed, vortexed, sonicated, shaken, turned end over end, stirred, swirled, orbitally swirled, or ultrasonicated. In alternative embodiments, the drug particles may be combined with the vehicle through a dual-chambered syringe or a self-mixing syringe just prior to or in conjunction with dispensing, application and/or use.

In some embodiments, the drug particles are mixed into or with the vehicle to prepare for injection according to the following mixing procedure:
Vortex room temperature vial(s) of drug particles to disrupt any potential aggregates. For example, the drug particle containers can be vortexed for approximately 2 minutes.
Transfer the volume of vehicle to obtain the desired drug particle concentration into the drug particle container. For example, a needle may be used to draw the vehicle up into a syringe.
Alternately vortex and sonicate the container holding the drug particles and vehicle until the suspension appears uniform, i.e. no clumps observed. For example, the container holding the drug particles and vehicle may be vortexed for 15 seconds followed by sonication for 15 seconds. This pattern of vortex and sonicate, may be repeated for a number of cycles, for example, up to nine.
Vortex the container one last time prior to drawing the suspension volume up into the delivery device, i.e. syringe. Invert the container holding the suspension and draw up the desired volume. For example, draw up the suspension using a needle into a syringe.
To further mix the suspension, attach an adaptor and a second delivery device (i.e. syringe). Mix the suspension by transferring it from delivery device to delivery device a number of times. Visually confirm the suspension is homogeneous. For example, attach a two-way syringe to syringe adaptor to a syringe containing the vortexed/sonicated suspension. Attach another syringe to the other side of the adaptor. Mix the suspension by plunging it through the adaptor into the opposite syringe a total of ten times in each direction with each plunge taking approximately two seconds.
Disconnect the delivery device from the adaptor and attach a dispensing device. Remove any air and administer the suspension to the area of interest. If administration of the suspension does not occur in approximately one minute, repeat one or more of the mixing steps above. If administration of the suspension does not occur in approximately 30 minutes, discard. For example, disconnect the syringe containing the suspension from the adaptor. Attach a needle and administer the suspension to the area of interest.

In alternative embodiments of the present invention, drug particles containing an amino amide anesthetic are suspended with a vehicle containing about 0.7 to 1.3 wt % viscosity modifier, about 0.6 wt % tonicity modifier, about 0.1 wt % surfactant, about 0.6 wt % buffer, a pH of about 7.7 to 8.3, and viscosity of about 50 to 500 cps using the above procedure. In alternative embodiments, drug particles containing an amino amide anesthetic are suspended with a vehicle containing about 0.7 to 1.3 wt % sodium hyaluronate, about 0.6 wt % sodium chloride, about 0.1 wt % polysorbate 80, about 0.6 wt % Tris, a pH of about 7.7 to 8.3, and a viscosity of about 50 to 500 cps. In embodiments, drug particles containing bupivacaine free base are suspended with a vehicle containing about 0.7 to 1.3 wt % viscosity modifier, about 0.6 wt % tonicity modifier, about 0.1 wt % surfactant, about 0.6 wt % buffer, a pH of about 7.7 to 8.3, and viscosity of about 50 to 500 cps using the above procedure. In embodiments, drug particles containing bupivacaine free base are suspended with a vehicle containing about 0.7 to 1.3 wt % sodium hyaluronate, about 0.6 wt % sodium chloride, about 0.1 wt % polysorbate 80, about 0.6 wt % Tris, a pH of about 7.7 to 8.3, and a viscosity of about 50 to 500 cps.

In embodiments, drug particles containing an amino amide anesthetic and a biocompatible polymer are suspended with a vehicle containing about 0.7 to 1.3 wt % viscosity modifier, about 0.6 wt % tonicity modifier, about 0.1 wt % surfactant, about 0.6 wt % buffer, a pH of about 7.7 to 8.3, and viscosity of about 50 to 500 cps using the above procedure. In embodiments, drug particles containing bupivacaine free base and a biocompatible polymer are suspended with a vehicle containing about 0.7 to 1.3 wt % viscosity modifier, about 0.6 wt % tonicity modifier, about 0.1 wt % surfactant, about 0.6 wt % buffer, a pH of about 7.7 to 8.3, and viscosity of about 50 to 500 cps using the above procedure. In embodiments, drug particles containing bupivacaine free base and PLGA are suspended with a vehicle containing about 0.7 to 1.3 wt % viscosity modifier, about 0.6 wt % tonicity modifier, about 0.1 wt % surfactant, about 0.6 wt % buffer, a pH of about 7.7 to 8.3, and viscosity of about 50 to 500 cps using the above procedure.

In embodiments, drug particles containing an amino amide anesthetic and a biocompatible polymer are suspended with a vehicle containing about 0.7 to 1.3 wt % sodium hyaluronate, about 0.6 wt % sodium chloride, about 0.1 wt % polysorbate 80, about 0.6 wt % Tris, a pH of about 7.7 to 8.3, and a viscosity of about 50 to 500 cps. In embodiments, drug particles containing bupivacaine free base and a biocompatible polymer are suspended with a vehicle containing about 0.7 to 1.3 wt % sodium hyaluronate, about 0.6 wt % sodium chloride, about 0.1 wt % polysorbate 80, about 0.6 wt % Tris, a pH of about 7.7 to 8.3, and a viscosity of about 50 to 500 cps. In embodiments, drug particles containing bupivacaine free base and PLGA are suspended with a vehicle containing about 0.7 to 1.3 wt % sodium hyaluronate, about 0.6 wt % sodium chloride, about 0.1 wt % polysorbate 80, about 0.6 wt % Tris, a pH of about 7.7 to 8.3, and a viscosity of about 50 to 500 cps.

In embodiments according to the vehicle below, the drug particles are mixed with the vehicle according to the following steps:
Tap room temperature vial(s) of drug particles against a firm surface to disrupt any potential aggregates.
Transfer the volume of vehicle to obtain the desired drug particle concentration into the drug particle container. For example, a needle may be used to draw the vehicle up into a syringe.
Gently swirl the container holding the drug particles and vehicle until a homogenous suspension is observed. For example, swirl the container 30 second intervals until a homogeneous suspension is observed.
Draw up the desired volume of suspension into the delivery device and administer to the area of interest. If the suspension is not drawn up into the delivery device within one minute, repeat the swirling above. If administration of the suspension does not occur in approximately 30 minutes, discard and prepare a new mixture.

According to certain preferred embodiments, the above mixing steps utilize the following vehicle compositions with the PLGA/bupivacaine drug particles described and disclosed herein:

Drug particles of the present invention containing an amino amide anesthetic and a biocompatible polymer are suspended with a vehicle containing about 0.1 to 0.3 wt % viscosity modifier, about 4.0 wt % tonicity modifier, about 0.1 wt % surfactant, about 0.6 wt % buffer, a pH of about 7.7 to 8.3, and viscosity of about 30 to 50 cps using the above procedure. In embodiments, drug particles containing bupivacaine free base and a biocompatible polymer are suspended with a vehicle containing about 0.1 to 0.3 wt % viscosity modifier, about 4.0 wt % tonicity modifier, about 0.1 wt % surfactant, about 0.6 wt % buffer, a pH of about 7.7 to 8.3, and viscosity of about 30 to 50 cps using the above procedure. In embodiments, drug particles containing bupivacaine free base and PLGA are suspended with a vehicle containing about 0.1 to 0.3 wt % viscosity modifier, about 4.0 wt % tonicity modifier, about 0.1 wt % surfactant, about 0.6 wt % buffer, a pH of about 7.7 to 8.3, and viscosity of about 30 to 50 cps using the above procedure.

Drug particles of the present invention containing an amino amide anesthetic and a biocompatible polymer are suspended with a vehicle containing about 0.1 to 0.3 wt % sodium hyaluronate, about 4.0 wt % mannitol, about 0.1 wt % polysorbate 80, about 0.6 wt % Tris, a pH of about 7.7 to 8.3, and a viscosity of about 30 to 50 cps. In embodiments, drug particles containing bupivacaine free base and a biocompatible polymer are suspended with a vehicle containing about 0.1 to 0.3 wt % sodium hyaluronate, about 4.0 wt % mannitol, about 0.1 wt % polysorbate 80, about 0.6 wt % Tris, a pH of about 7.7 to 8.3, and a viscosity of about 30 to 50 cps. In embodiments, drug particles containing bupivacaine free base and PLGA are suspended with a vehicle containing about 0.1 to 0.3 wt % sodium hyaluronate, about 4.0 wt % mannitol, about 0.1 wt % polysorbate 80, about 0.6 wt % Tris, a pH of about 7.7 to 8.3, and a viscosity of about 30 to 50 cps.

In alternative embodiments of the present invention, the vehicle comprises additional surfactant components. In some embodiments docusate sodium is added as a surfactant to the vehicle of the present invention. In some embodiments, sodium deoxycholate is added as a surfactant to the vehicle. In some embodiments docusate sodium and sodium deoxycholate are both added to the vehicle as a surfactant system. In some embodiments, the surfactant system includes less than about 0.015% docusate sodium and less than about 0.1% sodium deoxycholate. According to some embodiments including docusate sodium, an alcohol co-solvent may also be included in the vehicle. Co-solvents used with docusate sodium are selected from ethanol, benzyl alcohol, glycerin, and other appropriate alcohols. In a particular embodiment, the co-solvent is ethanol. In some embodiments utilizing ethanol as the co-solvent it may be included to less than about 5% relevant to the overall vehicle. In some embodiments utilizing ethanol as the co-solvent it may be included to less than about 2% relevant to the overall vehicle. In some embodiments utilizing ethanol as the co-solvent it may be included to less than about 1% relevant to the overall vehicle. In a particular embodiment, the co-solvent is ethanol and included between about 0.1% to 0.5% relevant to the overall vehicle. Importantly, the tonicity of the vehicle is adjusted to maintain an isotonic vehicle solution and the buffer is adjusted to maintain the pH appropriate for injection.

Excipients and their particular function in the vehicle of the present invention are included in the following table. In particular, the excipients can be included in the vehicle of the present invention to provide wettability to the drug particles to enhance suspendability of the drug particles in the vehicle and/or dispersability to enhance fine dispersion of the drug particles suspended in the vehicle.

| Excipient | Function |
|---|---|
| T-Butanol | Wettability/Dispersability |
| N,N-dimethylacetamide | Wettability/Dispersability |
| DMSO | Wettability/Dispersability |
| Sorbitol | Wettability/Dispersability |
| Mannitol | Wettability/Dispersability |
| Povidone | Wettability/Dispersability |
| Niacinamide | Wettability/Dispersability |
| Propylene glycol | Wettability/Dispersability |
| PEG 400 | Wettability/Dispersability |
| PEG 300 | Wettability/Dispersability |
| PEG200 | Wettability/Dispersability |
| N-Methyl-2-Pyrrolidone | Wettability/Dispersability |
| Glycerin | Wettability/Dispersability |
| EtOH/Ethanol | Wettability/Dispersability |
| BzOH/Benzyl alcohol | Wettability/Dispersability |
| PEG 4000 | Wettability/Dispersability |
| PEG 3350 | Wettability/Dispersability |
| Benzyl Benzoate | Wettability/Dispersability |
| N-acetyl tryptophan | Wettability/Dispersability |
| Diethanolamine | Wettability/Dispersability |
| Ethanolamine HCl | Wettability/Dispersability |
| Tricaprylin (MCT) | Wettability/Dispersability |
| Medium chain triglycerides | Wettability/Dispersability |
| Sorbitan monopalmitate | Wettability/Dispersability |
| Sorbitan monopalmitate | Wettability/Dispersability |
| Sorbitan monolaurate | Wettability/Dispersability |
| Sodium deoxycholate | Wettability/Dispersability |
| Simethicone | Wettability/Dispersability |
| PvOH/Polyvinyl alcohol | Wettability/Dispersability |
| Polysorbate-80 | Wettability/Dispersability |
| Polysorbate 40 | Wettability/Dispersability |
| Polysorbate 20 | Wettability/Dispersability |
| Polyoxyethylated fatty acid esters | Wettability/Dispersability |
| Poloxamer 188 | Wettability/Dispersability |
| PVP | Wettability/Dispersability |
| PEG-20 Sorbitan Isostearate | Wettability/Dispersability |
| PEG 60 Castor Oil - Cremophor RH 60 | Wettability/Dispersability |
| PEG 40 Castor Oil | Wettability/Dispersability |
| PEG 35 Castor Oil (cremophor EL) | Wettability/Dispersability |
| Lecithin | Wettability/Dispersability |
| Glyceryl Trioleate | Wettability/Dispersability |
| DSPE-PEG | Wettability/Dispersability |
| Docusate Sodium | Wettability/Dispersability |
| Cholesterol | Wettability/Dispersability |
| Specific phospholipids: | Wettability/Dispersability |
| DSPE, GMPG, DMPC, DOPC, | |
| DPPG and the like | |

Other excipients that may be included in the vehicle of the present invention are included in the following table.

Excipient

Monothioglycerol
Gentisic acid ethanolamine
Alpha tocopherol
Propyl gallate
Phenol
Methyl paraben
M-cresol
Chlorobutanol
Butylparaben
Benzalkonium Chloride
phenylethyl alcohol
Benzyl Benzoate

Crystallized Drug Product

In some embodiments of the present invention, the active agent is dissolved into a solvent and introduced into the molds described herein but without any polymer matrix material or other excipients present. In such embodiments, the particles formed from the PRINT process comprise nearly 100 percent pure crystallized drug substance. As such, these crystal drug particles are stable and provide advantageous storage, handling, delivery, and performance features. For example, the crystal particles of the present invention provide avoidance of introducing a polymer or other matrix (lipid, or the like) material to a patient. In some embodiments, the avoidance of matrix material can increase drug performance, reduce tissue irritation, inflammation, reaction or damage, reduce drug particle interactions, increase dosage per unit volume of vehicle, and combinations thereof.

In some embodiments, a composition of the present invention includes a plurality of particles, each particle of the plurality defined by a non-spherical shape having a broadest liner dimension not more or less than 1 micrometer from a predetermined broadest linear dimension; and wherein each particle comprises an amino amide anesthetic or a pharmaceutically acceptable salt, hydrate, and/or solvate thereof and optionally PLA and/or PLGA polymer.

In another embodiment of the present invention, the composition of the particle includes a plurality of particles, each particle of the plurality defined by a non-spherical shape defined, in cross-section by, a substantially rectangular shape, wherein each particle comprises an amide anesthetic or a pharmaceutically acceptable salt, hydrate, and/or solvate thereof and optionally PLA and/or PLGA polymer, and the composition includes a vehicle comprising a viscosity modifier, a surfactant, a buffer, and, optionally, a tonicity modifier.

According to an aspect of the invention, the vehicle the particles are suspended in before injection comprises an aqueous vehicle of sodium hyaluronate, sodium chloride, Tris base, Tris HCl and optionally polysorbate 80, where the sodium hyaluronate has an inherent viscosity of 1.6-2.2 m³/kg and comprises 7.0-10.0 mg/mL of vehicle.

According to other embodiments, drug particles may be fabricated as crystalline by controlled crystallization, crystallization from solution, templated crystallization, or solid phase crystallization and processes including continuous crystallization and batch crystallization. In other embodiments, larger crystalline drug bulk materials may be reduced to crystalline particles applicable to the present invention similar or in place of the molded particles by micronization, milling, grinding, spray-drying, or wet polishing.

Surgical Procedures for Application of the Present Invention

The drug particles of the invention may be utilized in a variety of surgical procedures to produce extended-release analgesia over a 3 to 5 day post-surgical period. Surgical procedures applicable for the present invention particles may be laparoscopic, minimally invasive procedures or may be open. The drug particles may be used in soft tissue, orthopedic, spinal surgeries or otherwise as determined by a medical professional.

Minimally invasive procedures include, but are not limited to, joint arthroscopic, laparoscopic, mediastinoscopic, thoracoscopic, cholecystectomy, appendectomy, gastroenterostomy, hemicolectomy, sigmoidectomy, including some valve replacement procedures by cardiologists, certain discectomies or other similar percutaneous procedures by neurosurgeons and orthopedic surgeons and other percutaneous procedures. Examples of soft tissue surgeries include, but are not limited to, abdominal, anorectal, breast, reconstructive, female and male genitourinary, colorectal, transversus abdominis plane (TAP) block-based procedures, colorectal, and/or plastic surgeries. Examples of abdominal surgeries include, but are not limited to, hernia, bariatric, gastric sleeve, gastrectomy, ileostomy, open and laparoscopic colorectal, ileostomy reversal, and/or abdominal wall reconstruction. An example of breast surgery includes, but is not limited to, mastectomy. An example of reconstructive surgery includes, but is not limited to, plastic reconstructive surgery. Examples of female genitourinary surgeries include, but are not limited to, hysterectomy, episiotomy, obstetric laceration repair, low cervical caesarian section, and caesarian section. An example of male genitourinary surgery includes, but is not limited to, prostatectomy. Examples of colorectal surgeries include, but are not limited to, hemorrhoidectomy, rectal resection, hemicolectomy, sigmoidectomy, bowel resection (small or large), and colectomy. Examples of plastic surgeries include, but are not limited to, breast augmentation, breast reduction, and/or abdominoplasty.

Examples of orthopedic surgeries include, but are not limited to, bunionectomy, knee arthroplasty, total knee replacement, hip arthroplasty, total hip replacement, shoulder arthroplasty, total shoulder replacement, foot and ankle surgeries, fusions and/or repair of fractures.

Spinal surgeries may take place in the cervical, thoracic, lumbar, and/or sacral regions. Examples of spinal surgeries include, but are not limited to, fusions, discectomy, discotomy, sacral fusion, lumbar fusion, and/or posterior cervical fusion.

### EXAMPLES

### Example 1

Manufacture of Particles

### Example 1A: Manufacture of Bupivacaine/PLGA Particles

Particles containing bupivacaine free base and PLGA were manufactured. First, a particle stock solution was prepared. A 35 wt % solids homogeneous solution of 40 wt % PLGA (Evonik Industries, Resomer RG502H) and 60 wt % bupivacaine free base (Cayman Chemical Company) in acetone was prepared. The solution was filtered through a sterile 0.2 μm PTFE filter. The resulting particle stock solution was cast at room temperature onto a 4 mil PET film pre-coated with a polyvinyl alcohol harvest layer.

To form particles, the dried cast film was laminated against a 25 μm hexagon mold (Liquidia Technologies, Inc. Morrisville, N.C.). The mold/film was passed through a laminator at 280° F. at 5 feet/minute.

Annealing the particles in the mold provides crystallization of the amino amide anesthetic and occurs over about 9-13 days at a temperature of 40° C. and a 10-25% RH. In alternative embodiments, drug particles were stored in the mold at ambient conditions for approximately 20 days prior to harvesting and a second portion of particles were stored while in the mold at 40° C./25% relative humidity for approximately 20 days prior to harvesting. After storage and/or annealing, the harvest layer, with drug particles attached, was removed from the mold. Particles were scraped from the harvest layer using a doctor blade under ambient conditions. The recovered drug particles were passed through 250 μm and 106 μm sieves to thoroughly mix the particles and remove any large size impurities. The drug particles were dried, under nitrogen, at 25° C. under vacuum. After drying, the particles were aliquoted into vials and were sterilized using gamma radiation. The sterilized particles were stored at −20° C. until use.

### Example 1B: Manufacture of Bupivacaine Particles

Particles containing bupivacaine free base were manufactured. Particles were manufactured as above in Example 1A with the following modifications. A 40 wt % solids homogeneous solution of 100 wt % bupivacaine free base (Cayman Chemical Company) in methylene chloride was used. For these particles, the laminator was at 300° F. In some embodiments, particles were stored under annealing conditions while in the mold at ambient conditions for approximately 20 days prior to harvesting. In other embodiments, particles were stored in the molds under annealing conditions to allow for crystallization in the mold at about 9-13 days at ambient temperature and relative humidity (19-25° C. & 20-40% RH).

### Example 1C: Manufacture of Levobupivacaine Particles

Particles containing levobupivacaine free base were manufactured. Particles were manufactured as above in Example 1B with the following modifications. A 40 wt % solids homogeneous solution of 100 wt % levobupivacaine free base (BOC Sciences) in methylene chloride was prepared was used. For these particles, the laminator was at 320° F. Particles were harvested immediately without intervening annealing or storage.

### Example 2

Manufacture of Vehicle

30 mg of polysorbate 80 (NOF, HX2) was measured and placed into a clean tared beaker. A small volume of water for injection (WFI) was added and the polysorbate 80 and water were stirred until visually dissolved. 291 mg Tris-base (JT Baker, 4109-01) and 567 mg Tris-HCl (JT Baker, 4106-01) were weighed and transferred into a clean tared vessel. The dissolved polysorbate 80 solution was added to the powders and the remaining water (total water added 28.962 g) was added. The solution was stirred. While stirring, 150 mg sodium hyaluronate (Stanford Chemical, HA-EP-1.8) was added slowly to avoid the formation of undissolved clumps. After addition of sodium hyaluronate, the solution was stirred at room temperature until homogeneous. The solution was transferred into a glass bottle for sterilization. The solution was sterilized at 121° C. for twenty minutes. The solution was cooled to room temperature.

### Example 3

Stability of Particles

Particles from Example 1A (stored at 40° C. for 19 days and sterilized using 25 kGy gamma irradiation) and Example 1B (stored at ambient conditions for 19 days and sterilized using 25 kGy gamma irradiation) were aliquoted into vials. For Example 1A 400+/−8 mg was aliquoted into 10 mL type 1 tubing glass vials which were backfilled with nitrogen, stoppered, and crimped. For Example 1B, 200+/−6 mg was aliquoted into 10 mL type 1 tubing glass vials which were backfilled with nitrogen, stoppered, and crimped. After the particles are sealed in the vials under nitrogen they are gamma irradiated for sterilization.

Vials were placed under three storage conditions: −20° C., 2-8° C., and 25° C./60% relative humidity. Particles were analyzed at T=0, 1 month, 2 months, 3 months, 6 months, and 9 months for particle size and bupivacaine content.

Particle Size by Laser Diffraction Using Malvern Mastersizer 3000

A 0.05-0.2M sodium carbonate-bicarbonate buffer, pH 9.4-9.6 was prepared. Polysorbate 80 solution was added to 0.1% (v/v) in the bicarbonate buffer to form the aqueous dispersant.

Approximately 40-60 mg particles were transferred into a 2 mL vial. Using a transfer pipet, approximately 2 mL dispersant was added to the particles in the vial. The particle/dispersant suspension was vortexed for approximately 10 seconds. Using a transfer pipet, the particle/dispersant suspension was added dropwise into the Hydro-MV of the Malvern Mastersizer 3000 for analysis.

For the instrument parameters, the stir rate was 2,000 rpm. The measurement mode was Fraunhofer approximation. The background collection duration was 10 seconds. For sample data collection, the measurement duration was 20 seconds with at least five repeat measurements. For sample data collection, the sample was sonicated for 1 minute at 10% and sonication was turned off prior to data collection. The obscuration target was 5-7% and the data analysis was the General Purpose model.

T=0 data was collected as above with the following modifications. For the aqueous dispersant, a 0.02% (v/v) polysorbate bicarbonate buffer pH 9.4-9.6 was used. The sample was sonicated at 5% throughout data collection. Data analysis was performed with the Narrow Mode model. Results for T=1 month, 2 months, and later months may differ slightly from initial T=0 months results due to modifications in the analysis method.

Data collected was D10, D50, and D90. Results for the drug particles of Example 1A are shown in the table below. Drug particles of Example 1A are physically stable at −20° C. and 2-8° C. for at least 9 months.

| Time | Storage | | | | | | |
|---|---|---|---|---|---|---|---|
| Interval | Condition | D10 (μm) | | D50 (μm) | | D90 (μm) | |
| (months) | (° C.) | AVE | STDEV | AVE | STDEV | AVE | STDEV |
| 0 | NA | 20.0 | 0.8 | 30.3 | 1.0 | 42.0 | 1.9 |
| 1 | −20 | 20.8 | 0.2 | 27.6 | 0.2 | 36.7 | 0.3 |
| | 2-8 | 20.9 | 0.1 | 27.7 | 0.2 | 36.7 | 0.3 |
| | 25 | 21.0 | 0.1 | 28.0 | 0.2 | 37.3 | 0.5 |
| 2 | −20 | 20.8 | 0.1 | 27.6 | 0.1 | 36.6 | 0.3 |
| | 2-8 | 20.9 | 0.1 | 27.6 | 0.1 | 36.4 | 0.3 |
| | 25 | 21.4 | 0.0 | 28.4 | 0.2 | 37.3 | 0.5 |
| 3 | −20 | 19.8 | 0.1 | 26.1 | 0.3 | 34.0 | 0.4 |
| | 2-8 | 19.7 | 0.8 | 26.1 | 0.6 | 36.8 | 2.7 |
| | 25 | 20.4 | 0.3 | 27.4 | 0.7 | 38.4 | 6.0 |
| 6 | −20 | 15.1 | 0.4 | 22.3 | 0.5 | 32.1 | 0.8 |
| | 2-8 | 18.5 | 0.4 | 28.4 | 0.3 | 42.8 | 0.5 |
| | 25 | 13.7 | 4.1 | 28.7 | 0.4 | 45.0 | 0.6 |
| 9 | −20 | 20.3 | 0.1 | 27.5 | 0.1 | 36.9 | 0.4 |
| | 2-8 | 20.9 | 0.3 | 27.5 | 0.1 | 36.4 | 0.5 |
| | 25 | 20.7 | 0.2 | 29.0 | 0.3 | 40.4 | 0.9 |

Results for the drug particles of Example 1B are shown in the table below. Drug particles of Example 1B are physically stable at −20° C. and 2-8° C. for at least 9 months.

| Time | Storage | | | | | | |
|---|---|---|---|---|---|---|---|
| Interval | Condition | D10 (μm) | | D50 (μm) | | D90 (μm) | |
| (months) | (° C.) | AVE | STDEV | AVE | STDEV | AVE | STDEV |
| 0 | NA | 16.7 | 0.4 | 27.0 | 0.4 | 38.7 | 1.0 |
| 1 | −20 | 18.3 | 0.1 | 26.4 | 0.0 | 37.4 | 0.1 |
| | 2-8 | 19.2 | 0.4 | 26.6 | 0.5 | 36.7 | 0.8 |
| | 25 | 10.0 | 2.8 | 29.2 | 0.2 | 49.7 | 1.2 |
| 2 | −20 | 19.3 | 0.1 | 26.8 | 0.3 | 37.2 | 0.9 |
| | 2-8 | 19.7 | 0.1 | 27.8 | 0.0 | 38.2 | 0.1 |
| | 25 | 3.9 | 0.2 | 25.9 | 1.2 | 651.0 | 585.0 |
| 3 | −20 | 18.8 | 0.2 | 25.2 | 0.3 | 33.6 | 0.3 |
| | 2-8 | 19.0 | 0.1 | 28.2 | 0.9 | 41.3 | 2.7 |
| | 25 | 8.7 | 3.4 | 30.2 | 4.7 | 308.0 | 564.0 |
| 6 | −20 | 21.0 | 0.1 | 27.2 | 0.1 | 34.8 | 0.3 |
| | 2-8 | 19.8 | 0.3 | 27.0 | 0.1 | 36.0 | 0.6 |
| | 25 | 19.2 | 1.1 | 26.9 | 0.8 | 37.1 | 0.8 |
| 9 | −20 | 18.1 | 0.7 | 24.8 | 0.5 | 33.7 | 0.5 |
| | 2-8 | 18.6 | 0.4 | 29.8 | 1.0 | 47.4 | 3.9 |
| | 25 | 15.4 | 3.1 | 25.8 | 3.6 | 1280.0 | 689.0 |

Bupivacaine Content Via HPLC

Bupivacaine content was determined using HPLC using the chromatography parameters detailed in the table below—Chromatography Parameters for Bupivacaine Determination

| Flow rate | 1.0 mL/minute | | |
|---|---|---|---|
| Injection volume | 10 μL | | |
| Column Temperature | 45° C. | | |
| UV Detection | 210 nm | | |
| Run Time | 12 minutes | | |
| HPLC Column | X-Bridge C18, 4.6 × 50 mm, 3.5 μm | | |
| | Guard: X-Bridge C18, 4.6 × 20 mm, 3.5 μm | | |
| Mobile Phase A | 10 mM KH₂PO₄ in water, pH = 2.0 | | |
| Mobile Phase B | 90:10 v/v Acetonitrile:Mobile Phase A | | |
| | | % Mobile | % Mobile |
| | Time (min) | Phase A | Phase B |
| Gradient | 0 | 95 | 5 |
| | 8 | 20 | 80 |
| | 8.5 | 20 | 80 |
| | 8.51 | 95 | 5 |
| | 12 | 95 | 5 |

Data collected was wt % bupivacaine. Results are presented in the table below for drug particles of Example 1A and Example 1B. Drug particles of Example 1A are chemically stable for at least 9 months when stored at −20° C., 2-8° C., and 25° C./60% relative humidity. Drug particles of Example 1B are chemically stable for at least 9 months when stored at −20° C., 2-8° C., and 25° C.

| | | | |
|---|---|---|---|
| | | Example 1B | Example 1A |
| Time | Storage | Bupivacaine | Bupivacaine |
| Interval | Condition | Content (wt %) | Content (wt %) |

| (months) | (° C.) | AVE | STDEV | AVE | STDEV |
|---|---|---|---|---|---|
| 0 | NA | 95.70 | 1.93 | 57.13 | 2.67 |
| 1 | −20 | 88.20 | 5.64 | 56.20 | 2.22 |
| | 2-8 | 96.28 | 2.30 | 58.82 | 2.47 |
| | 25 | 88.46 | 3.79 | 58.71 | 0.89 |
| 2 | −20 | 99.16 | 0.38 | 57.03 | 0.01 |
| | 2-8 | 98.17 | 2.38 | 58.43 | 0.02 |
| | 25 | 98.92 | 2.34 | 57.52 | 2.07 |
| 3 | −20 | 96.58 | 0.46 | 57.64 | 0.66 |
| | 2-8 | 97.44 | 0.92 | 57.98 | 0.09 |
| | 25 | 97.40 | 0.34 | 57.45 | 0.33 |
| 6 | −20 | 96.79 | 0.17 | 56.86 | 0.07 |
| | 2-8 | 96.66 | 0.21 | 57.10 | 0.27 |
| | 25 | 96.55 | 0.17 | 57.35 | 0.01 |
| 9 | −20 | 96.65 | 0.14 | 57.33 | 0.22 |
| | 2-8 | 96.50 | 0.05 | 57.38 | 0.11 |
| | 25 | 96.58 | 0.02 | 57.33 | 0.08 |

Crystalline Form Via XRPD

The same two lots of drug particles were analyzed for Form I and/or Form II crystal form content of the bupivacaine free base using XRPD. The drug particles were analyzed after 3 months of storage at −20° C., 2-8° C., and 25° C./60% relative humidity. The results are shown in the table below.

| | | | |
|---|---|---|---|
| Time | Storage | Example | Example |
| Interval | Condition | 1A | 1B |

| | | |
|---|---|---|
| (months) | (° C.) | Form I (%) |

| | | | |
|---|---|---|---|
| 3 | −20 | 71.36 | 27.94 |
| | 2-8 | 69.03 | 38.53 |
| | 25 | 74.54 | 89.55 |

### Example 4

Additional lots of drug particles were manufactured and analyzed for Form I and/or Form II crystal form content of the bupivacaine free base using XRPD. The drug particles were stored at −20° C., 2-8° C., and 25° C./60% relative humidity and analyzed at various time intervals.

In one study, PLGA/bupivacaine drug particles of lot 56701120716 were manufactured according to methods disclosed herein of the present invention and bupivacaine drug particles of lot 56691100716 were manufactured according to methods disclosed herein of the present invention. In this study, Form I crystal form content was determined using XRPD at 1, 3, and 6 months for lot 56701120716 and 1 and 3 months for lot 56691100716. The results are shown in the table below.

| Time | Storage | Lot | Lot |
|---|---|---|---|
| Interval | Condition | 56701120716 | 56691100716 |

| | | |
|---|---|---|
| (months) | (° C.) | Form I (%) |

| | | | |
|---|---|---|---|
| 1 | −20 | 63.1 | 29.3 |
| | 2-8 | 68.5 | 35.3 |
| | 25 | 64.6 | 83.2 |
| 3 | −20 | 63.9 | 28.5 |
| | 2-8 | 66.1 | 42.3 |
| | 25 | 65.8 | 88.4 |
| 6 | −20 | 63.8 | NT |
| | 2-8 | 66.5 | NT |
| | 25 | 67.3 | NT |

In another study, PLGA/bupivacaine drug particles of lot 2091-001-40 were manufactured according to methods disclosed herein of the present invention and bupivacaine drug particles of lot 2091-001-36 were manufactured according to methods disclosed herein of the present invention. In this study, Form I crystal form content was determined using XRPD at 0, 1, 2, 3, 6, and 9 months for lot 2091-001-40 and 0, 1, 2, 3, and 6 months for lot 2091-001-36. The results are shown in the table below.

| Time | Storage | Lot | Lot |
|---|---|---|---|
| Interval | Condition | 2091-001-40 | 2091-001-36 |

| | | |
|---|---|---|
| (months) | (° C.) | Form I (%) |

| | | | |
|---|---|---|---|
| 0 | NA | 61.4 | 37.4 |
| 1 | −20 | 65.5 | 37.4 |
| | 2-8 | 64.4 | 47.7 |
| | 25 | 59.2 | 81.2 |
| 2 | −20 | 63.4 | 39.6 |
| | 2-8 | 61.5 | 48.1 |
| | 25 | 64.7 | 86.3 |
| 3 | −20 | 67.2 | 38.4 |
| | 2-8 | 59.9 | 51.8 |
| | 25 | 66.1 | 88.4 |
| 6 | −20 | 66.3 | 44.1 |
| | 2-8 | 64.9 | 56.4 |
| | 25 | 68.5 | 91.9 |
| 9 | −20 | 66.0 | NT |
| | 2-8 | 66.1 | NT |
| | 25 | NT | NT |

### Example 5

In Vivo Study to Determine the Efficacy in the Thermal Sensitivity (Hargreaves' Test) Endpoint in the Rat

Compositions of bupivacaine free base microparticles were evaluated in a thermal model (Hargreaves Test) of pain in the rat following perineural (sciatic nerve) infiltration. Compositions were administered to Sprague Dawley rats with N=11-12/group.

The four test articles studied included:

(1) PLGA/bupivacaine particles 926-144-3, manufactured in accordance with Example 1A (particles were stored 20 days at 40° C./25% relative humidity prior to harvesting),

(2) Bupivacaine particles 926-144-1 manufactured in accordance with Example 1B,

(3) Vehicle, 969-37-1, and

(4) Exparel (bupivacaine liposome injectable suspension) (13.3 mg/mL, Pacira Pharmaceuticals, Inc., San Diego, Calif.), a comparative control.

An aqueous vehicle, 969-37-1, was manufactured. The aqueous vehicle contained 0.5 wt % sodium hyaluronate (HA, 1,000 kDa, Stanford, catalog HA-EP-1.8), 0.1 wt % polysorbate 80 (NOF, catalog HX2), and 200 mM Tris (as Tris base and Tris HCl, JT Baker, catalog 4109-01 and 4106-01, respectively), pH 8. The vehicle was manufactured by combining 0.147 g HA, 0.028 g PS80, 0.291 g Tris base, 0.563 g Tris HCl, and 28.983 g WFI in a 50 mL sterile conical tube. The tube was rotated end to end at room temperature until all components were in solution. Prior to injection, the particles were suspended in vehicle. Vehicle was added to the particles in the vial and vortexed for at least two minutes until a homogenous suspension was present. If a homogeneous suspension was not present after two minutes of vortexing; the vial was vortexed until visual homogeneity was achieved. The bupivacaine concentration of the suspension was approximately 33.3 mg/mL.

Both 926-144-3 and 926-144-1 (in suspension) were dosed at 1.2 mL/kg, delivering 40 mg/kg bupivacaine. The vehicle only control was dosed at 1.2 mL/kg. Exparel (bupivacaine liposome injectable suspension) (Pacira Pharmaceuticals, Inc., San Diego, Calif.), was dosed at 1.4 mL/kg, delivering 18.6 mg/kg bupivacaine.

The sciatic nerve of the rat was exposed under anesthesia; and the test article was injected directly onto the sciatic nerve. The muscle was sutured closed, and the skin was closed using tissue adhesive.

Paw withdrawal after applying radiant heat was assessed in the rats prior to dosing and at 2, 4, 5.5, and 7 hours following perineural administration. Baseline and post-dosing thermal sensitivity was measured using a radiant heat plantar test apparatus.

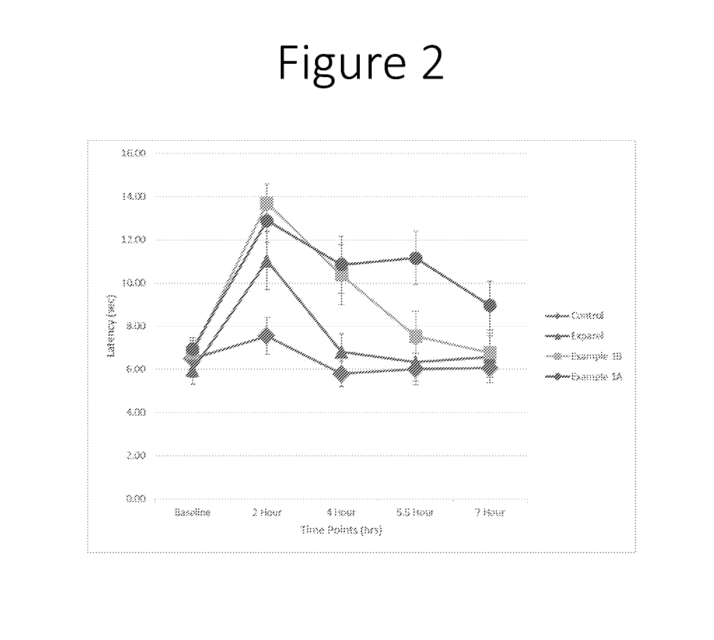

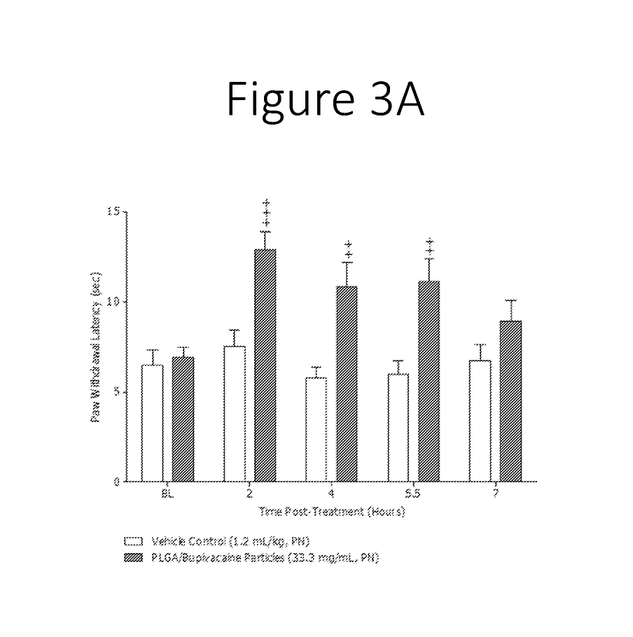

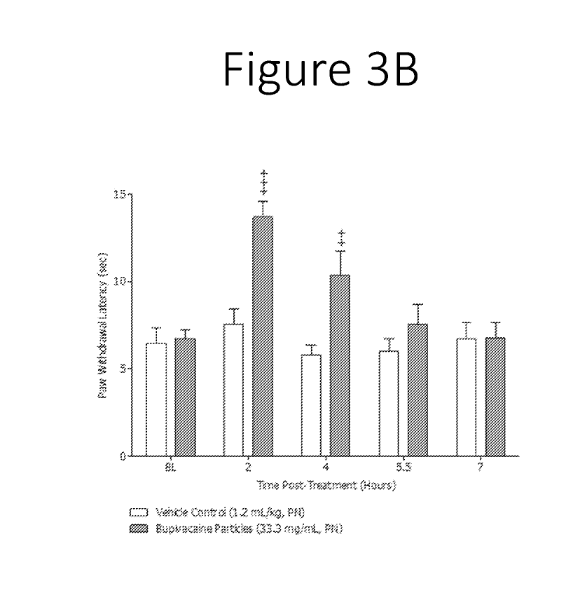

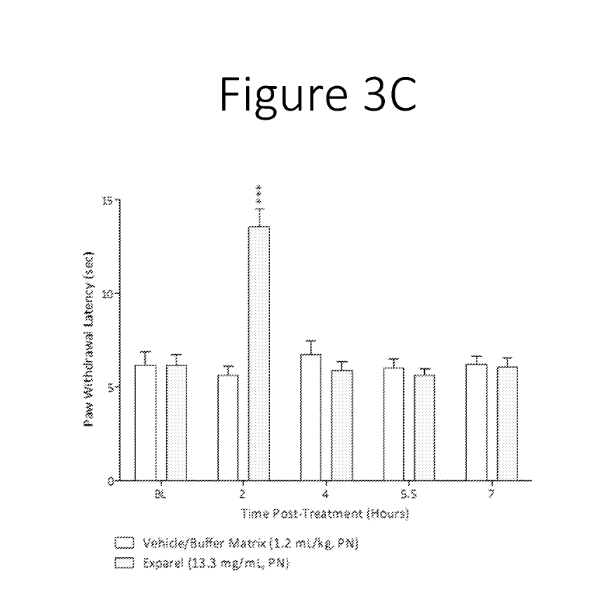

As shown in FIGS. 3A, 3B, and 3C, perineural administration of PLGA/bupivacaine particles significantly increased paw withdrawal latencies at 2, 4, and 5.5 hours after dosing compared to vehicle only control animals. Perineural administration of bupivacaine particles significantly increased paw withdrawal latencies at 2 and 4 hours after dosing, while perineural administration of Exparel (bupivacaine liposome injectable suspension), liposomal bupivacaine, significantly increased paw withdrawal latencies for 2 hours after dosing. FIG. 2 depicts the data as a scatter plot. FIGS. 3A, 3B, and 3C depict the data as bar graphs. As shown in FIG. 3A, the PLGA/bupivacaine particles (33.3 mg/mL, 40 mg/kg), by perineural administration was significantly different compared to the vehicle control at 2, 4, and 5.5 hours (++/+++: p<0.01/0.001 unpaired t-test). As shown in FIG. 3B, the bupivacaine particles (33.3 mg/mL, 40 mg/kg), by perineural administration was significantly different compared to the vehicle control at 2 and 4 hours (++/+++: p<0.01/0.001 unpaired t-test). As shown in FIG. 3C, the Exparel (bupivacaine liposome injectable suspension) (13.3 mg/mL, 18.6 mg/kg), by perineural administration was significantly different compared to the vehicle control at 2 hours only (***: p<0.001 Dunnett's post hoc test versus baseline (BL)).

PLGA/bupivacaine particles significantly increased paw withdrawal latencies for at least 5.5 hours after dosing. Bupivacaine particles significantly increased paw withdrawal latencies for at least 4 hours after dosing. Exparel (bupivacaine liposome injectable suspension), liposomal bupivacaine, significantly increased paw withdrawal latencies. Bupivacaine delivered in particle form provided significantly increased paw withdrawal latencies compared to bupivacaine delivered in liposomal form.

### Example 6

A Non-GLP Pharmacokinetic Evaluation in the Rat

A study was conducted to evaluate the impact of pH and vehicle viscosity on pharmacokinetic parameters of bupivacaine when administered as particles of the invention.

Two buffers and two vehicles were prepared for use in the study.

Buffer 1, 969-08-1: This aqueous buffer contained 90 mM sodium chloride, 27 mM sodium acetate, 23 mM sodium gluconate, 5 mM potassium chloride, 1 mM magnesium chloride, and 0.1 wt % polysorbate 80. The buffer was filtered through a 0.2 μm PES filter. The pH was approximately 7.37.

Buffer 2, 969-08-2: This aqueous buffer contained 476 mM sodium bicarbonate, 7 mM EDTA, and 0.1 wt % PS80. The buffer was filtered through a 0.2 μm PES filter. The pH was approximately 8.

Vehicle 1, 969-07-1: This aqueous vehicle contained 1 wt % hyaluronic acid (1,000 kDa, Creative PEGworks, catalog HA-105), 145 mM sodium chloride, and 1.6 mM sodium phosphate (incorporated as dibasic sodium phosphate, anhydrous and monobasic sodium phosphate, monohydrate). The pH was approximately 7.41.

Vehicle 2 (969-07-2): This aqueous vehicle contained 1 wt % hyaluronic acid (2.500 kDa, Creative PEGworks, catalog HA-107), 145 mM sodium chloride, 1.9 mM sodium phosphate (incorporated as dibasic sodium phosphate, anhydrous and monobasic sodium phosphate, monohydrate). The pH was approximately 7.41.

Two particle compositions were tested in this study: PLGA/bupivacaine particles and bupivacaine particles.

PLGA/bupivacaine particles, 926-132-1 were manufactured in accordance with Example 1A with the following changes. A 22 wt % solids homogeneous solution of 50 wt % PLGA (Evonik Industries, Resomer RG502H) and 50 wt % bupivacaine free base (Cayman Chemical Company) in ethyl acetate was prepared. The mold/film was passed through a laminator at 300° F. at 5 feet/minute. Particles were stored while in the mold at 40° C./25% relative humidity for approximately 13 days prior to harvesting.

Bupivacaine particles, 926-135-1, were manufactured in accordance with Example 1B with the following changes. A 40 wt % solids homogeneous solution of 100 wt % bupivacaine free base (Cayman Chemical Company) in chloroform was used. Particles were stored while in the mold at ambient conditions for approximately 4 days prior to harvesting.

Prior to injection, the particles were suspended in vehicle. Vehicle was added to the particles in the vial and vortexed for at least two minutes until a homogenous suspension was present. If a homogeneous suspension was not present after two minutes of vortexing; the vial was vortexed until visual homogeneity was achieved. The bupivacaine concentration of the suspension was approximately 33.3 mg/mL.

The table below details the buffer and vehicle additions.

| | | | | Vehicle | |
|---|---|---|---|---|---|
| Animal | | Buffer | | Vehicle | |
| Group | Particle ID | Buffer ID | Volume, mL | ID | Volume, mL |
| 1 | 926-135-1 | 969-08-2 | 2.058 | 969-07-1 | 2.208 |
| 2 | 926-135-1 | 969-08-1 | 2.058 | 969-07-01 | 2.208 |
| 3 | 926-135-1 | 969-08-2 | 2.941 | 969-07-2 | 1.325 |
| 4 | 926-135-1 | 969-08-1 | 2.941 | 969-07-2 | 1.325 |
| 5 | 926-132-1 | 969-08-2 | 1.886 | 969-07-1 | 2.211 |
| 6 | 926-132-1 | 969-08-1 | 1.886 | 969-07-01 | 2.211 |
| 7 | 926-132-1 | 969-08-2 | 2.770 | 969-07-2 | 1.326 |
| 8 | 926-132-1 | 969-08-1 | 2.770 | 969-07-2 | 1.326 |

Sprague Dawley rats (3/group) were administered a single SC dose of 926-132-1 or 926-135-1 in buffer/vehicle suspension at 1.2 mL/kg (33.3 mg/mL bupivacaine) for a total bupivacaine dose of 40 mg/kg.

The pH was measured using an Oakton pH meter before and after dosing. The table below summarizes the data.

Blood samples for PK analysis were collected from each animal prior to dosing, and at 15 and 30 minutes, 1, 2, 4, 8, 24, 48, and 72 hours after dosing. Plasma concentrations of bupivacaine were measured by LC-MS/MS and PK parameters were calculated using PK Functions for Microsoft Excel.

PK results for each animal group are summarized in the tables below.

| | | | | Elimination | | AUC |
|---|---|---|---|---|---|---|
| Animal | | Cₘₐₓ | Tₘₐₓ | Rate | Half-life | (0-72 |
| Group | N | (ng/mL) | (hours) | Constant | (hours) | hour) |
| 1 | 3 | 462.7 | 8.00 | 0.0524 | 14.49 | 12611.76 |
| 2 | 3 | 347.7 | 5.33 | 0.0527 | 14.19 | 10528.24 |
| 3 | 3 | 350.7 | 12.00 | 0.0450 | 16.17 | 10434.08 |
| 4 | 3 | 366.0 | 12.00 | 0.0673 | 10.79 | 10766.93 |
| 5 | 3 | 329.3 | 5.30 | 0.0381 | 18.26 | 10322.13 |
| 6 | 3 | 524.7 | 2.80 | 0.0462 | 15.35 | 10697.06 |
| 7 | 3 | 308.3 | 6.70 | 0.0366 | 18.98 | 9412.60 |
| 8 | 3 | 690.7 | 1.8 | 0.0423 | 16.85 | 9599.79 |

The coefficient of variation is shown in the table below.

| | | | | Elimination | | AUC |
|---|---|---|---|---|---|---|
| Animal | | Cₘₐₓ | Tₘₐₓ | Rate | Half-life | (0-72 |
| Group | N | (ng/mL) | (hours) | Constant | (hours) | hour) |
| 1 | 3 | 40.8% | 0.0% | 38.9% | 34.2% | 13.3% |
| 2 | 3 | 10.8% | 43.3% | 31.3% | 35.5% | 6.2% |
| 3 | 3 | 41.9% | 88.2% | 25.9% | 27.5% | 19.8% |
| 4 | 3 | 25.1% | 88.2% | 24.5% | 27.7% | 8.8% |
| 5 | 3 | 29.9% | 43.3% | 7.8% | 7.5% | 15.5% |
| 6 | 3 | 50.6% | 71.3% | 19.3% | 17.9% | 26.7% |
| 7 | 3 | 7.5% | 34.6% | 6.1% | 6.2% | 8.3% |
| 8 | 3 | 56.6% | 103.3% | 19.5% | 21.9% | 8.0% |

The PK parameters were similar for Groups 1 through 4 with some slight variation for Tₘₐₓ. The Cₘₐₓ values ranged from 348 to 366 ng/mL and AUCₗₐₛₜ ranged from 10434 to 12612 ng·hr/mL. The mean Tₘₐₓ ranged from 5.33 hours for Groups 2 to 8 hours for bupivacaine particles; Tₘₐₓ was 12 hours for both Groups 3 and 4. The elimination rate constant ranged from 0.045 to 0.064 and mean t_{1/2} from 10.8 to 16.2 hours. Thus, differences in pH or HA vehicle had little effect on bupivacaine PK of 926-135-1.

The PK parameters were similar for Group 5 through 8 for AUCₗₐₛₜ, elimination rate constant, and t_{1/2}, but with some differences for Cₘₐₓ and Tₘₐₓ. The formulations with the shortest duration to Tₘₐₓ, Groups 6 and 8, also exhibited the higher Cₘₐₓ values. The mean Tₘₐₓ for Groups 2 and 4 were 2.8 and 1.8 hours, respectively, compared to 5.3 and 6.7 hours for Groups 5 and 7, respectively. The mean AUCₗₐₛₜ values ranged from 9412 ng·hr/mL to 10697 ng·hr/mL. The elimination rate constant ranged from 0.037 to 0.046 and mean t_{1/2} from 15.4 to 19.0 hours. The release characteristics of 926-132-1 appeared to be altered by the vehicle pH, but not by the type of HA used in the vehicle.

### Example 7

Pharmacokinetic Analysis Studies

Studies were conducted to assess the PK of bupivacaine following a single SC injection of compositions of the invention.

7.1: Bupivacaine Particles

Bupivacaine particles, 976-27-2, were manufactured in accordance with Example 1B with the following changes. Particles were stored in the mold at ambient conditions for approximately 12 days prior to harvesting.

A vehicle, 1019-49, was also manufactured. First, a stock composition containing 1.0 wt % hyaluronic acid (1,000 kDa, Stanford, catalog HA-EP-1.8), 50 mM Tris (as Tris base and Tris HCl), 100 mM sodium chloride, 0.1 wt % polysorbate 80 was made. The viscosity of the stock composition was approximately 2000 to 3500 cps. Second, a diluting composition containing 50 mM Tris (as Tris base and Tris HCl), 100 mM sodium chloride, 0.1 wt % polysorbate 80 was made. The pH of the diluting composition was adjusted to approximately 8. The diluting composition was sterile filtered. The stock composition was diluted with the diluting composition by combining 75 wt % stock composition and 25 wt % diluting composition. The final composition of the vehicle was 0.75 wt % hyaluronic acid (1,000 kDa, Stanford, catalog HA-EP-1.8), 50 mM Tris (as Tris base and Tris HCl), 100 mM sodium chloride, 0.1 wt % polysorbate 80. The pH was approximately 8 and the viscosity was approximately 838 cps.

Prior to injection, the particles were suspended in a vehicle. Vehicle was added to achieve the dosing concentrations in the table below. To form the suspension, vehicle was added to a vial containing the particles. The vial was vortexed for approximately 30 seconds. After vortexing, the vial was sonicated for approximately 30 seconds. The vial was vortexed/sonicated for 3 cycles until a uniform suspension was formed. After aspirating the desired volume into a syringe, the suspension was further mixed by syringe to syringe mixing through a female-female luer lock connector.

In this study, Sprague Dawley rats (N=6/sex/group, except 80 mg/kg where N=6 males) were administered 976-27-2 in suspension at 40, 80, 120, and 160 mg/kg (26.7, 53.3, 80.0, and 106.7 mg/mL bupivacaine respectively) or Exparel (bupivacaine liposome injectable suspension) (Pacira Pharmaceuticals, Inc., San Diego, Calif.) at 40 mg/kg (13.3 mg/mL).

| | Bupivacaine | | |
|---|---|---|---|
| | Concentration, | | Bupivacaine Dose, |
| Animal Group | mg/mL | Dose, mL/kg | mg/kg |
| 1-Exparel | 13.3 | 3.0 | 40 |
| 2 | 26.7 | 1.5 | 40 |
| 3 | 53.3 | 1.5 | 80 |
| 4 | 80.0 | 1.5 | 120 |
| 5 | 106.7 | 1.5 | 160 |

Blood samples for PK analysis were collected at 0.25, 0.75, 1.5, 2, 4, 6, 8, 24, 30, 48, 72 and 96 hours after dosing. Plasma concentrations of bupivacaine were measured using LC-MS/MS with a range from 0.500 to 500 ng/mL. PK analysis was conducted using WinNonlin (noncompartmental analysis). PK parameters are summarized in the table below.

| | Exparel, mg/kg | | 976-27-2 in suspension, mg/kg | | | | | | |
|---|---|---|---|---|---|---|---|---|---|
| PK | 40 | | 40 | | 80 | 120 | | 160 | |
| Parameter | Male | Female | Male | Female | Male | Male | Female | Male | Female |
| Tₘₐₓ (hr) | 1.5 | 1.5 | 1.5 | 4 | 2 | 4 | 2 | 4 | 2 |
| t_{1/2} (hr) | 16.5 | 14.0 | 7.6 | 4.2 | 11.9 | 28.8 | 22.6 | 20.2 | 19.5 |
| Cₘₐₓ | 720 ± | 1120 ± | 860 ± | 850 ± | 850 ± | 640 ± | 1520 ± | 740 ± | 1980 ± |
| (ng/mL) | 190 | 160 | 230 | 150 | 160 | 380 | 1100 | 150 | 1290 |
| AUCₗₐₛₜ | 7170 ± | 7970 ± | 10590 ± | 10250 ± | 15260 ± | 14210 ± | 10670 ± | 20030 ± | 24160 ± |
| (ng · hr/mL) | 450 | 750 | 1200 | 940 | 960 | 3470 | 4440 | 4230 | 1950 |

Exposure to bupivacaine following 976-27-2 administration, based on both Cₘₐₓ and AUCₗₐₛₜ, did not consistently increase with increasing dose, with the exception of Cₘₐₓ for females.

The increase in Cₘₐₓ in females was less than dose proportional from 40 to 160 mg/kg, with a 4-fold increase in dose resulting in a 2.3-fold increase in exposure. However, one female in each dose group had a high Cₘₐₓ value (>3600 ng/mL for 120 mg/kg female and >4500 ng/mL for 160 mg/kg female), which contributed to the higher mean Cₘₐₓ values for females in these two dose groups. In males, the Cₘₐₓ was similar across all 4 doses.

The increase in AUCₗₐₛₜ across the dose range for females was less than dose proportional, characterized by no increase in AUCₗₐₛₜ from 40 to 120 mg/kg and a 2.3-fold increase for a 1.3-fold increase in dose from 120 to 180 mg/kg. Similarly in males, the increase in AUCₗₐₛₜ across the dose range was less than dose proportional. The increase in exposure associated with a 2-fold increase in dose from 40 to 80 mg/kg was 1.4-fold, but no increase in exposure was noted from 80 to 120 mg/kg. The increase in AUCₗₐₛₜ exposure from 120 to 160 mg/kg was dose proportional (1.3-fold increase in dose resulting in a 1.4-fold increase in exposure). No consistent gender related differences (i.e., ≤2-fold difference in any parameter) were identified.

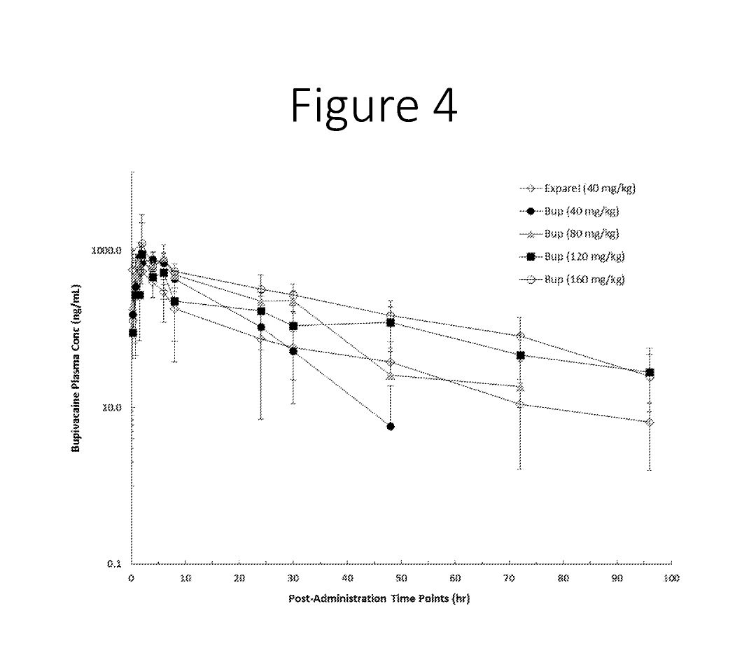

The mean bupivacaine Tₘₐₓ for 976-27-2 ranged from 1.5 to 4 hours and the t_{1/2} increased from the 40 to 120 mg/kg ranging from 4.2 to 22.6 hours for females and 7.6 to 28.8 hours for males. No additional increase in t_{1/2} was observed at 160 mg/kg (19.5 and 20.2 hours in females and males, respectively). Plasma concentrations of bupivacaine were noted generally (at least 2-3/sex) through 30 hours at the 40 mg/kg dose of 976-27-2 (mean M/F combined at 30 hr=55 ng/mL). At doses of 80 to 160 mg/kg, plasma concentrations were observed through 72 to 96 hours (mean M/F combined values at 72-96 hours ranging from 20-90 ng/mL). Plasma bupivacaine concentrations are shown in FIG. 4.

In conclusion, the lack of increasing Cₘₐₓ with increasing dose in males and a generally less than dose-proportional increase in females, the prolonged time to reach peak plasma levels and the increasing t_{1/2} with increasing dose demonstrate release of bupivacaine to systemic circulation without an initial burst release following subcutaneous administration of 976-27-2 particles.

7.2. Bupivacaine/PLGA Particles

Particles, 914-95-2, were manufactured in accordance with Example 1A with the following changes. A 28 wt % solids homogeneous solution of 40 wt % PLGA (Evonik Industries, Resomer RG502H) and 60 wt % bupivacaine free base (Cayman Chemical Company) in acetone was prepared. The mold/film was passed through a laminator at 290° F. at 10 feet/minute. Particles were stored while in the mold at ambient conditions for approximately 19 days prior to harvesting.

The vehicle used in this study was 1019-49, which is described above.

Prior to injection, the particles were suspended in a vehicle. Vehicle was added to achieve the dosing concentrations in the table below. To form the suspension, vehicle was added to a vial containing the particles. The vial was vortexed for approximately 30 seconds. After vortexing, the vial was sonicated for approximately 30 seconds. The vial was vortexed/sonicated for 3 cycles until a uniform suspension was formed. After aspirating the desired volume into a syringe, the suspension was further mixed by syringe to syringe mixing through a female-female luer lock connector.

In this study, Sprague Dawley rats (N=6/sex/group, except for soluble bupivacaine group where N=6 males) were administered 914-95-2 in suspension at 40, 80, and 169.2 mg/kg bupivacaine concentration (26.7, 53.3, and 80.0 mg/mL bupivacaine) or bupivacaine hydrochloride solution (Marcaine, 0.75%), at 10 mg/kg (7.5 mg/mL).

| | Bupivacaine | | Bupivacaine |
|---|---|---|---|
| Animal Group | Concentration, mg/mL | Dose, mL/kg | Dose, mg/kg |
| 1-Marcaine | 7.5 | 1.35 | 10 |
| 2 | 26.7 | 1.5 | 40 |
| 3 | 53.3 | 1.5 | 80 |
| 4 | 112.8 | 1.5 | 169.2 |

Blood samples (N=3/sex/group/time point) for PK analysis were collected at 0.25, 0.75, 1.5, 2, 4, 6, 8, 24, 30, 48, 72 and 96 hours after dosing. Plasma concentrations of bupivacaine were measured using LC-MS/MS with a range from 0.500 to 500 ng/mL. PK analysis was conducted using WinNonlin (noncompartmental analysis). PK parameters are summarized in the table below.

| | Marcaine | | | | | | |
|---|---|---|---|---|---|---|---|
| | (mg/kg) | 914-95-2, in suspension (mg/kg) | | | | | |
| | 10 | 40 | | 80 | | 169.2 | |
| | Male | Male | Female | Male | Female | Male | Female |
| PK Parameter | (N = 6) | (N = 6) | (N = 6) | (N = 6) | (N = 6) | (N = 6) | (N = 6) |
| Tₘₐₓ (hr) | 0.3 | 4 | 4 | 4 | 4 | 1.5 | 1.5 |
| t_{1/2} (hr) | 1 | 8.6 | 6.6 | 14 | 18.3 | 31.6 | 24.5 |
| Cₘₐₓ (ng/mL) | 1004 ± 31.9 | 689 ± 72.7 | 865 ± 221 | 786 ± 373 | 701 ± 130 | 776 ± 55.3 | 911 ± 215 |
| AUCₗₐₛₜ | 1856 ± 62.4 | 7645 ± 625 | 10547 ± 1126 | 12264 ± 452 | 14630 ± 1145 | 19735 ± 1419 | 23219 ± 914 |
| (ng · hr/mL) | | | | | | | |

There was no consistent gender-related difference in any of the assessed pharmacokinetic parameters (i.e., ≤2-fold difference in any parameter). Following administration of 914-95-2, plasma bupivacaine AUC increased with increasing dose. However, mean Cₘₐₓ values were similar across the dose groups: Cₘₐₓ=777, 744, and 844 ng/mL for 40, 80, and 169.2 mg/kg respectively, male and female results averaged. Across the dose range of 40 to 169.2 mg/kg, the increase in exposure was less than dose proportional for Cₘₐₓ and AUC, but with a roughly dose proportional increase in AUC from 40 to 80 mg/kg.

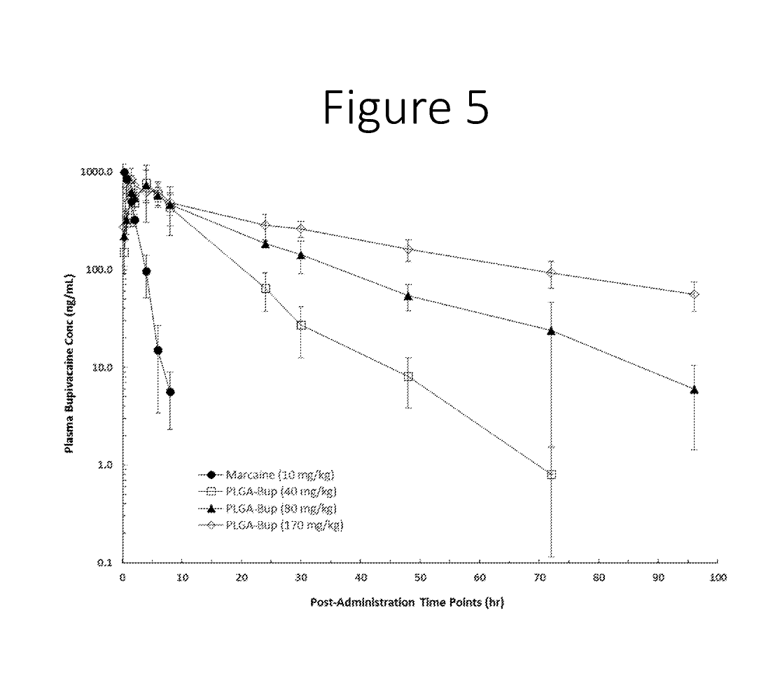

The kinetic profile of 914-95-2 was notably different from that of bupivacaine hydrochloride solution as evidenced by a Tₘₐₓ of generally 4 hours (1.5 hours at 169.2 mg/kg) vs. 18 minutes, respectively. Plasma levels of bupivacaine could be detected through 8 hours (mean at 8 h=5.61 ng/mL) for the bupivacaine hydrochloride solution, through 72 hours for 40 mg/kg 914-95-2 (mean M/F combined at 72 h=0.805 ng/mL), and through 96 hours for 80 and 169.2 mg/kg 914-95-2 (mean M/F combined at 96 h=5.94 and 56.1 ng/mL, respectively). The resulting mean t_{1/2} was 1 hour for bupivacaine solution, but the mean t_{1/2} for 914-95-2 increased dose dependently and ranged from 6.6 to 31.6 hours. While the bupivacaine doses for 914-95-2 were approximately 5- to 23-fold higher than that administered for the bupivacaine solution, the Cₘₐₓ values for 914-95-2 were only 0.7- to 0.9-fold the Cₘₐₓ for the bupivacaine solution. The AUCₗₐₛₜ values for 914-27-2, however, were approximately 5- to 12-fold higher than for the bupivacaine solution. Plasma bupivacaine concentration-time curves are shown in FIG. 5.

In conclusion, the lack of increasing Cₘₐₓ with increasing dose, the prolonged time to reach peak plasma levels and the increasing t_{1/2} with increasing dose demonstrates the release from the bupivacaine to systemic circulation without an initial burst release following subcutaneous administration of 914-27-2 particles.

### Example 8

Subcutaneous Toxicity Studies: Toxicokinetic Analysis and Histological Studies

Studies were conducted to determine the systemic exposure to bupivacaine following a single subcutaneous administration of compositions of the invention.

Blood samples from a cohort of male and female Sprague Dawley rats were analyzed to assess the systemic exposure to bupivacaine following a single subcutaneous administration of compositions of the invention. Blood samples were taken at 11 time points: 0.5, 1.25, 2.5, 4, 6, 8, 24, 30, 48, 72 and 96 hours following administration. Plasma concentrations of bupivacaine were measured using LC-MS/MS and PK analysis was conducted using WinNonlin version 6.3.

8.1. Bupivacaine Particles

Bupivacaine particles, 1031-13, were manufactured in accordance with Example 1B with the following changes. The mold/film was passed through a laminator at 300° F. at 10 feet/minute. Particles were stored in the mold at ambient conditions for approximately 14 days prior to harvesting.

A vehicle, 1072-7, was manufactured. First, a stock composition containing 1.0 wt % hyaluronic acid (1,000 kDa, Stanford, catalog HA-EP-1.8), 50 mM Tris (as Tris base and Tris HCl), 100 mM sodium chloride, 0.1 wt % polysorbate 80 was made. The stock composition was autoclaved. The viscosity of the stock composition was approximately 2000 to 3500 cps. Second, a diluting composition containing 50 mM Tris (as Tris base and Tris HCl), 100 mM sodium chloride, 0.1 wt % polysorbate 80 was made. The pH of the diluting composition was adjusted to approximately 8. The diluting composition was sterile filtered. The stock composition was diluted with the diluting composition. The vehicle had a final composition of 0.61 wt % hyaluronic acid (1,000 kDa, Stanford, catalog HA-EP-1.8), 50 mM Tris (as Tris base and Tris HCl), 100 mM sodium chloride, 0.1 wt % polysorbate 80. The pH was approximately 8.02. The viscosity was approximately 382 cps.

Prior to injection, the particles were suspended in vehicle. Vehicle was added to achieve the dosing concentrations in the table below. To form the suspension, vehicle was added to a vial containing the particles. The vial was vortexed for approximately 30 seconds. After vortexing, the vial was sonicated for approximately 30 seconds. The vial was vortexed/sonicated for a minimum of five cycles until a uniform suspension was formed. After aspirating the desired volume into a syringe, the suspension was further mixed by syringe to syringe mixing through a female-female luer lock connector. The table below details bupivacaine concentration of the suspensions and the bupivacaine doses.

| | Bupivacaine | | |
|---|---|---|---|
| | Concentration, | | Bupivacaine Dose, |
| Animal Group | mg/mL | Dose, mL/kg | mg/kg |
| 1-Control/Vehicle | 0 | 1.2 | 0 |
| 2 | 33 | 1.2 | 40 |
| 3 | 67 | 1.2 | 80 |
| 4 | 133 | 1.2 | 160 |

1031-13 was dosed at dose levels of 40, 80, and 160 mg/kg. PK parameters were analyzed and are summarized in the table below. Note, for both males and females at the 160 mg/kg dose, the terminal rate constant could not be adequately estimated.

| | 1031-13, in suspension (mg/kg) | | | | | |
|---|---|---|---|---|---|---|
| | 40 | | 80 | | 160 | |
| | Male | Female | Male | Female | Male | Female |
| PK Parameter | N = 6 | N = 6 | N = 6 | N = 6 | N = 6 | N = 6 |
| Tₘₐₓ (hr) | 4 | 4 | 6 | 4 | 6 | 6 |
| t_{1/2} (hr) | 9.8 | 11.5 | 31.6 | 31.4 | — | — |
| Cₘₐₓ (ng/mL) | 282 | 240 | 263 | 289 | 405 | 302 |
| AUCₗₐₛₜ | 7140 | 5940 | 10800 | 11200 | 18700 | 15600 |
| (ng · hr/mL) | | | | | | |

Tₘₐₓ was 4 or 6 hours following administration indicating that subcutaneously administered 1031-13 did not release all of the drug immediately (“dose dump”). Tₘₐₓ tended to be later at the higher dose levels indicating that an increase in the subcutaneous dose tended to prolong the absorption phase.

The systemic exposure, as measured by Cₘₐₓ and AUCₗₐₛₜ, of bupivacaine increased with increasing dose over the dose range of 40 to 160 mg/kg. However, these increases were less than dose proportional. Overall, the Cₘₐₓ and AUCₗₐₛₜ values at the highest dose level of 160 mg/kg were approximately 66% and 34% lower, respectively, than values predicted from a linear relationship. Exposure was generally similar between males and females.

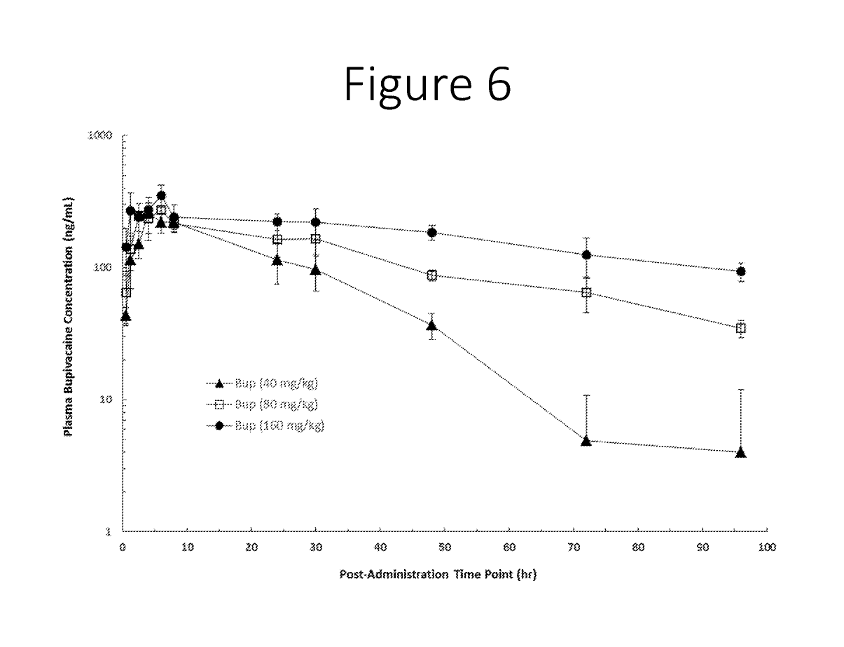

The terminal half-life could not be estimated adequately for all test groups, but where estimated, it appeared to be independent of sex and increased with increasing dose. The mean t_{1/2} increased approximately 3-fold with a 2-fold increase in dose; t_{1/2} was ˜10.7 and 31.5 hours for the 40 and 80 mg/kg dose levels (genders combined), respectively. Plasma bupivacaine concentration-time curves are shown in FIG. 6.

The rate and extent of systemic exposure of rats to bupivacaine appeared to be characterized by nonlinear (dose-dependent) kinetics following a single subcutaneous administration of 1031-13 over the dose range of 40 to 160 mg/kg. Increasing the dose of 1031-13 above 40 mg/kg is likely to result in a lower systemic exposure than would be predicted from a linear relationship, which is consistent with dissolution-rate limited absorption of bupivacaine. As such, the Cmax does not increase at an equivalent rate as AUC suggesting that 1031-13 is able to provide a sustained exposure with a potentially improved tolerability profile.

The toxicity of a single dose administered subcutaneously was also evaluated histologically in rats.

Sprague-Dawley rats (N=15/sex/group main study; N=3/sex/control and 6/sex/test article group for TK cohort) were administered a single subcutaneous dose of 1031-13 at 40, 80, and 160 mg/kg, or vehicle control in a study designed to evaluate the local toxicity and TK of the test article. In the main study groups, 10/sex/group were euthanized on Day 3 and 5/sex/group were euthanized on Day 15.

There were no adverse test article-related effects on clinical observations (excluding dermal observations), body weight or body weight change, food consumption, hematology, coagulation, serum chemistry, urinalysis, organ weights, or gross necropsy.

On Day 3, changes were present in the subcutis in all males and females dosed at ≥40 mg/kg. A single well circumscribed pale area was present in the fibrous connective tissue component of the subcutis immediately subjacent to the panniculus muscle. The pale area consisted of a large population of variably sized particles consistent with the test item. At the periphery of the pale area, the particles diminished in size and their borders collapsed together to form an eosinophilic layer that interfaced with an inflammatory response in the surrounding tissue. The inflammatory response included neutrophilic inflammatory cell infiltrates, congestion/vascular dilation and necrosis. The inflammatory response was similar in incidence and severity across dose groups.

Changes observed at both vehicle control and 1031-13 injection sites, included mononuclear inflammatory cell infiltrates (composed of macrophages, fewer lymphocytes and occasional plasma cells along with immature fibroblasts) and edema. These changes were greater in severity in the 1031-13 groups (at all dose levels) than the vehicle controls. Within the 1031-13 groups, these changes exhibited a dose-related increase in severity in females but were similar across dose groups for males. Inflammatory cell infiltrates at the marked and severe level often involved the surrounding muscle.

On Day 15, pale areas, necrosis and neutrophilic infiltrates persisted in one or both sexes at 160 mg/kg and in 1 male (pale areas only) at 80 mg/kg. All changes were decreased in incidence compared to corresponding dose group at Day 3. Pale areas and necrosis were also decreased in severity. Fatty infiltrates were present in both sexes at ≥80 mg/kg and 1 female at 40 mg/kg exhibited a dose-related increase in incidence and severity. All changes were located in the fibrous connective tissue component of the subcutis immediately subjacent to the panniculus muscle.

Mononuclear inflammatory cell infiltrates were present at all dose levels (including controls) but were decreased in severity compared to corresponding dose groups at Day 3. The severity of the infiltrates was greater at the 1031-13 injection sites than in the control sites (minimal severity) at all dose levels, particularly at 80 and 160 mg/kg, and exhibited a dose dependent relationship. Infiltrates in the control sites were composed primarily of lymphocytes with rare macrophages and were frequently oriented around blood vessels. Infiltrates in the 1031-13 sites were composed of admixtures of lymphocytes, variable numbers of macrophages and rare giant cells. Macrophages at the 1031-13 injection sites had vacuolated cytoplasm. Slight to minimal fibrosis occurred in one or both sexes in all dose groups but was increased in incidence and severity in males at 160 mg/kg. Neovascularization was observed in both sexes primarily at the 160 mg/kg 1031-13 dose level.

8.2. PLGA/Bupivacaine Particles

Particles, 1031-12, were manufactured in accordance with Example 1A with the following changes. A 28 wt % solids homogeneous solution of 40 wt % PLGA (Evonik Industries, Resomer RG502H) and 60 wt % bupivacaine free base (Cayman Chemical Company) in acetone was prepared. The mold/film was passed through a laminator at 290° F. at 10 feet/minute. Particles were stored while in the mold at approximately 40° C./25% relative humidity for approximately 11 days prior to harvesting.

In addition, placebo particles, 914-95-4, were also manufactured. First, a particle stock solution was prepared. A 28 wt % solids homogeneous solution of 100 wt % PLGA (Evonik Industries, Resomer RG502H) in acetone was prepared. The particle stock solution was filtered through a sterile 0.2 μm PTFE filter. The resulting particle stock solution was cast at room temperature onto a 4 mil PET film pre-coated with a polyvinyl alcohol harvest layer. The dried cast film was laminated against a 25 μm hexagon mold (Liquidia Technologies, Inc. Morrisville, N.C.). The mold/film was passed through a laminator at 290° F. at 10 feet/minute. Particles were harvested and sterilized.

The vehicle used in this study was 1072-7, which is described above.

Prior to injection, the particles were suspended in vehicle. Vehicle was added to achieve the dosing concentrations in the table below. To form the suspension, vehicle was added to a vial containing the particles. The vial was vortexed for approximately 30 seconds. After vortexing, the vial was sonicated for approximately 30 seconds. The vial was vortexed/sonicated for a minimum of three cycles until a uniform suspension was formed. After aspirating the desired volume into a syringe, the suspension was further mixed by syringe to syringe mixing through a female-female luer lock connector. The table below details bupivacaine concentration of the suspensions and the bupivacaine doses.

| | Bupivacaine | | |
|---|---|---|---|
| | Concentration, | | Bupivacaine Dose, |
| Animal Group | mg/mL | Dose, mL/kg | mg/kg |
| 1-Control/Vehicle | 0 | 1.2 | 0 |
| 2-Control/Placebo | 0 | 1.2 | 0 |
| 3 | 16.7 | 1.2 | 20 |
| 4 | 67 | 1.2 | 80 |

1031-12 was dosed at dose levels of 20 or 80 mg/kg. PK parameters are summarized in the table below. Note, for males at the 20 mg/kg dose, the terminal rate constant could not be adequately estimated.

| 1031-12, in suspension | |
|---|---|
| (mg/kg) | |
| 20 | 80 |

| PK Parameter | Male | Female | Male | Female |
|---|---|---|---|---|
| Tₘₐₓ (hr) | 6 | 6 | 1.25 | 6 |
| t_{1/2} (hr) | — | 6.6 | 30.8 | 24.1 |
| Cₘₐₓ (ng/mL) | 148 | 187 | 322 | 243 |
| AUCₗₐₛₜ | 2420 | 3020 | 8760 | 7320 |
| (ng · hr/mL) | | | | |

Tₘₐₓ was 6 hours except for the 80 mg/kg males which had a Tₘₐₓ of 1.25 hours following administration. Plasma levels were relatively similar from 2.5 to 8 hours post-dose indicating that absorption was generally slow (no “dose-dumping”).

The systemic exposure (as measured by Cₘₐₓ and AUCₗₐₛₜ) of rats to bupivacaine increased with increasing dose over the dose range of 20 to 80 mg/kg, however, these increases were less than dose proportional. Overall, the Cₘₐₓ and AUCₗₐₛₜ values at the highest dose level (80 mg/kg) were approximately 57% and 25% lower, respectively, than values predicted from a linear relationship. Exposure was generally similar between males and females.

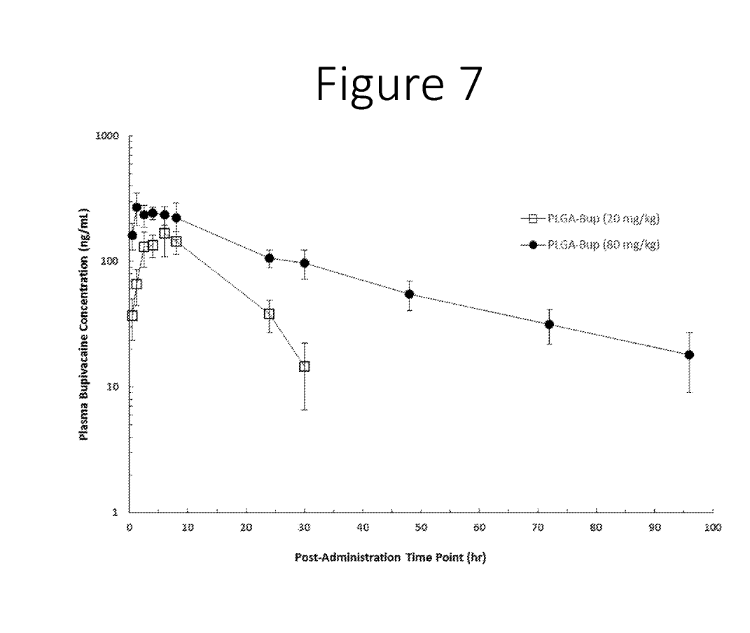

The terminal half-life could not be estimated adequately for all groups, but where estimated it appeared to be independent of sex and increased with increasing dose. The mean t_{1/2} increased approximately 4-fold with a 4-fold increase in dose; t_{1/2} was ˜6.6 and 27 hours for the 20 and 80 mg/kg dose levels (genders combined), respectively. Plasma bupivacaine concentration-time curves are shown in FIG. 7.

The rate and extent of systemic exposure of rats to bupivacaine appeared to be characterized by nonlinear (dose-dependent) kinetics following a single subcutaneous administration of over the dose range of 20 to 80 mg/kg. Increasing the dose of PLGA/bupivacaine particles above 20 mg/kg is likely to result in a lower systemic exposure than would be predicted from a linear relationship, which is consistent with dissolution-rate limited absorption of bupivacaine. As such, the Cₘₐₓ does not increase at an equivalent rate as AUC suggesting that PLGA/bupivacaine particles are able to provide a sustained exposure with a potentially improved tolerability profile. The toxicity of a single dose administered subcutaneously was also evaluated histologically in rats.

Sprague-Dawley rats (N=20/sex/group main study; N=3/sex/control and 6/sex/test article group for TK cohort) received a single subcutaneous administration of vehicle 1072-7, placebo particle 914-95-4, 20, or 80 mg/kg 1031-12 in a GLP study designed to evaluate the local toxicity and toxicokinetics (TK) of the test article. In the main study groups, 5/sex/group were euthanized on Days 7, 14, 30, and 60. Blood samples were collected from TK study groups at 0.5, 1.25, 2.5, 4, 6, 8, 24, 30, 48 and 72 hours following subcutaneous administration on Day 1.

For histological examination, 5 animals/sex/group were euthanized on Days 7, 14, 30, and 60. Toxicity was assessed by mortality, moribundity, clinical observations, body weights, and food consumption. At necropsy clinical pathology, organ weights, macroscopic observations, and microscopic pathology of the injection sites and draining inguinal lymph nodes were evaluated.

In the study, there were no test article related effects on mortality, moribundity, clinical observations (except at the injection site), body weights, and food consumption. Clinical observations included transient edema and raised areas localized to the site of injection. At necropsy, there were no test article related effects on clinical pathology, organ weights, and macroscopic observations.

Histopathological findings included reversible or reversing inflammatory reaction and healing response that were restricted to the subcutis. All injection site microscopic changes were localized to a small area within the superficial fibrous layer of the subcutis. Particle deposits were present at Days 7 and 14 in both PLGA/Bupivacaine particle groups as well as the PLGA placebo group. These particle deposits were more persistent at the 80 mg/kg dose level and the deposits were no longer observed at any of the injection sites on Days 30 or 60. Inflammatory response to the PLGA/Bupivacaine particles groups as well as the PLGA placebo group progressively decreased across Days 14, 30, and 60. This decrease coincided with the disappearance of the particles. The inflammatory response had completely resolved by Day 60 with resolution of the healing response (i.e. neovascularization, fibrosis, and fatty infiltration) almost complete. No change in draining inguinal lymph nodes were observed at Days 7, 14, 30, or 60.

### Example 9

Pharmacokinetic Evaluation of Bupivacaine Administered Subcutaneously in Yucatan Miniature Swine

A study was conducted to characterize the PK profile of bupivacaine in Yucatan miniature swine when administered subcutaneously as bupivacaine particles or PLGA/bupivacaine particles. Additionally, the PK profile was compared to the profiles of Exparel (bupivacaine liposome injectable suspension) (Pacira Pharmaceuticals, Inc., San Diego, Calif.) and bupivacaine hydrochloride solution (Marcaine, 0.75%).

Particles, 914-98-1, were manufactured in accordance with Example 1B with the following changes. The mold/film was passed through a laminator at 290° F. at 10 feet/minute. Particles were stored while in the mold for approximately 10 days prior to harvesting.

Particles, 914-98-2, were manufactured in accordance with Example 1A with the following changes. A 28 wt % solids homogeneous solution of 40 wt % PLGA (Evonik Industries, Resomer RG502H) and 60 wt % bupivacaine free base (Cayman Chemical Company) in acetone was prepared. The mold/film was passed through a laminator at 290° F. at 10 feet/minute. Particles were stored while in the mold for approximately 10 days prior to harvesting.

A vehicle, 1069-7, was also manufactured. First, a stock composition containing 0.82 wt % hyaluronic acid (1,000 kDa, Stanford, catalog HA-EP-1.8), 50 mM Tris (as Tris base and Tris HCl), 100 mM sodium chloride, 0.1 wt % polysorbate 80 was made. The stock composition was autoclaved. The viscosity of the stock composition was approximately 600-750 cps. Second, a diluting composition containing 50 mM Tris (as Tris base and Tris HCl), 100 mM sodium chloride, 0.1 wt % polysorbate 80 was made. The pH of the diluting composition was adjusted to approximately 8. The diluting composition was sterile filtered. The stock composition was diluted with the diluting composition by combining 85 wt % stock composition and 15 wt % diluting composition. The composition of the vehicle, 1069-7, was 0.7 wt % hyaluronic acid (1,000 kDa, Stanford, Catalog HA-EP-1.8), 50 mM Tris, 100 mM NaCl, 0.1 wt % polysorbate 80. The vehicle was sterilized by autoclave. The pH of the vehicle was approximately 8 and the viscosity was approximately 360 cps.

Prior to injection, vehicle was added to the particles to form a suspension for dosing. Vehicle was added to achieve the dosing concentrations in the table below. To form the suspension, vehicle was added to a vial containing the particles. The vial was vortexed for approximately 15 seconds. After vortexing, the vial was sonicated for approximately 15 seconds. The vial was vortexed/sonicated for 3-6 cycles until a uniform suspension was formed.

In the study, Yucatan miniature swine (N=3 males/group) received a single SC injection of Exparel (bupivacaine liposome injectable suspension), Marcaine, 914-98-2, or 914-98-1 as described in the table below.

| | Dose Level | Dose | Dose Volume |
|---|---|---|---|
| Test Article | (mg/kg) | (mg/mL) | (mL/kg) |
| Exparel | 4 | 13.3 | 0.3 |
| Marcaine | 2 | 5.0 | 0.4 |
| 914-98-2 | 2 | 6.7 | 0.3 |
| | 4 | 13.3 | 0.3 |
| | 6 | 20.0 | 0.3 |
| 914-98-1 | 2 | 6.7 | 0.3 |
| | 4 | 13.3 | 0.3 |
| | 6 | 20.0 | 0.3 |

Blood samples for PK analysis were collected 5 and 30 minutes and 1, 2, 4, 6, 8, 12, 24, 48, 72, 96, and 120 hours after dosing. Plasma concentrations of bupivacaine were determined using LC-MS/MS with a measurement range from 0.500-500 ng/mL and PK analysis was performed using WinNonlin.

Plasma bupivacaine exposure increased with increasing dose for both 914-98-2 and 914-98-1, with the increase for Cₘₐₓ and AUCₗₐₛₜ roughly dose proportional. A 1:2:3-fold increase in the 914-98-2 dose resulted in a 1:1.7:2.9-fold increase in Cₘₐₓ and a 1:1.6:3.0-fold increase in AUCₗₐₛₜ. Similarly, a 1:2:3-fold increase in the 914-98-1 dose was characterized by a 1:1.4:1.8-fold increase in bupivacaine Cₘₐₓ and a 1:1.8:3.0-fold increase in AUCₗₐₛₜ. The mean t₁₂ was dose independent ranging from 9.4 to 14.8 hours for both 914-98-2 and 914-98-1. Similarly, mean Tₘₐₓ was dose independent for 914-98-2 and 914-98-1. The mean Tₘₐₓ values for 914-98-1 ranged from 4.0 to 5.3 hours. The mean Tₘₐₓ demonstrated more variability for 914-98-2 ranging from 0.8 to 4.8 hours, but tended to be of shorter duration than 914-98-1. The variability noted for 914-98-2 at the 4 mg/kg dose was due a single animal exhibiting a Tₘₐₓ of 12 hours, which when eliminated, resulted in a mean Tₘₐₓ of 1.3 hours.

The kinetic profile for bupivacaine was generally similar, with the exception of a difference for Tₘₐₓ, for 914-98-2 and 914-98-1. The kinetic profile for 914-98-2 and 914-98-1 differed when compared to bupivacaine solution and liposomal bupivacaine.

At the 2 mg/kg dose, the bupivacaine solution exhibited a Tₘₐₓ that was 10-fold shorter and a Cₘₐₓ that was approximately 3-fold higher compared to 914-98-2. When compared to 914-98-1, bupivacaine solution exhibited a Tₘₐₓ that was approximately 66-fold shorter and a Cₘₐₓ that was approximately 2-fold higher.

At the 4 mg/kg dose, Exparel (bupivacaine liposome injectable suspension) exhibited a Tₘₐₓ that was comparable to the bupivacaine solution dosed at 2 mg/kg. Exparel (bupivacaine liposome injectable suspension) exhibited a Tₘₐₓ that was approximately 50- to 60-fold shorter than the Tₘₐₓ for 914-98-1 and 914-98-2. In addition, the mean t_{1/2} was approximately 2- to 2.5-fold longer.

At the 6 mg/kg dose, the Cₘₐₓ for 914-98-2 and 914-98-1 were approximately equal.

Data is summarized in the table below. Results are expressed as mean±standard deviation. N=3 for each group tested.

| Dose | | Cₘₐₓ | Tₘₐₓ | t_{1/2} | AUC₀₋₂₄ | AUCₗₐₛₜ |
|---|---|---|---|---|---|---|
| (mg/kg) | Treatment | (ng/mL) | (h) | (h) | (h*ng/mL) | (h*ng/mL) |
| 2 | 914-98-2 | 174 ± 48 | 0.8 ± 0.3 | 14.8 ± 5.9 | 2050 ± 196 | 2780 ± 262 |
| | 914-98-1 | 265 ± 75 | 5.3 ± 2.3 | 11.6 ± 5.2 | 2760 ± 1207 | 3530 ± 1697 |
| | Marcaine | 545 ± 232 | 0.08 ± 0 | 19.7 ± 2.9 | 1800 ± 664 | 3010 ± 991 |
| 4 | 914-98-2 | 297 ± 111 | 4.8 ± 6.3 | 10.7 ± 1.3 | 3070 ± 426 | 4400 ± 730 |
| | 914-98-1 | 381 ± 67 | 4.2 ± 3.8 | 9.4 ± 3.6 | 4600 ± 262 | 6250 ± 1148 |
| | Exparel | 146 ± 52 | 0.08 ± 0 | 24.4 ± 10.6 | 1920 ± 451 | 5050 ± 206 |
| 6 | 914-98-2 | 511 ± 198 | 1.5 ± 0.9 | 13.7 ± 2.8 | 6360 ± 2014 | 8320 ± 2178 |
| | 914-98-1 | 466 ± 179 | 4.0 ± 3.5 | 16.0 ± 3.8 | 6520 ± 2041 | 10700 ± 2220 |

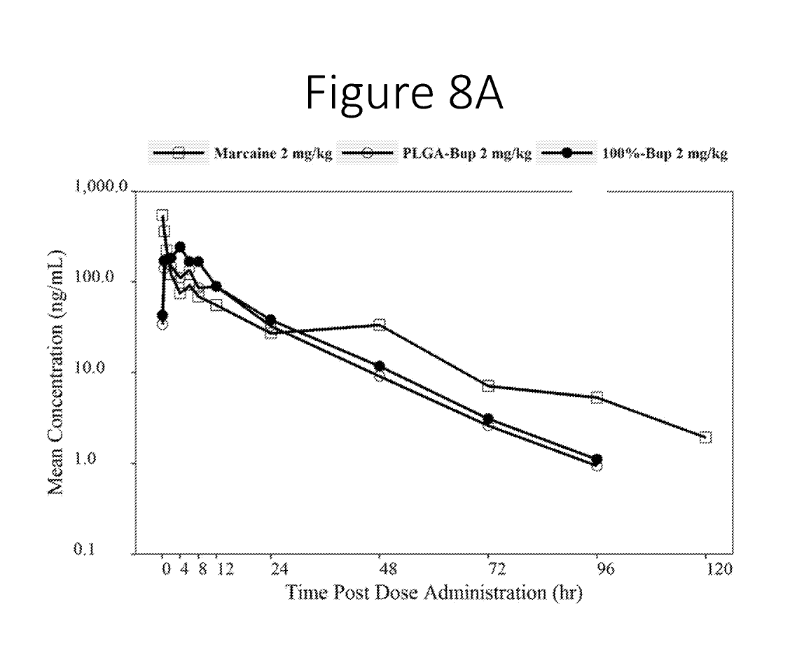

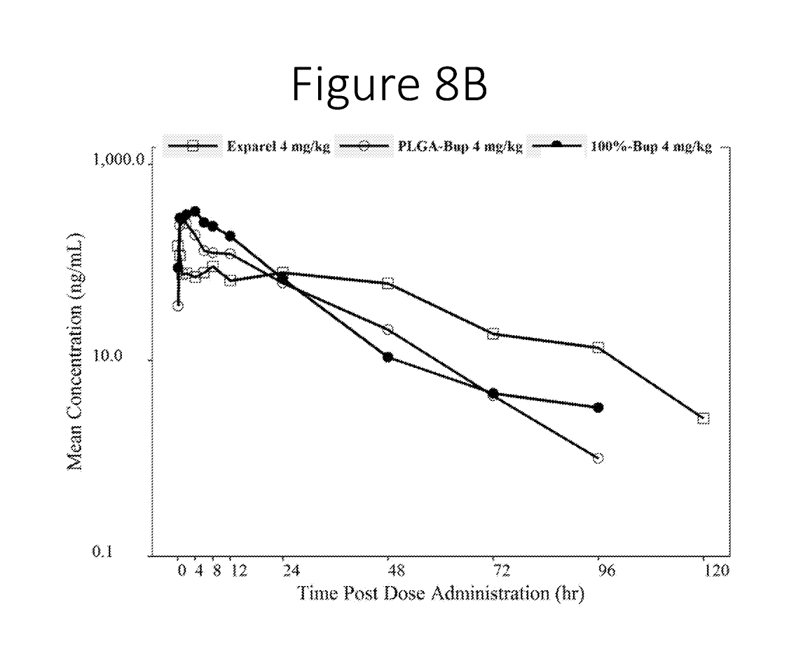

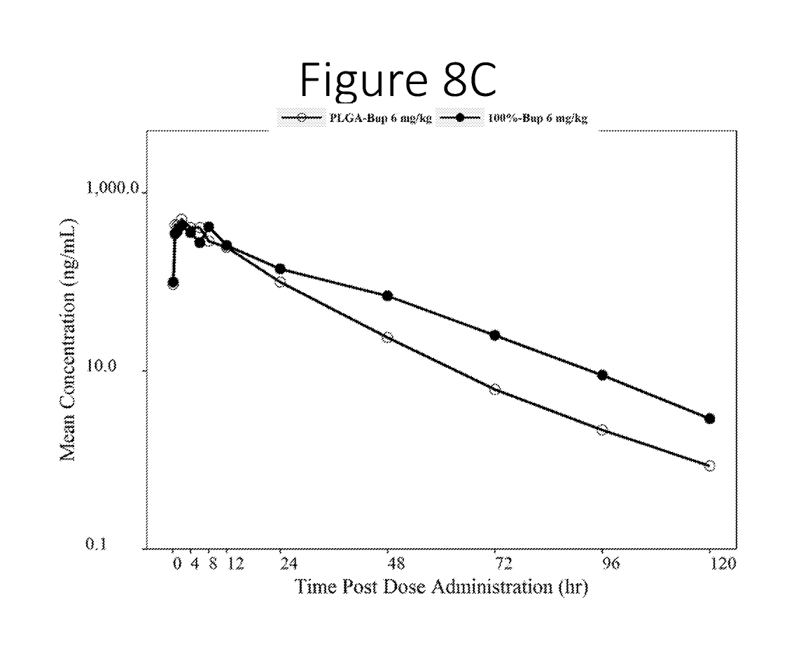

The plasma concentrations of bupivacaine for all test articles vs. time are illustrated in FIGS. 8A, 8B, and 8C. FIG. 8A depicts the mean bupivacaine plasma concentration (ng/mL) for the 2 mg/kg dose. FIG. 8B depicts the mean bupivacaine plasma concentration (ng/mL) for the 4 mg/kg dose. FIG. 8C depicts the mean bupivacaine plasma concentration (ng/mL) for the 6 mg/kg dose.

Thus the pharmacokinetics of the 914-98-2 and 914-98-1 formulations were comparable in the pig with the only difference being the time to peak effect. It is notable that 914-98-2 and 914-98-1 at 6 mg/kg provide peak concentrations below that of soluble bupivacaine hydrochloride.

### Example 10

Incisional Swine Example Using PLGA/Bupivacaine Particles

A study was conducted to determine the pharmacokinetic (PK) profile of bupivacaine when administered as PLGA/Bupivacaine particles suspended in vehicle or as Exparel (bupivacaine liposome injectable suspension) in Yucatan miniature swine.

PLGA/Bupivacaine particles, 1110-161, were manufactured in accordance with Example 1A with the following changes. A 28 wt % solids homogeneous solution of 40 wt % PLGA (Evonik Industries, Resomer RG502H) and 60 wt % bupivacaine free base (Cambrex) in acetone was prepared. The mold/film was passed through a laminator at 290° F. at 10 feet/minute. Particles were stored in the mold for approximately 7 days prior to harvesting.

A vehicle, 1072-22, was manufactured. First, a stock composition containing 1.0 wt % hyaluronic acid (1,000 kDa, Stanford, catalog HA-EP-1.8), 50 mM Tris (as Tris base and Tris HCl), 100 mM sodium chloride, 0.1 wt % polysorbate 80 was made. The stock composition was autoclaved. The viscosity of the stock composition was approximately 2000 to 3500 cps. Second, a diluting composition containing 50 mM Tris (as Tris base and Tris HCl), 100 mM sodium chloride, 0.1 wt % polysorbate 80 was made. The pH of the diluting composition was adjusted to approximately 8. The diluting composition was sterile filtered. The stock composition was diluted with the diluting composition. The vehicle had a final composition of 0.61 wt % hyaluronic acid (1,000 kDa, Stanford, catalog HA-EP-1.8), 50 mM Tris (as Tris base and Tris HCl), 100 mM sodium chloride, 0.1 wt % polysorbate 80. The pH was approximately 8.2. The viscosity was approximately 322 cps.

Prior to injection, the particles were suspended in the vehicle according to methods described herein. Vehicle was added to achieve the dosing concentrations shown in the table below. The table below details bupivacaine concentrations of the suspensions and the bupivacaine doses.

| | | | | Dose | Dose | Dose |
|---|---|---|---|---|---|---|
| | No. of | Dose | Test | Level | Concentration | Volume |
| Group | Animals | Route | Material | (mg/kg) | (mg/mL) | (mL/kg) |
| 1 | 3 M/3 F | SQ | 1110-161 | 6 | 15 | 0.4 |
| 2 | 3 M/3 F | | | 18 | 45 | 0.4 |
| 3 | 3 M/3 F | | | 36 | 90 | 0.4 |
| 4 | 3 M/3 F | | Exparel | 6 | 13.3 | 0.45 |

Following an acclimation period of at least 7 days, 24 Yucatan miniature swine (12 male and 12 female) were assigned to one of four treatment groups. On Study Day 1, prior to surgery, food-fasted animals received a single dose of Telazol and Xylazine (2.2 mg/kg IM) and ketoprofen (2.0 mg/kg IM). The animals were induced and maintained with direct administration of isoflurane (0.5-5% in 100% oxygen) via inhalation.

Once anesthetized, the left side of the neck and the left dorsal region of each animal was clipped free of hair using electric clippers and prepared for the incision using alternating chlorhexidine and alcohol wipes. The surgical area was then draped.

Each animal had a full-thickness incisional wound created along its dorsum, perpendicular to the midline. Following wound creation, a single dose of PLGA/Bupivacaine particles in vehicle or Exparel (bupivacaine liposome injectable suspension) was subcutaneously (SQ) injected through the wound tissue. Approximately half of the total volume was administered on each side of the incision equidistant from each edge of the wound. Prior to injection, the syringe plunger was drawn back to ensure the needle had not penetrated a blood vessel.

Parameters in the table below were monitored during the study.

| Parameters | Intervals |
|---|---|
| Mortality Observation | At least twice daily (AM & PM) |
| Physical Examination | During acclimation for assignment to study |
| Body Weights | Once during acclimation prior to dose |
| | administration |

Blood samples were collected pre-dose and at 5, 15, and 30 minutes; and 1, 2, 4, 8, 24, 48, 72, 96, 120, and 144 hours following dose administration. Pharmacokinetic (PK) modeling (Phoenix WinNonlin 6.4, Certara, Princeton, N.J.), was performed. The systemic exposure was evaluated from the plasma concentration—time curves based on the AUC, depending on dosage. The pharmacokinetic parameters of C_{MAX}, T_{MAX}, T_{LAST}, t_{1/2}, AUC_{LAST}, and AUC₀₋ₐ were estimated. C_{MAX}, T_{MAX}, and T_{LAST} were derived directly from the concentration—time results. In addition, dose linearity, clearance, and distribution were determined. The table below details the PK parameters.

| | | Cₘₐₓ | Tₘₐₓ* | Tₗₐₛₜ* | t_{1/2} | AUCₗₐₛₜ | AUC_{0-α} | Vz/F | Cl/F |
|---|---|---|---|---|---|---|---|---|---|
| Treatment | Sex | (ng/mL) | (hr) | (hr) | (hr) | (hr*ng/mL) | (hr*ng/mL) | (mL/kg) | (mL/hr/kg) |
| Exparel | M | 224 ± 115 | 5 nnin | 72 | 8.3 ± 2.3 | 6890 ± 1870 | 7890 ± 1260 | 9380 ± 4000 | 770 ± 123 |
| (6 mg/kg) | F | 443 ± 245 | 5 min | 72 | 19.8 ± 8.6 | 9380 ± 3360 | 9980 ± 3820 | 21600 ± 18100 | 673 ± 288 |
| LIQ865A | M | 359 ± 258 | 2.0 | 48 | 9.0 ± 3.5 | 5940 ± 2020 | 6020 ± 2030 | 15700 ± 12400 | 1100 ± 461 |
| (6 mg/kg) | F | 865 ± 345 | 2.0 | 72 | 8.3 ± 1.9 | 11000 ± 3010 | 9480 ± 1570 | 7790 ± 3000 | 642 ± 106 |
| LIQ865A | M | 604 ± 224 | 1.0 | 120 | 18.2 ± 7.1 | 15500 ± 3430 | 15700 ± 3430 | 31200 ± 14700 | 1190 ± 247 |
| (18 mg/kg) | F | 1450 ± 247 | 2.0 | 144 | 13.8 ± 3.7 | 33400 ± 6520 | 33500 ± 6480 | 11400 ± 5180 | 551 ± 107 |
| LIQ865A | M | 620 ± 110 | 2.0 | 144 | 20.0 ± 4.8 | 24200 ± 4070 | 24600 ± 3890 | 44200 ± 17400 | 1490 ± 240 |
| (36 mg/kg) | F | 1920 ± 326 | 1.0 | 144 | 32.9 ± 6.9 | 59300 ± 5790 | 61100 ± 5080 | 28400 ± 8370 | 592 ± 51.2 |

Mean ± SD;
*Median values.
M: male;
F: female.

Gender differences in bupivacaine systemic exposure were evident at all three dose levels of PLGA/Bupivacaine particles and Exparel (bupivacaine liposome injectable suspension). Female swine demonstrated a greater bupivacaine systemic exposure than the males. Within each gender, the SQ administration of Exparel (bupivacaine liposome injectable suspension) at 6 mg/kg resulted in the lowest bupivacaine mean Cₘₐₓ. At 6 mg/kg, Exparel (bupivacaine liposome injectable suspension)® and PLGA/Bupivacaine particles produced similar systemic bupivacaine AUCs with each possessing similar t_{1/2}s. Bupivacaine Tₘₐₓ was N5 minutes for Exparel (bupivacaine liposome injectable suspension) and ranged from 1 to 2 hours for the PLGA/Bupivacaine particle preparations. In male swine, there was a dose-related increase in bupivacaine mean AUCₗₐₛₜ and AUC_{0-α}, however the bupivacaine mean Cₘₐₓ was similar between the PLGA/Bupivacaine particle 18 and 36 mg/kg doses. In female swine, there was a dose proportional increase in systemic bupivacaine exposure as evidenced by mean AUCₗₐₛₜ and AUC_{0-α}. In both male and female swine, there was a dose associated increase in mean bupivacaine t_{1/2} with increasing PLGA/Bupivacaine particle dose.

Following a single subcutaneous administration of PLGA/Bupivacaine particles around a full-thickness incisional wound in Yucatan miniature swine, the slow controlled release of bupivacaine was demonstrated by the lack of an initial bupivacaine bolus release into systemic circulation, lack of an increasing Cₘₐₓ with a dose ≥18 mg/kg, a prolonged time to reach peak plasma levels and an increasing t_{1/2} with increasing dose.

A single, subcutaneous injection of Exparel (bupivacaine liposome injectable suspension) at 6 mg/kg around a full-thickness incision in Yucatan miniature swine resulted in a lower Cₘₐₓ but early Tₘₐₓ compared to the PLGA/Bupivacaine particle formulations.

### Example 11

Incisional Swine Example Using Bupivacaine Particles

A study was conducted to determine the pharmacokinetic (PK) profile of bupivacaine when administered as Bupivacaine particles suspended in vehicle or as Marcaine® in Yucatan miniature swine.

Bupivacaine particles, 1110-162, were manufactured in accordance with Example 1B with the following changes. The mold/film was passed through a laminator at 300° F. at 10 feet/minute. Particles were stored in the mold for approximately 7 days prior to harvesting.

Vehicle, 1072-22, as described above was also used in this study.

Prior to injection, the particles were suspended in the vehicle according to methods described herein. Vehicle was added to achieve the dosing concentrations shown in the table below. The table below details bupivacaine concentrations of the suspensions and the bupivacaine doses.

| | | | | Dose | Dose | Dose |
|---|---|---|---|---|---|---|
| | No. of | Dose | Test | Level | Concentration | Volume |
| Group | Animals | Route | Material | (mg/kg) | (mg/mL) | (mL/kg) |
| 1 | 3 M/3 F | SQ | 1110-162 | 6 | 15 | 0.4 |
| 2 | 3 M/3 F | | | 18 | 45 | 0.4 |
| 3 | 3 M/3 F | | | 36 | 90 | 0.4 |
| 4 | 3 M/3 F | | Marcaine ® | 2 | 5 | 0.4 |

Following an acclimation period of at least 7 days, 24 Yucatan miniature swine (12 male and 12 female) were assigned to one of four treatment groups. On Study Day 1, prior to surgery, food-fasted animals received a single dose of Telazol and Xylazine (2.2 mg/kg IM) and ketoprofen (2.0 mg/kg IM). The animals were induced and maintained with direct administration of isoflurane (0.5-5% in 100% oxygen) via inhalation.

Each animal had a temporary indwelling catheter placed into the jugular vein on the left side of the neck via cut-down method. The catheter was then exteriorized on the dorsal neck and sutured in place with appropriate sutures. Gauze was placed over the incision site and held in place with appropriate bandage material. The temporary catheter was used for blood collection procedures.

Each animal had a full-thickness incisional wound created along its dorsum near the midline. Following wound creation, a single dose of Bupivacaine particles in vehicle or Marcaine was subcutaneously (SQ) injected through the wound tissue. Parameters in the table below were monitored during the study.

| Parameters | Intervals |
|---|---|
| Mortality Observation | At least twice daily (AM & PM) |
| Physical Examination | During acclimation for assignment to study |
| Body Weights | Once during acclimation prior to dose |
| | administration |

Blood samples were collected pre-dose and at 5, 15, and 30 minutes; and 1, 2, 4, 8, 24, 48, 72, 96, 120, and 144 hours following dose administration. Pharmacokinetic (PK) modeling (Phoenix WinNonlin 6.4, Certara, Princeton, N.J.), was performed. The systemic exposure was evaluated from the plasma concentration—time curves based on the AUC, depending on dosage. The pharmacokinetic parameters of C_{MAX}, T_{MAX}, T_{LAST}, t_{1/2}, AUC_{LAST}, and AUC₀₋ₐ were estimated. C_{MAX}, T_{MAX}, and T_{LAST} were derived directly from the concentration—time results. In addition, dose linearity, clearance, and distribution were determined. The table below details the PK parameters.

| | | Cₘₐₓ | Tₘₐₓ* | Tₗₐₛₜ* | t_{1/2} | AUCₗₐₛₜ | AUC_{0-α} | Vz/F | Cl/F |
|---|---|---|---|---|---|---|---|---|---|
| Treatment | Sex | (ng/mL) | (hr) | (hr) | (hr) | (hr*ng/mL) | (hr*ng/mL) | (mL/kg) | (mL/hr/kg) |
| Marcaine ® | M | 984 ± 162 | 5 min | 48 | 9.5 ± 4.1 | 3670 ± 432 | 3780 ± 410 | 7310 ± 3130 | 533 ± 57.5 |
| (2 mg/kg) | F | 1080 ± 180 | 5 min | 48 | 8.0 ± 2.3 | 3830 ± 843 | 3930 ± 857 | 5990 ± 2140 | 524 ± 103 |
| LIQ865B | M | 575 ± 27.4 | 1.0 | 72 | 10.1 ± 3.5 | 6560 ± 54.7 | 6610 ± 35.1 | 13200 ± 4480 | 907 ± 4.81 |
| (6 mg/kg) | F | 573 ± 56.3 | 4.0 | 48 | 9.9 ± 1.9 | 9890 ± 676 | 10100 ± 762 | 8400 ± 1060 | 594 ± 46.6 |
| LIQ865B | M | 561 ± 21.1 | 2.0 | 96 | 9.1 ± 1.1 | 18200 ± 2390 | 18300 ± 2440 | 12900 ± 1310 | 996 ± 132 |
| (18 mg/kg) | F | 805 ± 203 | 1.0 | 96 | 9.3 ± 0.1 | 21400 ± 1810 | 20700 ± 1610 | 11700 ± 797 | 870 ± 67.5 |
| LIQ865B | M | 688 ± 13.7 | 2.0 | 120 | 13.5 ± 7.3 | 33200 ± 1070 | 33500 ± 1270 | 20800 ± 10500 | 1080 ± 41.2 |
| (36 mg/kg) | F | 1100 ± 87.4 | 1.0 | 144 | 11.0 ± 3.0 | 50000 ± 6380 | 50200 ± 6200 | 11600 ± 4120 | 724 ± 87.6 |

All animals were successfully administered the appropriate doses of their respective treatments, according to the study design and there were no observed adverse reactions during dosing. On Day 2, there were multiple instances of emesis and/or diarrhea noted in animals from the low dose Bupivacaine particle group, high dose Bupivacaine particle group, and the Marcaine group, primarily. However, there was no animal mortality observed during this study. All recorded animal body weights were considered to be within normal ranges for young laboratory swine. Bupivacaine systemic exposure and pharmacokinetics were determined, after a single SQ dose of Marcaine® dosed at 2 mg/kg or Bupivacaine particles dosed at 6, 18, or 36 mg/kg. Gender differences in bupivacaine systemic exposure were evident at all 3 dose levels of Bupivacaine particles. The plasma bupivacaine terminal half-life of ˜10 hours was similar across all formulations and doses.

Marcaine® was characterized by a rapid Tₘₐₓ, a greater Cₘₐₓ and lower AUCs than all Bupivacaine particle doses. There was little evidence of a Bupivacaine particle dose-related increase in bupivacaine mean Cₘₐₓ in male swine. However, there was a Bupivacaine particle dose related increase in systemic bupivacaine exposure, as measured by AUCₗₐₛₜ and AUC_{0-α}, in both male and female swine. A single SQ dose of Bupivacaine particles at all dose levels resulted in greater systemic bupivacaine exposure than Marcaine®.

In conclusion, a single, subcutaneous (SQ) injection of Bupivacaine particles dosed at 6, 18, and 36 mg/kg around a full-thickness incision in Yucatan miniature swine resulted in dose proportional increases in exposure with females having a higher systemic exposure than males. A single, subcutaneous (SQ) injection of Marcaine® at 2 mg/kg around a full-thickness incision in Yucatan miniature swine resulted in rapid absorption with low systemic exposure. Though plasma concentrations of Marcaine® was greater than and/or equal to that produced by Bupivacaine particle formulations, the systemic exposure of bupivacaine delivered as Marcaine was not to the extent of the Bupivacaine particle formulations.

### Example 12

A Maximum Tolerated Dose (MTD) Study in Rats

Studies were conducted to determine the maximum tolerated dose (MTD) for PLGA/bupivacaine particles and bupivacaine particles in male and female Sprague-Dawley rats following a single SC injection.

12.1. PLGA/Bupivacaine Particles

Particles, 976-27-1, were manufactured in accordance with Example 1A with the following changes. A 28 wt % solids homogeneous solution of 40 wt % PLGA (Evonik Industries, Resomer RG502H) and 60 wt % bupivacaine free base (Cayman Chemical Company) in acetone was prepared. Particles were stored while in the mold at approximately 40° C./25% relative humidity for approximately 12 days prior to harvesting.

A vehicle, 1019-22-1, was manufactured for use in the study. First, a stock composition containing 1.0 wt % hyaluronic acid (1,000 kDa, Stanford, catalog HA-EP-1.8), 50 mM Tris (as Tris base and Tris HCl), 100 mM sodium chloride, 0.1 wt % polysorbate 80 was made. The stock composition was autoclaved. The viscosity of the stock composition was approximately 2000 to 3500 cps. Second, a diluting composition containing 50 mM Tris (as Tris base and Tris HCl), 100 mM sodium chloride, 0.1 wt % polysorbate 80 was made. The pH of the diluting composition was adjusted to approximately 8. The stock composition was diluted with the diluting composition by combining 75 wt % stock composition and 25 wt % diluting composition. The final composition of the vehicle was 0.75 wt % hyaluronic acid (1,000 kDa, Stanford, catalog HP-EP-1.8), 50 mM Tris (as Tris base and Tris HCl), 100 mM sodium chloride, 0.1 wt % polysorbate 80. The pH of the vehicle was approximately 8. The viscosity of the vehicle was approximately 814 cps.

Prior to injection, the particles were suspended in vehicle. Vehicle was added to achieve the dosing concentrations in the table below. To form the suspension, vehicle was added to a vial containing the particles. The vial was vortexed for approximately 30 seconds. After vortexing, the vial was sonicated for approximately 30 seconds. The vial was vortexed/sonicated for 3 cycles until a uniform suspension was formed. After aspirating the desired volume into a syringe, the suspension was further mixed by syringe to syringe mixing through a female-female luer lock connector.

| | | | |
|---|---|---|---|
| | Bupivacaine | Volume | Number of |
| Dose | Concentration | per dose | animals |

| | | | | |
|---|---|---|---|---|
| (mg/kg) | (mg/mL) | (mL/kg) | Male | Female |

Phase 1

| | | | | |
|---|---|---|---|---|
| 400 | 100 | 4 | 3 | 0 |
| 540 | 135 | 4 | 3 | 0 |
| 200 | 50 | 4 | 3 | 0 |
| 300 | 75 | 4 | 3 | 0 |

Phase 2

| | | | | |
|---|---|---|---|---|
| 40 | 26.8 | 1.5 | 3 | 3 |
| 80 | 53.5 | 1.5 | 3 | 3 |

Premature death/euthanasia was observed in male rats at doses of ≥200 mg/kg. There were no gross observations, apart from findings at the injection site, in the premature decedents. There were no apparent test article-related findings on body weight. Clinical signs, consistent with bupivacaine-induced seizure activity reported in the literature, included convulsions, head-bobbing, teeth-chattering and/or muscle spasms in males at a dose of ≥200 mg/kg. A single male at 40 mg/kg exhibited transient head bobbing and teeth chattering for 3 hours beginning 35 minutes after administration. The MTD was considered to be 80 mg/kg in males and at least 80 mg/kg in females.

12.2. Bupivacaine Particles

Particles, 914-95-1, were manufactured in accordance with Example 1B with the following changes. Particles were stored while in the mold at ambient conditions for approximately 11 days prior to harvesting.

Prior to injection, vehicle was added to the particles to form a suspension for dosing. Vehicle was added to achieve the dosing concentrations in the table below. To form the suspension, vehicle was added to a vial containing the particles. The vial was vortexed for approximately 30 seconds. After vortexing, the vial was sonicated for approximately 30 seconds. The vial was vortexed/sonicated for 3 cycles until a uniform suspension was formed. After aspirating the desired volume into a syringe, the suspension was further mixed by syringe to syringe mixing through a female-female luer lock connector.

| | | | |
|---|---|---|---|
| | Bupivacaine | Volume | Number of |
| Dose | Concentration | per dose | animals |

| (mg/kg) | (mg/mL) | (mL/kg) | Male | Female |
|---|---|---|---|---|
| 500 | 100 | 5 | 3 | 0 |
| 750 | 150 | 5 | 3 | 0 |
| 500 | 100 | 5 | 0 | 3 |
| 300 | 60 | 5 | 0 | 3 |
| 40 | 26.67 | 1.5 | 3 | 3 |
| 80 | 53.33 | 1.5 | 3 | 3 |
| 160 | 106.7 | 1.5 | 3 | 3 |

Animals were euthanized 24 to 72 hours following dose administration.

All males survived to scheduled euthanasia, whereas females were euthanized for humane reasons (i.e., due to prolonged seizure activity) for doses ≥300 mg/kg. Clinical signs, consistent with bupivacaine-induced seizure activity reported in the literature, included convulsions, head-bobbing, teeth-chattering and muscle spasms and were seen in males at a dose of 750 mg/kg. One of 3 females dosed at 40 mg/kg had a transient, mild convulsion lasting 30 seconds. The female at 40 mg/kg also exhibited a short whole body muscle spasm at 256 minutes following test article administration. All other females dosed at 40, 80, or 160 mg/kg were within normal limits throughout the observation period. Injection site observations were reported for females that underwent gross necropsy (300 and 500 mg/kg dose groups) and included a cyst-like structure (containing white fluid consistent with test article) and associated edema. The MTD in females was 160 mg/kg and in males was 500 mg/kg.

### Example 13

A Single-Dose Pilot Local Tolerance and Biocompatibility Study in Rats

Local tolerance was assessed in Sprague-Dawley rats for both PLGA/bupivacaine particles and bupivacaine particles.

The same materials utilized in previous studies were also used in the local tolerance and biocompatibility study: 976-27-1, 914-95-1, 914-95-4, and 1019-22-1.

13.1. PLGA/Bupivacaine Particles

976-27-1 and 914-95-4 were both suspended using vehicle 1019-22-1 following the procedure detailed in the MTD study.

Local tolerance was assessed in Sprague-Dawley rats (N=9 males, 3/time point) administered placebo particles, 914-95-1, on the left side of the animal and 976-27-1 at 80 mg/kg on the right side. Animals were euthanized following a 15, 30, or 60 day observation period. Injection sites underwent microscopic assessment.

A bioreactivity rating was calculated by determining a score for each dose site based on the inflammatory reaction and healing response. For the inflammatory reaction, the score included cell type and severity (i.e., neutrophils, lymphocytes, plasma cells, macrophages, and giant cells), the severity of necrosis, and other relevant histopathological findings. The inflammatory response was weighted by a factor of 2. For the healing response, the degree of neovascularization, fibrosis, fatty infiltration, and other relevant microscopic changes were scored. The inflammatory and healing scores were added together to derive a total score for each animal. The bioreactivity rating was the difference in average scores between the 976-27-1 and placebo scores. A score ≤2.9 was considered no reaction, 3.0 to 8.9 a slight reaction, 9.0 to 15.0 a moderate reaction, and >15.0 a severe reaction.

On Day 15, the tissue response to 976-27-1 and placebo 914-95-4 was similar and included macrophages and giant cells with peripheral accumulations of lymphocytes. Giant cells and areas of necrosis were more common and/or more severe in the 976-27-1 sites compared to the placebo particle sites. All changes were limited to the subcutis. A bioreactivity score of 6 indicated that 976-27-1 was a slight irritant, based on the Day 15 response.

By Day 30, 976-27-1 injections sites had greater numbers of macrophages than the placebo sites, with a few of the macrophages containing non-staining intracytoplasmic vacuoles ranging in size from 10 to 40 μm. Particles consistent with the PLGA particles were not observed at the injection sites. Fibrosis, neovascularization and fatty infiltrates were more common and greater in severity in the placebo control than the 976-27-1 injection sites. Both control and test sites had minimal to moderate lymphocytes, rare plasma cells, and rare giant cells. All changes occurred within the subcutis. Overall scores for 976-27-1 and placebo 914-95-4 were comparable, resulting in a bioreactivity rating of 0 for 976-27-1 by Day 30.

Day 60 microscopic examination showed that the inflammation had resolved for both the placebo and 976-27-1 and that healing was almost complete for placebo and test article. There was no indication of PLGA presence at 60 days.

13.2. Bupivacaine Particles

914-95-1 was suspended using vehicle 1019-22-1 following the procedure detailed in the MTD study.

Local tolerance was assessed in Sprague-Dawley rats (N=9 males, 3/time point) administered vehicle control on the left side of the animal and 914-95-1 test article at 80 mg/kg on the right side. Animals were euthanized on Study Days 3, 7 or 14.

All animals survived to scheduled euthanasia. There were no test article-related effects on daily observations, clinical observations (excluding evaluation of the dose sites, body weights, and food consumption.

Test article-related effects were noted on daily observations and macroscopic and microscopic assessment, were localized, exhibited signs of recovery, and were considered non-adverse. The clinical observations included moderate edema and raised areas following administration of vehicle or 914-95-1 on Day 1 (the day of dose administration). Edema and raised areas resolved by Day 5 for the vehicle and by Day 7 (edema) or Day 13 (raised areas) for 914-95-1.

The microscopic changes on Days 3 and 7 following injection were associated with changes in the subcutis, including the presence of the test article, neutrophilic infiltrates, dilated congested blood vessels, increased edema, necrosis, inflammatory cell infiltrates and neovascularization/fibrosis. The incidence and/or severity decreased Day 7 compared to Day 3. Inflammatory cell infiltrates and edema were noted in the control dosed animals as well, although the incidence and severity were greater for the test article. Most changes had completely reversed by Day 14 following injection. All changes were localized to a small area within the superficial fibrous layer of the subcutis. A focus of slight neovascularization and fibrosis and slightly increased inflammatory cell infiltrates are likely to exhibit full reversal at a future date.

### Example 14

Maximum Tolerated Dose and Histopathological Evaluation in Yucatan Miniature Swine

A study was conducted to determine the maximum systemically tolerated dose and local tolerability of bupivacaine and PLGA/bupivacaine particles suspended in vehicle in Yucatan miniature swine.

PLGA/Bupivacaine particles, 914-122, were manufactured in accordance with methods disclosed herein for PLGA/bupivacaine drug particles. Bupivacaine particles, 914-123-1, were manufactured in accordance with methods disclosed herein for bupivacaine drug particles.

Prior to injection, each of the PLGA/bupivacaine drug particles and the bupivacaine drug particles were suspended in a vehicle having a viscosity greater than 50 cps as disclosed herein. Vehicle was added to achieve the dosing concentrations shown in the table below. The table below details bupivacaine concentrations of the suspensions and the bupivacaine doses.

| | | | | | Dose | Dose | Dose |
|---|---|---|---|---|---|---|---|
| | Dose | No. of | Dose | Test | Level | Conc. | Vol. |
| Cohort | Group | Animals | Route | Material | mg/kg | mg/mL | mL/kg |
| A | 1 | 1 M/1 F | SQ | 914-122 | 12 | 30 | 0.4 |
| | 2 | 1 M/1 F | | 914-122 | 24 | 60 | 0.4 |
| | 3 | 1 M/1 F | | 914-122 | 36 | 90 | 0.4 |
| B | 1 | 1 M/1 F | SQ | 914-123-1 | 12 | 30 | 0.4 |
| | 2 | 1 M/1 F | | 914-123-1 | 24 | 60 | 0.4 |
| | 3 | 1 M/1 F | | 914-123-1 | 36 | 90 | 0.4 |

Following an acclimation period of at least 7 days, 12 Yucatan miniature swine (6 male and 6 female) were assigned to one of two cohorts (A or B) with 3 groups (1, 2, 3) per cohort (1/gender/group). On Study Day 1, prior to surgery, food-fasted animals received a single dose of Telazol and Xylazine (2.2 mg/kg IM) and ketoprofen (2.0 mg/kg IM). The animals were induced and maintained with direct administration of isoflurane (0.5-5% in 100% oxygen) via inhalation. Once anesthetized, the dorsal region of each animal was clipped free of hair using electric clippers and prepared for the incision using alternating chlorhexidine and alcohol wipes followed by a final iodine spray. A single vertical (dorsal to ventral) incisional full thickness wound (˜8-10 cm) was created using a sterile scalpel blade. The drug particles suspended in vehicle were administered directly into the subcutaneous layer, then the incision was sutured closed. Each animal had a single incisional wound created and a single subcutaneous (SQ) dose administration. Dosing was escalated sequentially from Group 1 to Group 2 and from Group 2 to Group 3 based on the clinical observations of the earlier cohort. On Study Day 3, the incision site from 1 animal per group per cohort was collected for histopathological evaluation. On Study Day 14, the remaining animal, 1 animal/group/cohort, had the incision site collected for histopathological evaluation.

Parameters in the table below were monitored during the study.

| Parameters | Approximate Intervals |
|---|---|
| Mortality Observation | Twice daily |
| Physical Examination | During acclimation for assignment to study |
| Body Weights | During acclimation and prior to dose |
| | administration |
| Clinical Observations | Prior to dose administration and daily thereafter |
| Food Consumption | Once daily starting in acclimation, Quantitative |
| Histopathology (Incision | Day 3 (1 animal/cohort) and Day 14 (1 animal/ |
| site) | cohort) |

One animal from each dose group was euthanized and had the incision site (en bloc) collected on Day 3. The remaining animal was euthanized on Day 14 and had the incision site collected.

The incision sites were preserved in 10% neutral-buffered formal (NBF). Each incision site was sutured on the outer edges to a plastic card to ensure even fixation of the tissue.

Preserved incision sites were submitted to for histopathological evaluation. Preserved tissues were embedded in paraffin, sectioned, and stained with hematoxylin and eosin, and examined microscopically by a board certified pathologist.

On Day 3 post-surgery, microscopic examination showed morphological findings consistent with the procedure: acute inflammation, hemorrhage, and myofiber degeneration. In addition, microparticles associated with minimal to mild cellular infiltrates at ≥12 mg/kg for PLGA/Bupivacaine particles. By Day 14, for the specimens examined, all skin incisions had healed as determined by the presence of re-epithelialization over the incision line and fibrosis bridging the edges of the incision line. Additionally, granulomatous inflammation was observed with PLGA/Bupivacaine particles in the subcutis. Under the conditions of the study, both test samples were well tolerated at the application site at dose levels up to 36 mg/kg in this incisional wound healing skin model in Yucatan miniature swine.

In conclusion, both particle compositions suspended in vehicle proved to be well tolerated when administered subcutaneously around a full-thickness incision in Yucatan miniature swine at all dose levels tested. There were no clinical signs of illness or adverse reaction to the treatment noted during the study. Macroscopically and microscopically, the incision sites were healing at Day 3 or were healed by Day 14 demonstrating that both particle types were well tolerated locally. The MTD was determined to be >36 mg/kg. For 914-122 particles, 36 mg/kg was the maximum feasible dose (MFD) due to concentration and volume limitations for the dosing formulation.

### Example 15

Description of the Investigational Medicinal Products

This section describes two investigational medicinal products (IMPs) for use in clinical trials. Both IMPs are sterile dry powders that are reconstituted with a vehicle to produce an injectable suspension. One IMP is a powder derived from bupivacaine drug substance and the other is a powder derived from a bupivacaine drug substance/excipient mixture.

15.1 Bupivacaine Drug Particles:

Bupivacaine particles (214 mg) for injectable suspension, is hereinafter referred to as Bupivacaine particles and/or Bupivacaine particles drug product. These microparticles are composed of bupivacaine without additional excipients.

Bupivacaine particles are supplied as a sterile dry powder for single use which is diluted with a sterile vehicle and then mixed extemporaneously to give a homogeneous suspension prior to use. The drug product vial(s) and vehicle vial(s) are supplied separately. Two sterile plastic syringes and one sterile plastic connector, which are required to mix the suspension, will be provided by the Phase 1 unit. The final concentration of drug substance, bupivacaine, is dependent on the intended dose and can be formulated up to a maximum of 60 mg/mL with the vials provided. 10 mL will be administered by subcutaneous injection giving a maximum total bupivacaine dose of 600 mg. The clinical presentation, including allowances for overages, is described below.

Composition of Bupivacaine Particles Drug Product

| | Unit Quantity | |
|---|---|---|
| | and/or | |
| | percentage | |
| Ingredients/components | formula | Function/Role |
| Drug product | | |
| Bupivacaine | 214 mg* | Active ingredient |
| Other Components | | |
| Nitrogen | QS | Vial headspace |
| | | gas |
| Container closure system | | |
| 30 mL Clear Type 1 glass vial** | 1 | Container closure |
| 20 mm Igloo bromobutyl rubber | 1 | Container closure |
| stopper | | |
| Red Flip-off Aluminium cap | 1 | Container closure |

*223 mg of powder was filled into each vial. This quantity of powder includes adequate overage to allow for dosing at strengths ranging from 150 mg to 600 mg bupivacaine and administration of 10 mL of these strengths.
**After gamma irradiation, as expected, the vial becomes discolored.

Bupivacaine Particles: Studies using Bupivacaine particles demonstrated that particles of consistent size and shape are produced with D₅₀ values in the range of 25 μm. Furthermore, these particles, in a nitrogen environment, withstand gamma irradiation at 25 kGy and 45 kGy without impacting particle shape or size distribution, drug levels, or significantly increasing the levels of drug impurities. Thus, particles have a means of terminal sterilization. The stability characteristics of gamma irradiated Bupivacaine particles have been evaluated at −20° C. on prototype/development batches.

Furthermore, Bupivacaine particles administered in non-clinical studies subcutaneously to rats shows a pharmacokinetic profile which compares favourably to Exparel (bupivacaine liposome injectable suspension) (Pacira Pharmaceuticals, Inc., San Diego, Calif.). The results indicated a lower Cₘₐₓ and later Tₘₐₓ for the Bupivacaine particles and a higher area under the curve (AUC) over 72 hours compared to the Exparel (bupivacaine liposome injectable suspension) profile. In the figure below, Exparel (bupivacaine liposome injectable suspension) is represented by the square, and Bupivacaine particles are represented by the diamond.

15.2 PLGA/Bupivacaine Drug Particles:

PLGA/bupivacaine drug particles (244 mg), is hereinafter referred to as PLGA/bupivacaine particles and/or PLGA/bupivacaine drug product. These microparticles are composed about 55%-60% bupivacaine and 40%-45% poly(lactic-co-glycolic) acid (PLGA). The PLGA contains a 50/50 ratio of lactic to glycolic acid.

PLGA/bupivacaine particles are supplied as a sterile dry powder for single use which is diluted with a sterile vehicle and then mixed extemporaneously to give a homogeneous suspension prior to use. The drug product vial(s) and vehicle vial(s) are supplied separately. Two sterile plastic syringes and one sterile plastic connector, which are required to mix the suspension, will be provided by the Phase 1 unit. The final concentration of drug substance, bupivacaine, is dependent on the intended dose and can be formulated up to a maximum of 60 mg/mL with the vials provided. 10 mL will be administered by subcutaneous injection giving a maximum total bupivacaine dose of 600 mg. The clinical presentation, including allowances for overages, is described below.

Composition of PLGA/Bupivacaine Particles Drug Product

| | Unit Quantity | |
|---|---|---|
| | and/or | |
| | percentage | |
| Ingredients/components | formula | Function/Role |
| Drug product | | |
| Bupivacaine/PLGA | 244 mg* | Active |
| (55%/45% w/w) | bupivacaine | ingredient |
| | | combined with |
| | | PLGA which |
| | | improves |
| | | dispersion |
| | | during |
| | | suspension |
| Other Components | | |
| Nitrogen | QS | Vial headspace |
| | | gas |
| Container closure | | |
| 30 mL Clear Type 1 glass | 1 | Container |
| vial** | | closure |
| 20 mm Igloo bromobutyl | 1 | Container |
| rubber stopper | | closure |
| Blue Flip-off Aluminium cap | 1 | Container |
| | | closure |

*A powder consisting of bupivacaine and PLGA is produced from the PRINT process. 430 mg of this powder was filled into each vial. This quantity of powder includes adequate overage to allow for dosing at strengths ranging from 150 mg to 600 mg bupivacaine and administration of 10 mL of these strengths.
**After gamma irradiation, as expected, the vial becomes discolored.

PLGA/Bupivacaine Particles: Experiments on prototype particles demonstrated that the presence of PLGA had no appreciable effect on the pharmacokinetic profile for bupivacaine. In this instance for this configuration under these conditions, PLGA plays no significant role on the release rate of bupivacaine from the particles. However, resuspension experiments indicate that PLGA serves a role to facilitate dispersion of the particles in suspension.

A comparison of the pharmacokinetic profile of microparticles containing 55% Bupivacaine and 45% PLGA to Exparel (bupivacaine liposome injectable suspension) (Pacira Pharmaceuticals, Inc., San Diego, Calif.), following subcutaneous administration demonstrated a reduced Cₘₐₓ and a longer time to Tₘₐₓ with an equivalent AUC.

Investigational Medicinal Product Vehicle

The composition of the Vehicle is shown in the table below. The vehicle used for the suspension and injection of Bupivacaine particles and PLGA/bupivacaine particles is a sterile, clear, aqueous, isotonic, pH 8 solution which contains a viscosity modifier (sodium hyaluronate). Full details of the vehicle development, manufacture, control and stability are described below.

Composition of Vehicle

| | Unit Quantity | | |
|---|---|---|---|
| | and/or | | |
| | percentage | | |
| Ingredients/components | formula | | Function/Role |
| Sodium hyaluronate | 7.0-12.5 | mg/g | Viscosity modifier |
| Sodium chloride | 5.8 | mg/g | Tonicity modifier |
| Polysorbate 80 | 1.0 | mg/g | Surfactant |
| Tromomethamine (Tris | 6.1 | mg/g | Buffer |
| base) | | | |
| Hydrochloric acid | QS to pH 8 | | pH adjustment |
| Sterile Water for Injection | QS | | Solvent |
| Container closure | | | |
| 10 mL Clear Type 1 glass | 1 | | Container closure |
| vial | | | |
| Flurotec ® coated | 1 | | Container closure |
| bromobutyl rubber Serum | | | |
| Stopper | | | |
| Blue Flip-off Aluminium cap | 1 | | Container closure |
| Nominal volume | 8 | mL | n/a |

Dose Uniformity and Compatibility

The compatibility of the drug product with the Vehicle and the compatibility of this suspension with the mixing procedure were evaluated.

Studies demonstrated the target doses are achieved and that dose uniformity in the syringe is maintained when suspended following the clinical mixing protocol. Data from five dose preparations of both the 150 and 600 mg target bupivacaine doses demonstrated that target doses are achieved as shown in the following table.

| | Target Dose | Actual dose | |
|---|---|---|---|
| | (mg in | average (n = 5) mg | |
| Drug Product | 10 mL) | in 10 mL | % of target |
| Bupivacaine | 150 | 149 | 99.1 |
| particles | 600 | 567 | 94.5 |
| PLGA/bupivacaine | 150 | 166 | 111 |
| particles | 600 | 574 | 95.6 |

The following table demonstrates that the drug product suspension is homogeneous within the syringe used for administration.

| | | | 600 mg/10 mL |
|---|---|---|---|
| | | 150 mg/10 mL target | target dose |
| | | dose | % of target |
| | Location | % of target | concentration |
| Drug Product | in syringe | concentration (n = 3) | (n = 3) |
| Bupivacaine | beginning | 96.0 | 94.3 |
| particles | middle | 95.6 | 94.9 |
| | end | 95.7 | 95.1 |
| PLGA/bupivacaine | beginning | 94.0 | 93.2 |
| particles | middle | 94.3 | 94.3 |
| | end | 95.1 | 94.6 |

Studies showed compatibility between the particles and the vehicle when combined by following the clinical mixing protocol. In addition, no additional related substances were observed after resuspension or when the suspension is held in the syringe for a period of one hour at ambient room temperature.

### Example 16

Clinical Example

Randomized, controlled, Double-Blind, Single Ascending Dose Safety and Pharmacokinetic/Pharmacodynamic Study in Healthy Adult Males A clinical study was conducted to assess and characterized the safety and tolerability of particles of the invention compared to vehicle when infiltrated into a defined area of the medial calf of healthy adult Caucasian male subjects.

Hypo- and hyper-responders to thermal sensitivity testing were excluded from the study. A suprathreshold, short tonic, heat stimulus (STHS) consisting of 47° C. for 5 s duration using a contact thermode (50×25 mm²) applied at the non-dominant thigh (long axis in the midline, distal border of the thermode 15 cm above the superior margin of the knee cap, i.e. measured with the knee flexed 900. Subjects rated the perceived pain intensity using a numerical pain scale of 0 to 10 (numerical rating score (NRS)). Hypo-responders were defined as subjects who reported a NRS pain score of </=2/10 during STHS stimulation. Hyper-responders were defined as subjects who reported a NRS pain score of >/=8/10 during STHS stimulation.

Both pharmacokinetics (PK) and pharmacodynamic (PD) responses were evaluated in the study.

For pharmacokinetics, bupivacaine plasma PK after a single dose of particles of the invention was characterized. The individual plasma concentration/time curves and cohort mean PK parameters for each dosing cohort were determined. Blood samples were obtained at T=0, 0.5, 1, 1.5, 2, 2.5, 3, 3.5, 4, 5, 6, 8, 12, and 24 hours after administration. For 0.5, 1, and 1.5 hour PK assessments, the time window was +/−5 minutes. For the 2 through 6 hour, inclusive, PK assessments, the time window was +/−10 minutes. For the 8 through 24 hour, inclusive, PK assessments, the time window was +/−15 minutes. Plasma was analyzed for bupivacaine content. In addition, local and systemic safety assessments were conducted at the same time intervals.

Pharmacodynamic responses were evaluated using both thermal and mechanical stimulation tests performed within the defined area of the medial calf. Changes in sensory detection thresholds and pain thresholds from baseline for particles of the invention and the active comparator were assessed.

Thermal thresholds were assessed: warmth detection threshold (WDT), cold detection threshold (CDT), and heat pain threshold (HPT). The thermal thresholds were measured using a computerized thermode (active surface: 2.5×5.0 cm²; MSA, Somedic AB, Sweden) from a baseline temperature of 32° C. with a ramp rate of +/−1.0° C./s, and 50.0° C. and 5.0° C. as cut-off temperatures. Subjects pushed a button immediately when they experienced a change in temperature sensation (WDT and CDT) or when the heat stimulus was perceived as painful (HPT). After activation of the button, the thermode temperature returned to the baseline temperature. Assessments were made three times and the mean value was recorded for WDT and CDT. The median value was recorded for HPT.

Mechanical detection thresholds (MDT) were assessed using calibrated polyamide monofilaments (Stoelting-Europe, Dublin, Ireland; designated values 2.36 to 6.65 [bending force 0.2 to 447 mN]). MDTs provided information about the extent of mechanical tactile hypoesthesia. Subjects indicated when the smallest monofilament applied perpendicularly to the skin in the test area was perceived as a tactile sensation. The MDT was determined five time using monofilaments of ascending or descending order and using a modified Dixon algorithm. The median of the assessment was used for analysis.

Mechanical pain thresholds (MPT) were assessed using “weighted-pin” stimulators (PinPrick, MRC Systems, Heidelberg, Germany; 8-512 mN; tip-area 0.049 mm²). Subjects indicated when at least three of five perpendicularly applied pin-prick stimuli were perceived as painful. The pin-prick stimuli were applied evenly and in a random fashion. The MPT was determined five time using pin-prick stimulators of ascending or descending order and using a modified Dixon algorithm. The median of the assessment was used for analysis.

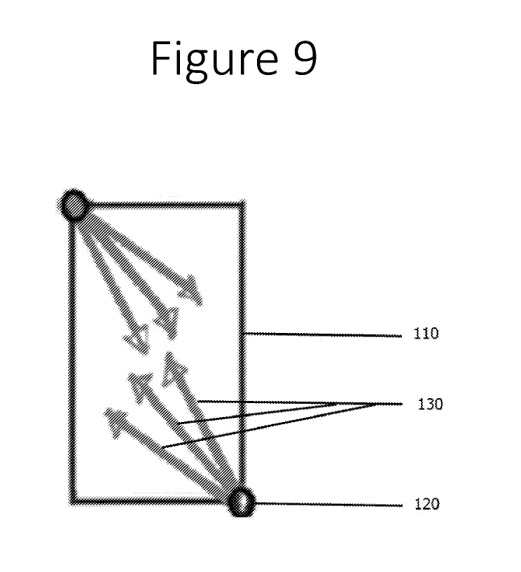

As shown in FIG. 9, a 60×35 mm rectangular area (long axis oriented vertically), 110, was delineated on each subject using a semi-permanent skin marker. The upper anterior corner of the rectangle was approximately 11 cm below the medial meniscus margin of the knee and approximately 6 cm from the anterior margin of the tibia. Opposite diagonal corners (upper lateral and lower medial), 120, were marked using a semi-permanent marker with open circles for administration of particles of the invention. Sensory testing was centered inside a 50×25 mm rectangle centered within the larger 60×35 mm rectangle.

Prior to injection, the marked rectangular area on the medical calf was inspected for evidence of infection or other abnormalities that may interfere with PD testing assessments. For the injection, the marked opposite diagonal corners were injected using a 2-inch, 21 gauge needle. 5 mL was delivered using a fanning technique as shown in FIG. 9. From each marked opposite diagonal corner, 120, the test article was delivered using three subcutaneous passes, 130, at approximately 300, 450, and 60° creating a fan pattern. An approximately equal volume, about 1.6 to about 1.7 mL, was delivered on each pass. A total volume of approximately 10 mL was delivered. Tested articles comprised particles of the invention suspended in a vehicle against vehicle only.

Subjects were divided into six cohorts. Each cohort contained two sub-groups. For each sub-group, N=3. The table below describes each cohort and sub-group.

| | | | Total bupivacaine, |
|---|---|---|---|
| Group | Leg 1 | Leg 2 | mg |
| 1a | 865B | Vehicle | 150 |
| 1b | 865A | Vehicle | 150 |
| 2a | 865B | Vehicle | 225 |
| 2b | 865A | Vehicle | 225 |
| 3a | 865B | Vehicle | 300 |
| 3b | 865A | Vehicle | 300 |
| 4a | 865B | Vehicle | 450 |
| 4b | 865A | Vehicle | 450 |
| 5b | 865A | Vehicle | 600 |

Briefly, each cohort was divided into two sub-groups of three subjects. For example, cohorts 1-5, one sub-group will receive particles of the invention made as described in Example 1B, suspended in a vehicle described herein having a viscosity greater than 50 cps in one leg and vehicle only in the other leg. The other sub-group will receive particles of the invention made as described in Example 1A in one leg and vehicle only in the other leg.

Clinical Outcomes:

Cohort 1:

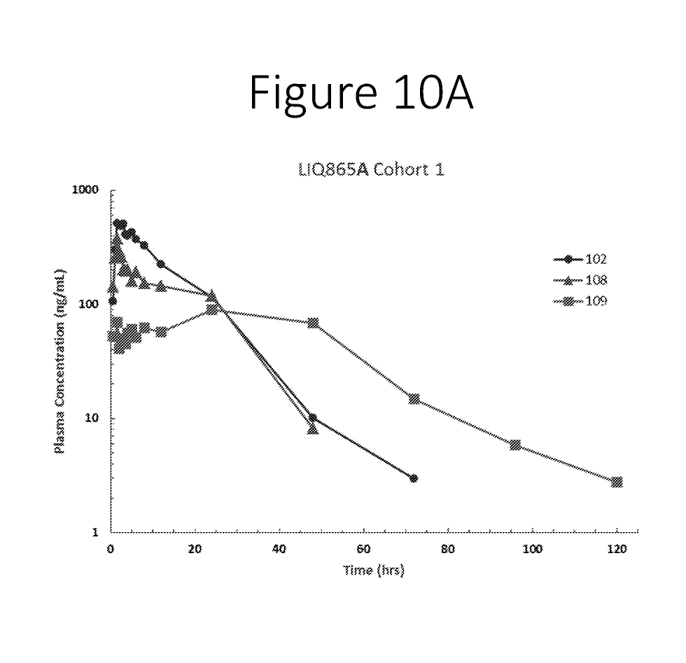

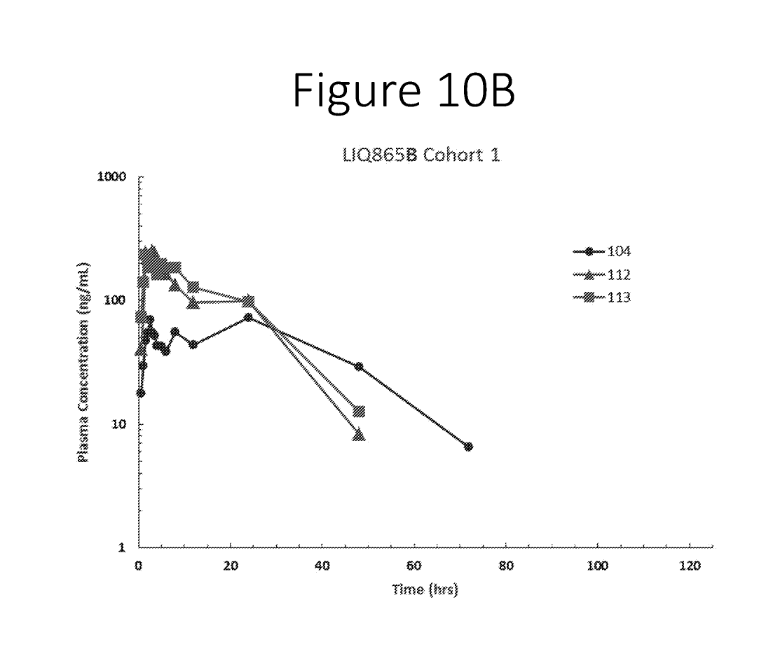

Generally, release from the particle resulted in evidence of onset at about the 1 h time-point and qualitative responses appeared across the tests. Plasma concentration over time is shown in the plots, per subject, in FIGS. 10A and 10B. FIG. 10A shows the plasma concentrations for subjects administered LIQ865A. FIG. 10B shows the plasma concentrations for subjects administered LIQ865B.

Cohort 2:

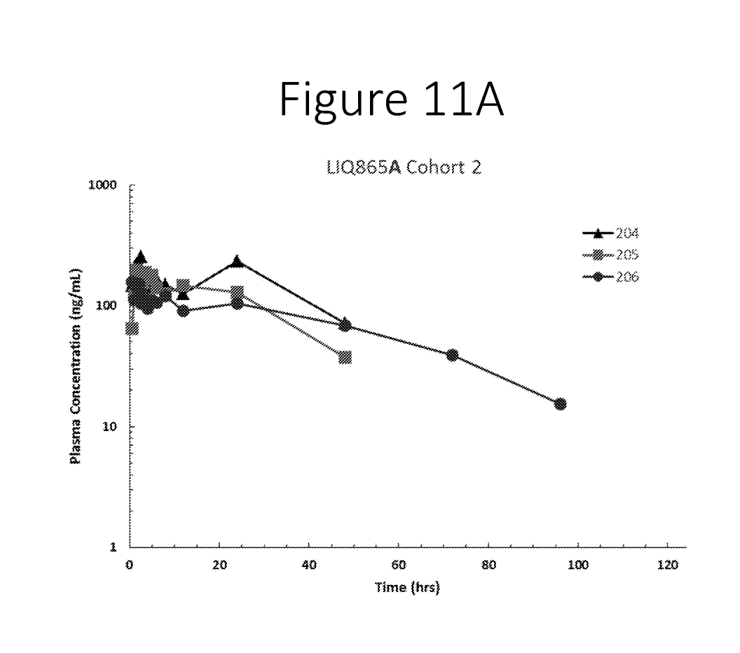

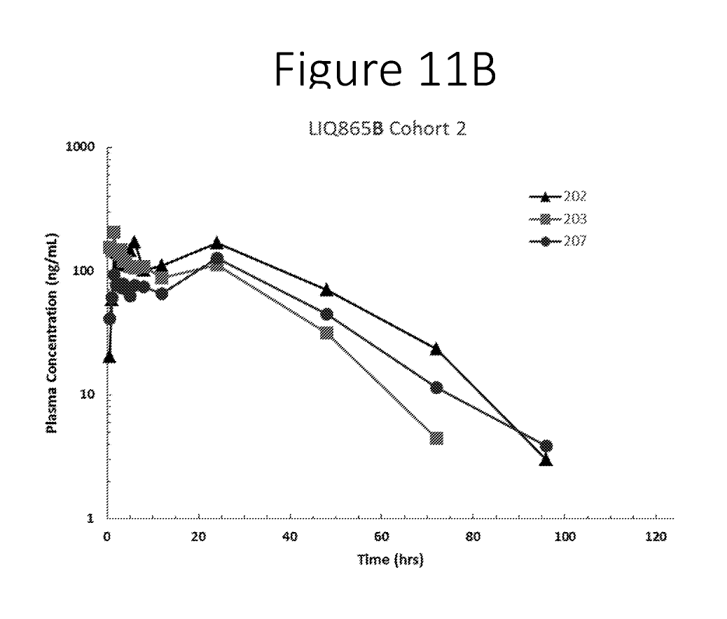

Generally, release from the particle resulted in PD assessments demonstrating onset of action (reduced sensitivity) occurring at or within 1 hour in all subjects with stronger effects in higher dosing cohort 2 compared to cohort 1. Duration of effect appeared to be up to or exceed 3 days for most sensitivity testing with a longer duration observed in cohort 2 compared to cohort 1. Plasma concentration over time is shown in the plots, per subject, in FIGS. 11A and 11B. FIG. 11A shows the plasma concentrations for subjects administered LIQ865A. FIG. 11B shows the plasma concentrations for subjects administered LIQ865B.

Cohort 3:

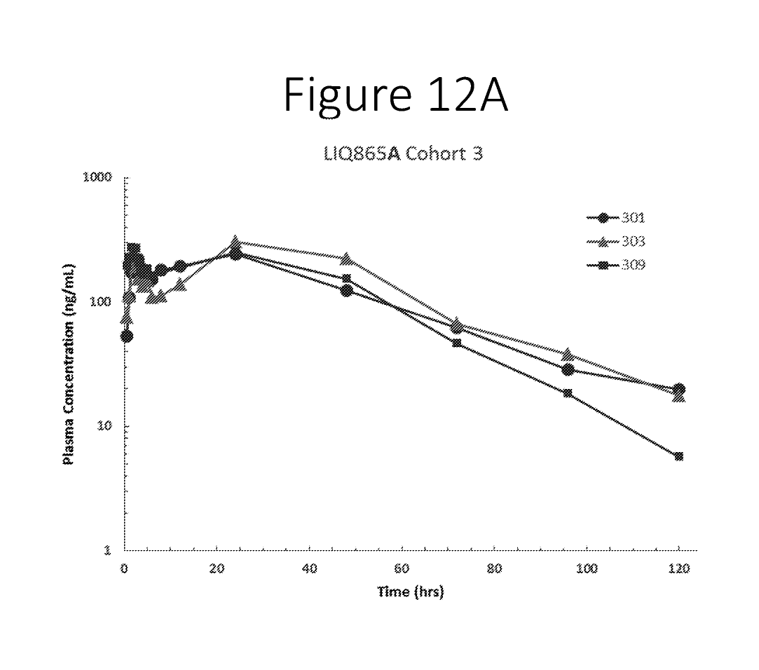

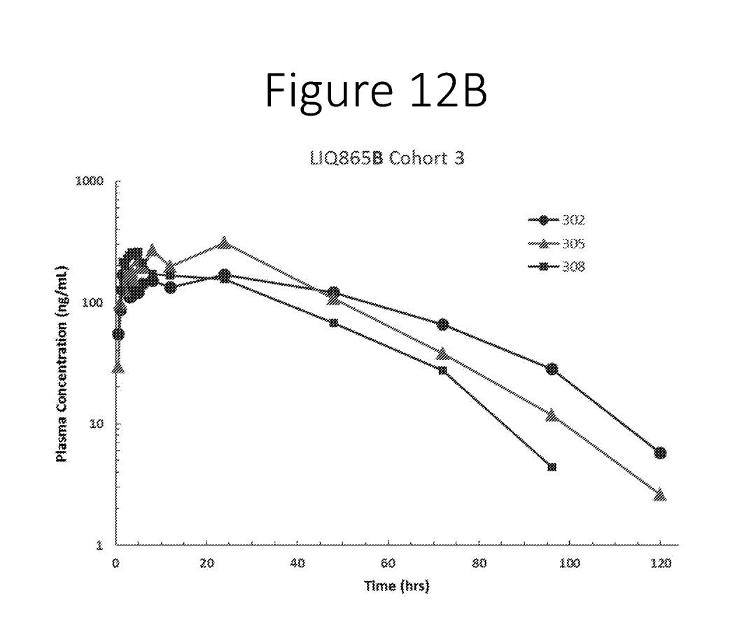

Generally, release from the particle resulted in PD assessments demonstrating onset of action (reduced sensitivity) occurring at or within 1 hour in all subjects with stronger effects in higher dosing cohort 3 compared to cohorts 1 and 2. Duration of effect appeared to be up to or exceed 3 days for most sensitivity testing with a longer duration observed in cohort 3 compared to cohorts 1 and 2. Plasma concentration over time is shown in the plots shown in FIGS. 12A and 12B. FIG. 12A shows the plasma concentrations for subjects administered LIQ865A. FIG. 12B shows the plasma concentrations for subjects administered LIQ865B.

Cohort 4:

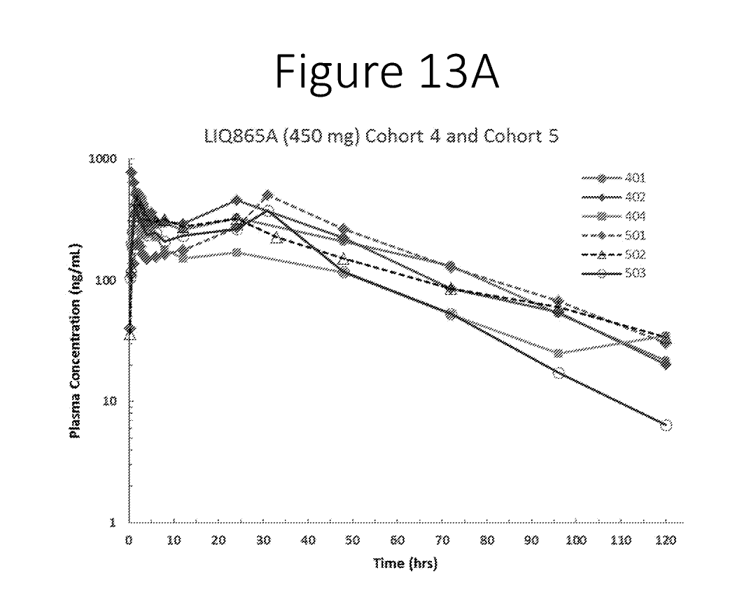

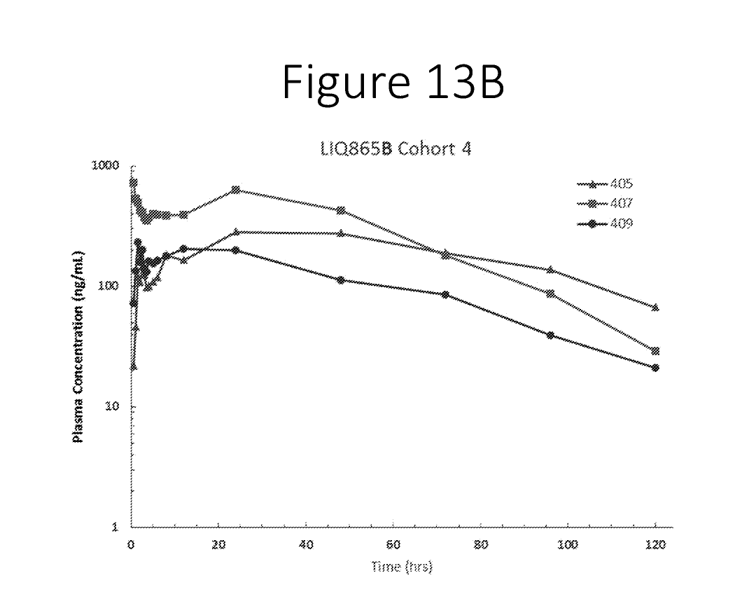

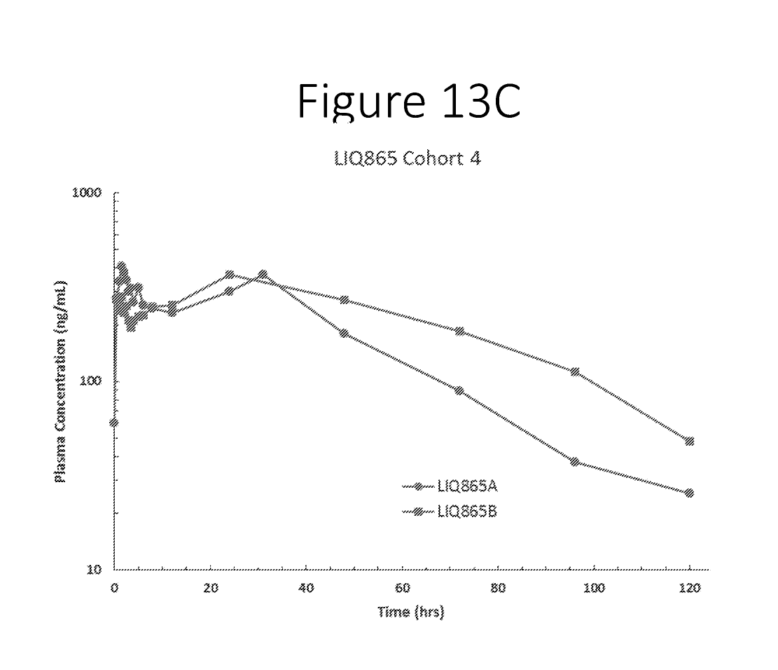

Generally, release from the particle resulted in PD assessments demonstrating onset of action (reduced sensitivity) occurring at or within 1 hour in all subjects with stronger effects in higher dosing cohort 4 compared to cohorts 1, 2 and 3. Duration of effect appeared to be up to or exceed 3 days for most sensitivity testing with a longer duration observed in cohort 4 compared to cohorts 1, 2 and 3. Plasma concentration over time is shown in the plots shown in FIGS. 13A and 13B. FIG. 13A shows the plasma concentrations for subjects administered LIQ865A in both Cohort 4 and Cohort 5 (see the description below for Cohort 5). FIG. 13B shows the plasma concentrations for subjects administered LIQ865B. FIG. 13C summarizes the data for the two drug particle designs. As in FIG. 13A, the additional 450 mg subjects from Cohort 5 are included in the consolidated graph.

Cohort 5

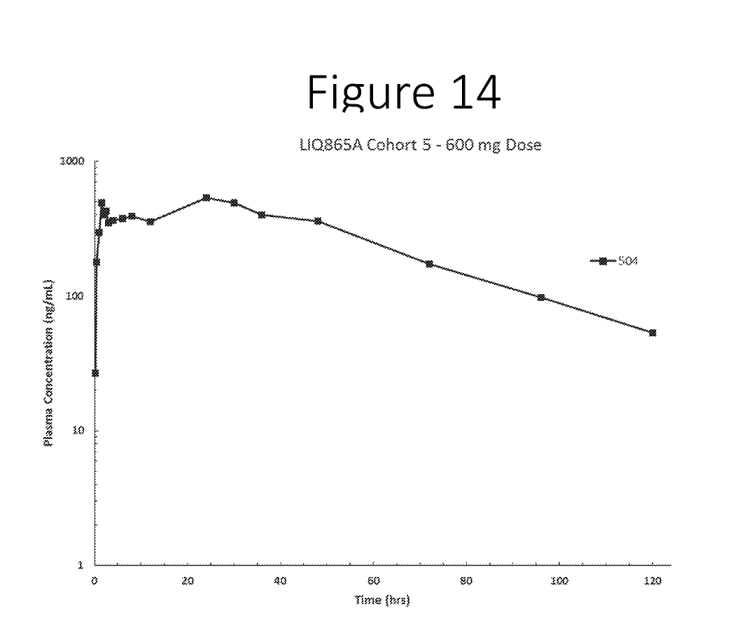

In Cohort 5, one subject was dosed with 600 mg and three additional subjects were dosed with 450 mg. Generally, release from the particle resulted in PD assessments demonstrating onset of action (reduced sensitivity) similar to that observed in Cohort 4 and providing moderate blunting to 3 to 5 days. FIG. 14 presents a log-linear plot including data for the one 600 mg subject over 120 hours.

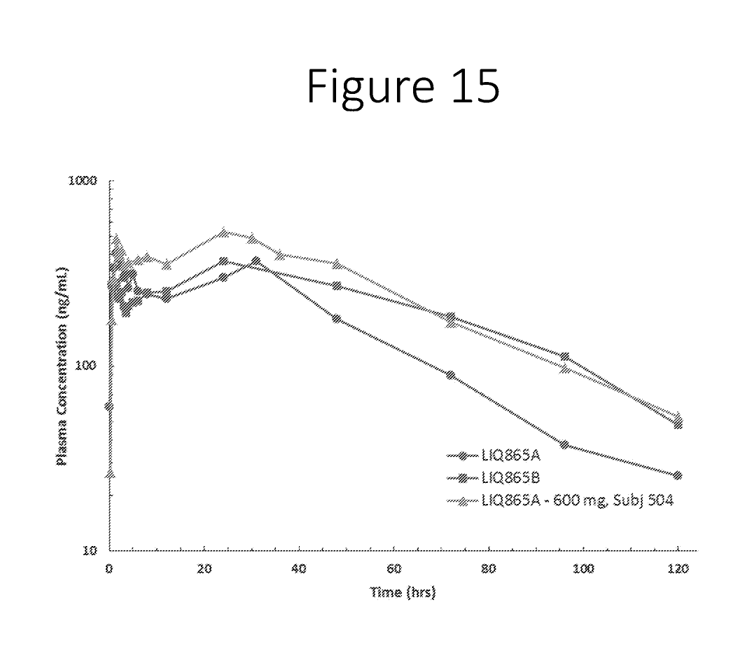

FIG. 15 presents a log-linear plot including data for all subjects dosed at 450 mg (in Cohorts 4 and 5) and the subject dosed at 600 mg.

Pharmacodynamic Summary

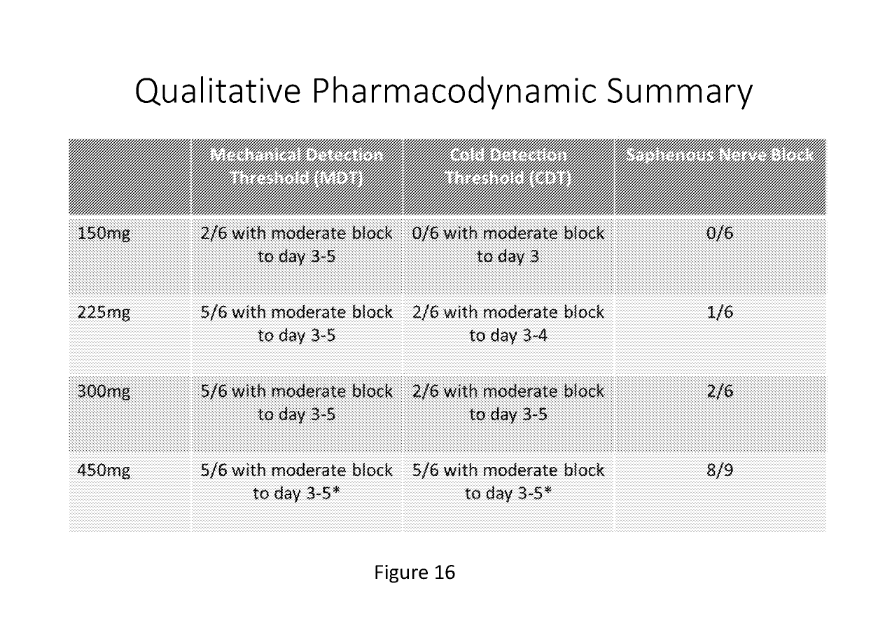

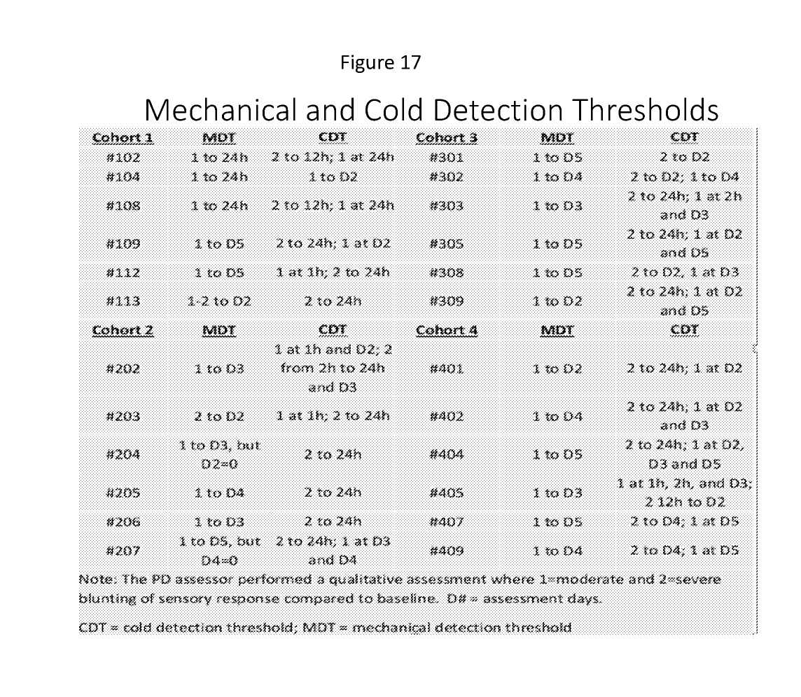

Pharmacodynamic data is presented in FIGS. 16 and 17. FIG. 16 presents a qualitative summary including the Mechanical Detection Threshold (MDT) and Cold Detection Threshold (CDT) for the 150 mg, 225 mg, 300 mg, and 450 mg doses. In the figure, the asterisks indicate that the pharmacodynamic analysis does not include the three additional 450 mg subjects in Cohort 5. FIG. 17 details the MDT and CDT data for the individual subjects in Cohorts 1-4. As in FIG. 16, the pharmacodynamics analysis of FIG. 17 does not show the three additional 450 mg subjects in Cohort 5.

Formulation 865A and Formulation 865B Data:

Particle formulation 865A comprises the particles fabricated with PLGA matrix material and the amino amide anesthetic API. Particle formulation 865B comprises the particles fabricated without a polymeric matrix material or other excipient and solely the amino amide anesthetic API.

In some embodiments, formulation A may cause a pH shift with degradation of the PLGA, thereby encouraging ionization of the bupivacaine and ultimately leading to greater solubility of the active agent.

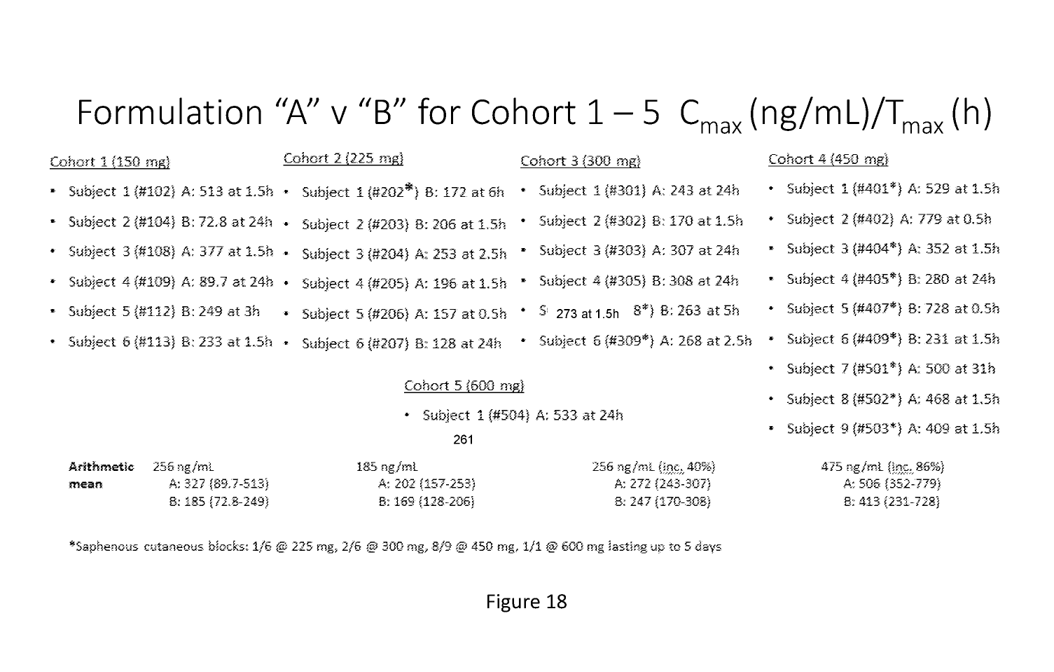

FIG. 18 shows the particles of the present invention including PLGA polymer matrix resulted in a higher ng/mL blood concentration (Cₘₐₓ) than particle formulations without polymer matrix material. In some embodiments, 150 mg dose of bupivacaine in particles with PLGA resulted in arithmetic mean concentration of 327 ng/mL compared to the same dose of bupivacaine from particles without polymer matrix material having arithmetic mean concentration of 185 ng/mL. In some embodiments, 225 mg dose of bupivacaine in particles with PLGA resulted in arithmetic mean concentration of 202 ng/mL compared to the same dose of bupivacaine from particles without polymer matrix material having arithmetic mean concentration of 169 ng/mL. In some embodiments, 300 mg dose of bupivacaine in particles with PLGA resulted in arithmetic mean concentration of 272 ng/mL compared to the same dose of bupivacaine from particles without polymer matrix material having arithmetic mean concentration of 247 ng/mL. In some embodiments, 450 mg dose of bupivacaine in particles with PLGA resulted in arithmetic mean concentration of 506 ng/mL compared to the same dose of bupivacaine from particles without polymer matrix material having arithmetic mean concentration of 413 ng/mL. As shown in FIG. 18, in some embodiments, additional subjects in the 450 mg dose range showed confirmatory results to the earlier subjects at the same dose and a single subject 600 mg dose of bupivacaine particles with PLGA resulted in a Cₘₐₓ of 533 ng/mL at 24 hours.

Nerve Block

In some embodiments, the particles of the present invention are useful for nerve block applications lasting for up to 5 days. Given that some of the superficial cutaneous sensory branches of the saphenous nerve (SN) distal to the knee pass deep to the injection site, it is perhaps not surprising that several subjects developed distal medial cutaneous sensory nerve the leg branch blocks in addition to blunted sensory responses inside the test area. SN-blocks were seen in 1/6 in Cohort 2 (225 mg), 2/6 in Cohort 3 (300 mg), 5/6 in Cohort 4 (450 mg), 3/3 in Cohort 5 (450 mg), and 1/1 in Cohort 5 (600 mg). These conduction blocks support the profile of 3-5 days duration of nerve block.

1-10. (canceled)

11. A method of inducing extended analgesia, comprising:
administering to a site in need a composition comprising a plurality of particles, each particle of the plurality comprising 40-60 wt % amino amide anesthetic or a pharmaceutically acceptable salt, hydrate, or solvate thereof and 60-40 wt % PLGA polymer comprising 48:52 to 52:48 molar ratio D,L lactide:glycolide and an inherent viscosity of about 0.16 to 0.24 dL/g at 0.1% w/v in chloroform at 25° C., wherein each particle comprises a non-spherical shape less than 100 μm in a broadest dimension; and
whereby the particles provide 3 or more days of analgesia to the site in need.

12. The method of claim 11, further comprising before administering, suspending the particles in a vehicle comprising a viscosity modifier, a surfactant, a buffer, and, a tonicity modifier, wherein the vehicle comprises a viscosity less than about 50 cps.

13. The method of claim 12, further comprising before suspending the particle in a vehicle, formulating the vehicle with a viscosity less than about 50 cps.

14. The method of claim 11, wherein administering comprises infiltration, injection or topical administration.

15. The method of claim 11, wherein each particle of the plurality has a volume of about 13,500 cubic micrometers and a surface area of about 3500 square micrometers.

16. The method of claim 11, wherein the amino amide anesthetic is crystalline and comprises 50-70% crystalline form I and 30-50% crystalline form II.

17. The method of claim 11, wherein the amino amide anesthetic comprises bupivacaine free base or pharmaceutically acceptable salts, hydrates, and solvates thereof.

18. The method of claim 12, wherein the viscosity modifier comprises sodium hyaluronate having an inherent viscosity of 1.6 to 2.2 m³/kg and comprises about 0.5 to about 1.0 wt % of the vehicle, and wherein the surfactant comprises polysorbate 80, polysorbate 20, docusate sodium or sodium deoxycholate and the vehicle optionally comprises a co-solvent comprising ethanol, benzyl alcohol or glycerin comprising from about 0.001 to 1.0 wt % of the vehicle.

19. A formulation for administration to induce analgesia, comprising:
a plurality of particles suspended in a vehicle comprising about 0.1 to 0.3 wt % viscosity modifier, about 4.0 wt % tonicity modifier, about 0.1 wt % surfactant, about 0.6 wt % buffer, a pH of about 7.7 to 8.3, and viscosity of about 30 to 50 cps;
wherein each particle of the plurality comprises 40-60 wt % amino amide anesthetic or a pharmaceutically acceptable salt, hydrate, or solvate thereof and 60-40 wt % PLGA polymer comprising 48:52 to 52:48 molar ratio D,L lactide:glycolide and an inherent viscosity of about 0.16 to 0.24 dL/g at 0.1% w/v in chloroform at 25° C.;
wherein each particle comprises a non-spherical shape less than 100 μm in a broadest dimension and having a volume of about 13,500 cubic micrometers; and
wherein the amino amide anesthetic is crystalline and comprises 50-70% crystalline form I and 30-50% crystalline form II.

20. The formulation of claim 19, wherein the amino amide anesthetic comprises bupivacaine free base or a pharmaceutically acceptable salt, hydrate, or solvate thereof.

21. The formulation of claim 19, wherein each particle comprises a surface area of about 3500 square micrometers.

22. A method of forming an anesthetic particle, comprising:
depositing a solution comprising 40-60 wt % amino amide anesthetic and 60-40 wt % PLGA onto a polymer mold comprising cavities having a volume of about 13500 cubic micrometers;
positioning the solution into the cavities of the mold; and
drying the solution while in the mold cavities to form crystalline amino amide anesthetic PLGA anesthetic particles, wherein the crystalline amino amide anesthetic comprises between 50-70% crystalline form I and 30-50% crystalline form 11.34.

23. A composition comprising:
a plurality of particles, each particle of the plurality comprising 40-60 wt % amino amide anesthetic or a pharmaceutically acceptable salt, hydrate, or solvate thereof and 60-40 wt % PLGA polymer comprising 48:52 to 52:48 molar ratio D,L lactide:glycolide and an inherent viscosity of about 0.16 to 0.24 dL/g at 0.1% w/v in chloroform at 25° C.;
wherein each particle comprises a non-spherical shape less than 100 μm in a broadest dimension, and having a volume of about 13,500 cubic micrometers; and
wherein the amino amide anesthetic is crystalline and comprises 50-70% crystalline form I and 30-50% crystalline form II.

24. The composition of claim 23, wherein the amino amide anesthetic is selected from the group consisting of dibucaine, lidocaine, mepivacaine, prilocaine, bupivacaine, levobupivacaine, ropivacaine, articaine, etidocaine, and pharmaceutically acceptable salts, hydrates, and solvates thereof.

25. The composition of claim 23, wherein the amino amide anesthetic comprises bupivacaine free base or pharmaceutically acceptable salts, hydrates, and solvates thereof.

26. The composition of claim 23, wherein the particle comprises a surface area of about 3500 square micrometers.

27. The composition of claim 23, further comprising an aqueous vehicle comprising a viscosity modifier, a surfactant, a buffer, and, a tonicity modifier, wherein the vehicle comprises a viscosity less than about 50 cps.

28. The composition of claim 27, wherein the viscosity modifier comprises hyaluronic acid or a pharmaceutically acceptable salt thereof.

29. The composition of claim 27, wherein the viscosity modifier comprises sodium hyaluronate having an inherent viscosity of 1.6 to 2.2 m³/kg.

30. The composition of claim 27, wherein the viscosity modifier comprises sodium hyaluronate comprising about 0.5 to about 1.0 wt % of the vehicle.

31. The composition of claim 27, wherein the surfactant comprises polysorbate 80 or polysorbate 20 comprising from about 0.001 to 1.0 wt % of the vehicle.

32. The composition of claim 27, wherein the vehicle further comprises a surfactant selected from docusate sodium or sodium deoxycholate and optionally a co-solvent comprising ethanol, benzyl alcohol or glycerin.
